(19) United States
(12) Patent Application Publication
(10) Pub. No.: US 2016/0185821 A1
(43) Pub. Date: Jun. 30, 2016

(54) Z-SELECTIVE OLEFIN METATHESIS OF PEPTIDES

(71) Applicant: CALIFORNIA INSTITUTE OF TECHNOLOGY, Pasadena, CA (US)

(72) Inventors: Shane L. MANGOLD, Midland, MI (US); Robert H. GRUBBS, South Pasadena, CA (US)

(21) Appl. No.: 14/802,244

(22) Filed: Jul. 17, 2015

Related U.S. Application Data
(60) Provisional application No. 62/026,426, filed on Jul. 18, 2014.

(51) Int. Cl. C07K 7/08; C07D 307/79; C07K 7/06; C07C 271/22

(52) U.S. Cl. CPC C07K 7/08; C07C 271/22; C07D 307/79; C07K 7/06

(57) ABSTRACT

The invention relates generally to the synthesis of modified amino acids and modified peptides in the presence of cyclometalated catalysts. The invention has utility in the fields of catalysis, organic synthesis, polymer chemistry, and industrial and fine chemicals chemistry.

### CROSS REFERENCE TO RELATED APPLICATIONS

The present application claims the benefit of U.S. Provisional Patent Application No. 62/026,426, filed Jul. 18, 2014, which is incorporated herein by reference in its entirety.

### STATEMENT OF FEDERAL SUPPORT

This invention was made with government support under GM031332 awarded by the National Institutes of Health. The government has certain rights in the invention.

### TECHNICAL FIELD

The invention relates generally to the synthesis of modified amino acids and modified peptides in the presence of cyclometalated catalysts. The invention has utility in the fields of catalysis, organic synthesis, polymer chemistry, and industrial and fine chemicals chemistry.

### BACKGROUND

Olefin metathesis is a highly versatile tool for the generation of carbon-carbon bonds and a variety of applications have evolved around its implementation (see Furstner, A. Angew. Chem. Int. Ed. 2000, 39, 3012; Mol, J. J. Mol. Catal. A-Chem. 2004, 213, 39; Samojlowicz, C.; Grela, K. ARKIVOC 2011, 4, 71; Schrock, R. R. Chem. Rev. 2002, 102, 145; Trnka, T. M.; Grubbs, R. H. Acc. Chem. Res. 2001, 34, 18). The broad utility of olefin metathesis is a consequence of the exceptional selectivity, activity, and functional group compatibility of select metathesis catalysts, highlighted by carbon-carbon bond formation on a variety of complex substrates including small molecules, (see Mangold, S. L.; Prost, L. R.; Kiessling, L. L. Chem. Sci. 2012, 3, 772; Donohoe, T. J.; Fishlock, L. P.; Procopiou, P. A. Chem-Eur. J. 2008, 14, 5716; Donohoe, T. J.; Orr, A. J.; Bingham, M. Angew. Chem. Int. Ed. 2006, 45, 2664; Schreiber, S. L. Science 2000, 287, 1964; Schuster, M.; Blechert, S. Angew. Chem. Int. Ed. 1997, 36, 2036; Tatton, M. R. S., I. Donohoe, T. J. Org. Lett. 2014, 16, 1920) natural products, (see Hoveyda, A. H.; Malcolmson, S. J.; Meek, S. J.; Zhugralin, A. R. Angew. Chem. Int. Ed. 2010, 49, 34; Nicolaou, K. C.; Bulger, P. G.; Sarlah, D. Angew. Chem. Int. Ed. 2005, 44, 4490) organic and inorganic materials, (see Kim, N. Y.; Jeon, N. L.; Choi, I. S.; Takami, S.; Harada, Y.; Finnie, K. R.; Girolami, G. S.; Nuzzo, R. G.; Whitesides, G. M.; Laibinis, P. E. Macromolecules 2000, 33, 2793; Leitgeb, A.; Wappel, J.; Slugovc, C. Polymer 2010, 51, 2927; Liu, X.; Basu, A. J. Organomet. Chem. 2006, 691, 5148; Sveinbjörnsson, B. R.; Weitekamp, R. A. M., G. M.; Xiaa, Y.; Atwater, H. A. G., R. H. Proc. Natl. Acad. Sci. U.S.A. 2012, 109, 14332; Weitekamp, R. A.; Atwater, H. A.; Grubbs, R. H. J. Am. Chem. Soc. 2013, 135, 16817) and even proteins (see Binder, J. B.; Raines, R. T. Curr. Opin. Chem. Biol. 2008, 12, 767; Lin, Y. A.; Chalker, J. M.; Davis, B. G. ChemBioChem 2009, 10, 959). The use of metathesis in biological applications is an emerging field of research, in part, due to advances in the genetic (see Song, W.; Wang, Y.; Qu, J.; Lin, Q. J. Am. Chem. Soc. 2008, 130, 9654; van Hest, J. C. M.; Kiick, K. L.; Tirrell, D. A. J. Am. Chem. Soc. 2000, 122, 1282; Zhang, Z.; Wang, L.; Brock, A.; Schultz, P. G. Angew. Chem. Int. Ed. 2002, 41, 2840) and chemical (see Bernardes, G. J. L.; Chalker, J. M.; Errey, J. C.; Davis, B. G. J. Am. Chem. Soc. 2008, 130, 5052; Lin, Y. A.; Boutureira, O.; Lercher, L.; Bhushan, B.; Paton, R. S.; Davis, B. G. J. Am. Chem. Soc. 2013, 135, 12156; Zhu, Y.; van der Donk, W. A. Org. Lett. 2001, 3, 1189) incorporation of alkene-containing amino acids into peptides and proteins. This has enabled the installation of a variety of carbon-carbon bonds with high fidelity for applications in peptide stapling, (see Miller, S. J.; Blackwell, H. E.; Grubbs, R. H. J. Am. Chem. Soc. 1996, 118, 9606; Blackwell, H. B.; Grubbs, R. H. Angew. Chem. Int. Ed. 1998, 37, 3281; Blackwell, H. B.; Sadowsky, J. D.; Howard, R. J.; Sampson, N. S.; Chao, J. A.; Steinmetz, W. E.; O'Leary, D. J.; Grubbs, R. H. J. Org. Chem. 2001, 66, 5291; Brown, C. J.; Quah, S. T.; Jong, J.; Goh, A. M.; Chiam, P. C.; Khoo, K. H.; Choong, M. L.; Lee, M. A.; Yurlova, L.; Zolghadr, K.; Joseph, T. L.; Verma, C. S.; Lane, D. P. ACS Chem. Biol. 2013, 8, 506; Gionnet-Estieu, K.; Guichard, G. Exp. Opin. Drug Discov. 2011, 6, 937) as surrogates of hydrogen-bonding, (see Verdine, G. L.; Hilinski, G. J. Methods Enzymol. 2012, 503, 3; Verdine, G. L.; Walensky, L. D. Clin. Cancer Res. 2007, 13, 7264; Walensky, L. D.; Kung, A. L.; Escher, I.; Malia, T. J.; Barbuto, S.; Wright, R. D.; Wagner, G.; Verdine, G. L.; Korsmeyer, S. J. Science 2004, 305, 1466) and as methods for modifications of peptides and proteins used to mimic physiologically relevant post-translational modifications (see Lin, Y. A.; Chalker, J. M.; Davis, B. G. ChemBioChem 2009, 10, 959; Henchey, L. K.; Jochim, A. L.; Arora, P. S. Curr. Opin. Chem. Biol. 2008, 12, 692). The application of metathesis for stabilizing peptide secondary structure and in selective protein modification has implications for imparting greater metabolic stability, cellular permeability, and higher binding affinity toward biological targets (see Miller, S. J.; Blackwell, H. E.; Grubbs, R. H. J. Am. Chem. Soc. 1996, 118, 9606; Liu, J.; Wang, D.; Zheng, Q.; Lu, M.; Arora, P. S. J. Am. Chem. Soc. 2008, 130, 4334; Patgiri, A.; Jochim, A. L.; Arora, P. S. Acc. Chem. Res. 2008, 41, 1289; Lin, Y. A.; Chalker, J. M.; Davis, B. G. J. Am. Chem. Soc. 2010, 132, 16805).

Indeed, this strategy has led to the development of ‘stapled’ peptides used as inhibitors of HIV fusion (see Bernal, F.; Wade, M.; Godes, M.; Davis, T. N.; Whitehead, D. G.; Kung, A. L.; Wahl, G. M.; Walensky, L. D. Cancer cell 2010, 18, 411) and assembly (see Phillips, C.; Roberts, L. R.; Schade, M.; Bazin, R.; Bent, A.; Davies, N. L.; Moore, R.; Pannifer, A. D.; Pickford, A. R.; Prior, S. H.; Read, C. M.; Scott, A.; Brown, D. G.; Xu, B.; Irving, S. L. J. Am. Chem. Soc. 2011, 133, 9696; Schafmeister, C. E.; Po, J.; Verdine, G. L. J. Am. Chem. Soc. 2000, 122, 5891; Bird, G. H.; Madani, N.; Perry, A. F.; Princiotto, A. M.; Supko, J. G.; He, X.; Gavathiotis, E.; Sodroski, J. G.; Walensky, L. D. Proc. Natl. Acad. Sci. U.S.A 2010, 107, 14093; Bhattacharya, S.; Zhang, H.; Debnath, A. K.; Cowburn, D. J. Biol. Chem. 2008, 283, 16274) as modulators of signaling pathways involved in cancer, (Long, Y. Q.; Huang, S. X.; Zawahir, Z.; Xu, Z. L.; Li, H.; Sanchez, T. W.; Zhi, Y.; DeHouwer, S.; Christ, F.; Debyser, Z.; Neamati, N. J. Med. Chem. 2013, 56, 5601; Zhang, H.; Curreli, F.; Waheed, A. A.; Mercredi, D. Y.; Mehta, M.; Bhargava, P.; Scacalossi, D.; Tong, X.; Lee, S.; Cooper, A.; Summers, M. F.; Freed, E. O.; Debnath, A. K. Retrovirology 2013, 10, 136; Zhang, H.; Zhao, Q.; Bhattacharya, S.; Waheed, A. A.; Tong, X.; Hong, A.; Heck, S.; Curreli, F.; Goger, M.; Cowburn, D.; Freed, E. O.; Debnath, A. K. J. Mol. Biol. 2008, 378, 565; Chang, Y. S.; Gravesb, B.; Guerlavaisa, V.; Tovarb, C.; Packmanb, K.; Tob, K.-H.; Olsona, K. A.; Kesavana, K.; Gangurdea, P.; Mukherjeea, A.; Bakera, T.; Darlaka, K.; Elkina, C.; Filipovich, Z.; Qureshib, F. Z.; Caia, H.; Berry, P.; Feyfanta, E.; Shia, X. E.; Horsticka, J.; Annisa, D. A.; Manninga, A. M.; Fotouhib, N.; Nasha, H.; Vassilev, L. T.; Sawyer, T. K. Proc. Natl. Acad. Sci. U.S.A 2013, 110, e3445) and in selective activation of enzymes involved in diabetes (see Takada, K.; Zhu, D.; Bird, G. H.; Sukhdeo, K.; Zhao, J. J.; Mani, M.; Lemieux, M.; Carrasco, D. E.; Ryan, J.; Horst, D.; Fulciniti, M.; Munshi, N. C.; Xu, W.; Kung, A. L.; Shivdasani, R. A.; Walensky, L. D.; Carrasco, D. R. Sci. Transl. Med. 2012, 4, 148ra117; Hao, Y.; Wang, C.; Cao, B.; Hirsch, B. M.; Song, J.; Markowitz, S. D.; Ewing, R. M.; Sedwick, D.; Liu, L.; Zheng, W.; Wang, Z. Cancer cell 2013, 23, 583).

Despite the tremendous success of metathesis in peptide and peptidomimetic research, the ability to control olefin geometry in the product has been met with limited success, (see Grossmann, T. N.; Yeh, J. T. H.; Bowman, B. R.; Chu, Q.; Moellering, R. E.; Verdine, G. L. Proc. Natl. Acad. Sci. U.S.A 2012, 109, 17942, Danial, N. N.; Walensky, L. D.; Zhang, C.-Y.; Choi, C. S.; Fisher, J. K.; Molina, A. J. A.; Datta, S. R.; Pitter, K. L.; Bird, G. H.; Wikstrom, J. D.; Deeney, J. T.; Robertson, K.; Morash, J.; Kulkarni, A.; Neschen, S.; Kim, S.; Greenberg, M. E.; Corkey, B. E.; Shirihai, O. S.; Shulman, G. I.; Lowell, B. B.; Korsmeyer, S. J. Nat. Med. 2008, 14, 144; Szlyk, B.; Braun, C. R.; Ljubicic, S.; Patton, E.; Bird, G. H.; Osundiji, M. A.; Matschinsky, F. M.; Walensky, L. D.; Danial, N. N. Nat. Struct. Mol. Biol. 2014, 21, 36). Most metathesis catalysts exhibit minimal kinetic selectivity, and thus, the product distribution reflects the thermodynamic stability of each olefin isomer, (see Lee, C. W.; Grubbs, R. H. Org. Lett. 2000, 2, 2145). In many cases, a mixture of E and Z isomers is formed, that is often inseparable. This imposes challenges for examining the influence of alkene geometry on the stability and activity of diverse compounds. In pursuit of catalysts with greater control over olefin products, a series of cyclometalated ruthenium catalysts that could achieve high conversions with Z-selectivity (Scheme 1) were discovered (see Wang, Y.; Jimenez, M.; Hansen, A.; Raiber, E. A.; Schreiber, S. L.; Young, D. W. J. Am. Chem. Soc. 2011, 133, 9196; Prunet, J. Angew. Chem. Int. Ed. 2003, 42, 2826; Grubbs, R. H. In Hanbook of Metathesis; Wiley-VCH: Weinheim, Germany, 2003; Vol. 1; Keitz, B. K.; Endo, K.; Patel, P. R.; Herbert, M. B.; Grubbs, R. H. J. Am. Chem. Soc. 2012, 134, 693; Quigley, B. L.; Grubbs, R. H. Chem. Sci. 2014, 5, 501; Rosebrugh, L. E.; Herbert, M. B.; Marx, V. M.; Keitz, B. K.; Grubbs, R. H. J. Am. Chem. Soc. 2013, 135, 1276).

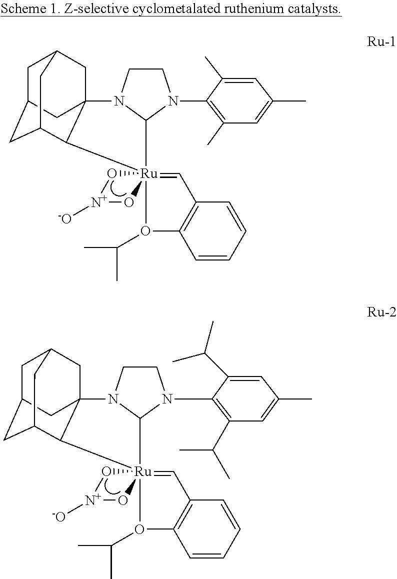

The origin of Z-selectivity for cyclometalated ruthenium catalysts involves approach of the olefin from a side-bound position (i.e., cis to the N-heterocyclic carbene (“NHC”) ligand and trans to the chelating adamantyl) which is favored through a combination of steric and electronic effects imposed by the NHC ligand, (see Endo, K.; Grubbs, R. H. J. Am. Chem. Soc. 2011, 133, 8525). While catalysts Ru-1 and Ru-2 demonstrate excellent selectivity in olefin metathesis, their activity on complex substrates, including peptides, remained unexplored. To this end, a comprehensive evaluation of Z-selective metathesis of peptides using newly developed cyclometalated ruthenium catalysts was initiated. Through the combined efforts of homodimerization, cross metathesis and ring-closing metathesis, guidelines for assessing the influence of amino acids and peptides on catalyst activity and selectivity were developed. These principles were applied for carrying out Z-selective metathesis on challenging substrates including peptides that comprise parallel β-sheets and on stapling of α-helical peptides.

The emergence of chemical strategies for accessing macrocyclic motifs has fostered a renewed interest in their development and macrocycles now fulfill roles in diverse applications from natural products, (see Nicolaou, K. C.; Bulger, P. G.; Sarlah, D. Angew. Chem. Int. Ed. 2005, 44, 4490; Yu, X.; Sun, D. Molecules 2013, 18, 6230) and therapeutics (see Driggers, E. M.; Hale, S. P.; Lee, J.; Terrett, N. K. Nat. Rev. Drug Discov. 2008, 7, 608; Mallinson, J.; Collins, I. Future Med. Chem. 2012, 4, 1409) to platforms in supramolecular chemistry, (see Diederich, F.; Stang, P. J.; Tykwinski, R. R. Modern Supramolecular Chemistry: Strategies for Macrocycle Synthesis; Wiley-VCH: Weinheim, Germany, 2008). Contemporary strategies for macrocycle formation often rely on the use of macrolactonization, (see Parenty, A.; Moreau, X.; Campagne, J. M. Chem. Rev. 2006, 106, 911; Swamy, K. C.; Kumar, N. N.; Balaraman, E.; Kumar, K. V. Chem. Rev. 2009, 109, 2551; Wu, X.-F.; Neumann, H.; Beller, M. Chem. Rev. 2013, 113, 1) macrolactamization, (see Wen, S.; Packham, G.; Ganesan, A. J. Org. Chem. 2008, 73, 9353; White, C. J.; Yudin, A. K. Nat. Chem. 2011, 3, 509; Song, Z. J.; Tellers, D. M.; Journet, M.; Kuethe, J. T.; Lieberman, D.; Humphrey, G.; Zhang, F.; Peng, Z.; Waters, M. S.; Zewge, D.; Nolting, A.; Zhao, D.; Reamer, R. A.; Dormer, P. G.; Belyk, K. M.; Davies, I. W.; Devine, P. N.; Tschaen, D. M. J. Org. Chem. 2011, 76, 7804), “click” cyclization, (see Turner, R. A.; Oliver, A. G.; Lokey, R. S. Org. Lett. 2007, 9, 5011; Chouhan, G.; James, K. Org. Lett. 2011, 13, 2754; Pasini, D. Molecules 2013, 18, 9512) or transition-metal catalyzed reactions including olefin metathesis, (see Gradillas, A.; Perez-Castells, J. Angew. Chem. Int. Ed. 2006, 45, 6086; Hoveyda, A. H.; Zhugralin, A. R. Nature 2007, 450, 243) and intramolecular cross coupling, (see Nicolaou, K. C.; Bulger, P. G.; Sarlah, D. Angew. Chem. Int. Ed. 2005, 44, 4490; Chemler, S. R.; Trauner, D.; Danishefsky, S. J. Angew. Chem. Int. Ed. 2001, 40, 4544; Evano, G.; Blanchard, N.; Toumi, M. Chem. Rev. 2008, 108, 3054). Among these strategies, ring-closing metathesis (RCM) has assumed a prominent role in macrocycle formation, in part, as a consequence of the selectivity and functional group compatibility of select olefin metathesis catalysts, (see Maier, M. E. Angew. Chem. Int. Ed. 2000, 39, 2073; Vougioukalakis, G. C.; Grubbs, R. H. Chem. Rev. 2010, 110, 1746).

Such chemoselectivity has offered new strategies for retrosynthetic disconnections in complex molecule synthesis and many active pharmaceuticals have been developed around the use of RCM, (see Wei, X.; Shu, C.; Haddad, N.; Zeng, X.; Patel, N. D.; Tan, Z.; Liu, J.; Lee, H.; Shen, S.; Campbell, S.; Varsolona, R. J.; Busacca, C. A.; Hossain, A.; Yee, N. K.; Senanayake, C. H. Org. Lett. 2013, 15, 1016; Erb, W.; Zhu, J. Nat. Prod. Rep. 2013, 30, 161). One promising application of RCM involves macrocyclization on peptides, often conferring beneficial properties to these compounds including enhanced activity, (see Walensky, L. D.; Kung, A. L.; Escher, I.; Malia, T. J.; Barbuto, S.; Wright, R. D.; Wagner, G.; Verdine, G. L.; Korsmeyer, S. J. Science 2004, 305, 1466; Moellering, R. E.; Cornejo, M.; Davis, T. N.; Del Bianco, C.; Aster, J. C.; Blacklow, S. C.; Kung, A. L.; Gilliland, D. G.; Verdine, G. L.; Bradner, J. E. Nature 2009, 462, 182; Walensky, L. D.; Bird, G. H. J. Med. Chem. 2014, 57, 6275) and improved proteolytic stability, (see Verdine, G. L.; Hilinski, G. J. Methods Enzymol. 2012, 503, 3; Bird, G. H.; Gavathiotis, E.; LaBelle, J. L.; Katz, S. G.; Walensky, L. D. ACS Chem. Biol. 2014, 9, 831). While RCM has found utility across many disciplines, an outstanding challenge in this transformation has been the ability to control olefin geometry in the product. Although indirect methods have been developed, including alkyne metathesis followed by partial reduction, (see Furstner, A.; Guth, O.; Rumbo, A.; Seidel, G. J. Am. Chem. Soc. 1999, 121, 11108; Furstner, A.; Davies, P. W. Chem. Commun. 2005, 2307; Zhang, W.; Moore, J. S. Adv. Synth. Catal. 2007, 349, 93; Furstner, A. Angew. Chem. Int. Ed. 2013, 52, 2794) or substrate-controlled RCM of vinylsiloxanes followed by desilylation, (see Wang, Y.; Jimenez, M.; Hansen, A. S.; Raiber, E. A.; Schreiber, S. L.; Young, D. W. J. Am. Chem. Soc. 2011, 133, 9196; Gallenkamp, D.; Furstner, A. J. Am. Chem. Soc. 2011, 133, 9232) the scope of these transformations is limited. A more streamlined route was envisioned that could be devised by modulating the equilibrium of olefin metathesis with control over both RCM and the reverse, ring-opening metathesis (ROM) using ethylene and olefin selective metathesis catalysts (Scheme 2).

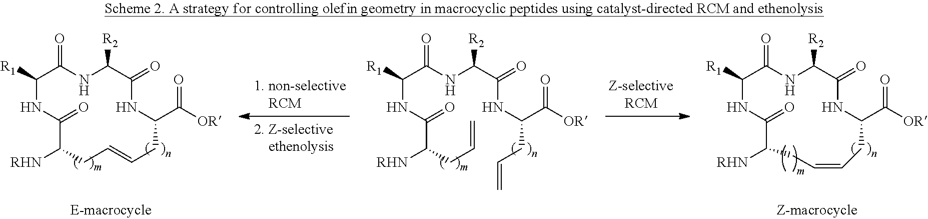

The use of Z-selective cyclometalated ruthenium catalysts for the derivatization of commodity chemical feedstocks using catalyst-controlled ethenolysis was recently explored (see Marx, V. M.; Herbert, M. B.; Keitz, B. K.; Grubbs, R. H. J. Am. Chem. Soc. 2013, 135, 94; Marx, V. M.; Sullivan, A. H.; Melaimi, M.; Virgil, S. C.; Keitz, B. K.; Weinberger, D. S.; Bertrand, G.; Grubbs, R. H. Angew. Chem. Int. Ed. 2015, 54, 1919). These studies led us to consider whether Z-selective ethenolysis could serve as a practical tool for the purification of E-olefins from stereoisomeric mixtures ofE- and Z-olefins in complex substrates bearing multiple functionalities. Such a strategy could have value in the synthesis and isolation of natural products, peptides, and pharmaceuticals as even small amounts of stereoisomeric impurities can affect their physical or biological properties. As such, a dual RCM/ethenolysis strategy as a means to control olefin geometry in macrocycles was sought. As a rigorous test of this methodology, the generation of macrocyclic peptides, a class of compounds that are traditionally difficult substrates to synthesize and isolate with defined olefin geometry, was focused on (see Nicolaou, K. C.; Bulger, P. G.; Sarlah, D. Angew. Chem. Int. Ed. 2005, 44, 4490; Gradillas, A.; Perez-Castells, J. Angew. Chem. Int. Ed. 2006, 45, 6086).

Herein, detailed comparative experiments of a variety of ruthenium catalysts in promoting RCM on peptides and the role of catalyst structure in controlling the stereoselectivity of RCM are disclosed. Moreover, through the combined efforts of RCM and catalyst-directed ethenolysis, methods for the selective formation of E- or Z-olefin geometry within macrocyclic peptides are disclosed.

### SUMMARY OF THE DISCLOSURE

In one aspect, the invention discloses the first examples of Z-selective metathesis of peptides using cyclometalated ruthenium catalysts. By examining a broad range of canonical and non-canonical amino acids in cross metathesis, homodimerization, and ring-closing metathesis, important criteria for achieving high conversion while maintaining excellent Z-selectivity are disclosed herein. The following insights based on these results are summarized below.

The side chain identity of an amino acid can dictate the activity of catalysts Ru-1 and Ru-2 in cross metathesis and homodimerization. In general, amino acids bearing aliphatic or aromatic side chains (e.g., alanine, leucine, and phenylalanine) are highly active in metathesis with yields approaching 85% and 94% Z-selectivity. Exceptions include glycine and proline which are inactive in metathesis. Sterically hindered side chains (e.g., valine or isoleucine) and amino acids bearing bulky protecting groups lead to lower conversions but without degradation of Z-selectivity. Amino acids bearing carboxylate functionality (i.e., glutamic acid and aspartic acid) require protection as substrates bearing acidic functionality can lead to catalyst decomposition and diminished Z-selectivity. Amino acids bearing thiols or thioethers generally deactivate cyclometalated ruthenium catalysts, however the use of protecting groups, in some cases, can lead to productive turnovers. Side chains bearing hydroxyl groups (e.g., serine or threonine) are generally tolerated by catalysts Ru-1 and Ru-2 whereas amino acids bearing heterocycles had variable activity. Tryptophan is active in Z-selective homodimerization and cross metathesis, however histidine is generally inactive. Polar side chains bearing carboxamide (i.e., glutamine or asparagine) or guanidinium (i.e., arginine) functionality are generally intolerant of cyclometalated ruthenium catalysts. Protection of these side chains can restore catalyst activity leading to products highly enriched in Z-olefins.

Cross metathesis (CM) and homodimerzation of amino acids and peptides using Z-selective ruthenium catalysts can be performed in a variety of solvents, provided that the acidity of the reaction medium is kept minimal. The use of solvents such as MeCN, DMSO, or DMF generally lead to lower conversions than non-coordinating solvents (e.g., DCE). The use of protic solvents (e.g., MeOH, EtOH, or H₂O) can lead to products enriched in Z-olefins. Prolonged reaction times in protic solvents, in some cases, can lead to decomposition of catalysts Ru-1 and Ru-2.

Amino acids bearing allylic or homoallylic functionality are active in Z-selective metathesis. In general, higher conversions in homodimerization and CM can be achieved using homoallylic functionality, particularly for sterically hindered substrates. Non-canonical amino acids containing allylic heteroatoms including those that could be incorporated into peptides and proteins are active in Z-selective cross metathesis and follow similar trends of non-cyclometalated ruthenium catalysts. The use of aqueous conditions in the presence of salts as additives appears to diminish the activity of catalysts Ru-1 and Ru-2.

Cyclometalated ruthenium catalysts can be used to synthesize stapled peptides bearing hydrocarbon olefinic crosslinks positioned at i, i+4 or i, i+7 residues. To probe the limits of these catalysts in peptide stapling, Z-selective catalysts were exposed to Aib-rich peptides bearing O-allyl serine crosslinks positioned at i, i+3 residues, which predominantly give rise to highly E-selective macrocyclic products. In these cases, catalysts such as Ru-1 failed to undergo Z-selective ring closing metathesis (RCM), suggesting that conformational restrictions imposed by substrates such as 20 (Table 9) can influence the activity of cyclometalated ruthenium catalysts Ru-1 and Ru-2 in RCM.

Notably, compounds 15 (Scheme 3), 17 (Table 8), and 19 (Scheme 4) represent the most complex substrates synthesized by cyclometalated ruthenium catalysts which bodes well for further studies aimed at applying Z-selective metathesis on substrates bearing multiple functionalities. The studies disclosed herein may serve as a guideline in choosing appropriate alkene cross partners or for promoting RCM on peptides. Cyclometalated ruthenium catalysts can be used to access new structures and provide insight into the role of alkene geometry on the biological activity of stapled peptides. Moreover, installation of Z-alkenes into peptides and proteins may allow sites for further modifications. The invention disclosed herein broadens the application of olefin metathesis in natural product synthesis and in biology.

In another aspect, the invention discloses a method for the stereoselective synthesis of macrocyclic peptides using RCM in tandem with catalyst-controlled ethenolysis. The utility of the method was demonstrated on a variety of peptide sequences and olefin crosslinks to enrich macrocycles in E or Z olefin geometry. The strategies outlined herein can facilitate the synthesis and isolation of macrocylic peptides and this approach allowed for the examination of olefin geometry on the conformation of α-helical peptide secondary structures. Notably, a comprehensive evaluation of a variety of ruthenium catalysts in facilitating RCM on peptides is disclosed herein and highlight the use of cyclometalated ruthenium catalysts to control diastereoselectivity in macrocycle synthesis. These studies may enable strategies for accessing novel macrocyclic architectures and may help elucidate the role of olefin geometry on the stability or biological activity of cyclic peptides. The invention disclosed herein broadens the scope and applications of olefin metathesis in areas from chemical biology to natural product synthesis and pharmaceutical development.

In one embodiment the invention provides a method for preparing at least one cross metathesis product, comprising: contacting a first olefin reactant with a second olefin reactant in the presence of a cyclometalated catalyst, under conditions effective to promote the formation of the at least one cross metathesis product; where the first olefin reactant and the second olefin reactant are each independently an optionally substituted amino acid comprising a terminal olefinic moiety or an optionally substituted peptide comprising a terminal olefinic moiety; and where the first olefin reactant and the second olefin reactant are the same or different.

In another embodiment the invention provides a method for preparing a ring-closing metathesis product, comprising: contacting a diolefin reactant with a cyclometalated catalyst under conditions effective to promote the formation of the ring-closing metathesis product; and where the diolefin reactant is an optionally substituted peptide comprising two terminal olefinic moieties.

### DETAILED DESCRIPTION OF THE DISCLOSURE

### Terminology and Definitions

Unless otherwise indicated, the invention is not limited to specific reactants, substituents, catalysts, reaction conditions, or the like, as such may vary. It is also to be understood that the terminology used herein is for the purpose of describing particular embodiments only and is not to be interpreted as being limiting.

As used in the specification and the appended claims, the singular forms “a,” “an,” and “the” include plural referents unless the context clearly dictates otherwise. Thus, for example, reference to “an α-olefin” includes a single α-olefin as well as a combination or mixture of two or more α-olefins, reference to “a substituent” encompasses a single substituent as well as two or more substituents, and the like.

As used in the specification and the appended claims, the terms “for example,” “for instance,” “such as,” or “including” are meant to introduce examples that further clarify more general subject matter. Unless otherwise specified, these examples are provided only as an aid for understanding the invention, and are not meant to be limiting in any fashion.

In this specification and in the claims that follow, reference will be made to a number of terms, which shall be defined to have the following meanings:

The term “alkyl” as used herein refers to a linear, branched, or cyclic saturated hydrocarbon group typically although not necessarily containing 1 to about 24 carbon atoms, preferably 1 to about 12 carbon atoms, such as methyl (Me), ethyl (Et), n-propyl (Pr or n-Pr), isopropyl (i-Pr), n-butyl (Bu or n-Bu), isobutyl (i-Bu), t-butyl (t-Bu), octyl (Oct), decyl, and the like, as well as cycloalkyl groups such as cyclopentyl (Cp), cyclohexyl (Cy) and the like. Generally, although again not necessarily, alkyl groups herein contain 1 to about 12 carbon atoms. The term “lower alkyl” refers to an alkyl group of 1 to 6 carbon atoms, and the specific term “cycloalkyl” refers to a cyclic alkyl group, typically having 4 to 8, preferably 5 to 7, carbon atoms. The term “substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl, respectively.

The term “alkylene” as used herein refers to a difunctional linear, branched, or cyclic alkyl group, where “alkyl” is as defined above.

The term “alkenyl” as used herein refers to a linear, branched, or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Preferred alkenyl groups herein contain 2 to about 12 carbon atoms. The term “lower alkenyl” refers to an alkenyl group of 2 to 6 carbon atoms, and the specific term “cycloalkenyl” refers to a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

An olefinic structure could potentially exist in either cis (Z) or trans (E) configuration, the use of a wavy line in the depiction indicates that the configuration may be either cis or trans or a combination of the two:

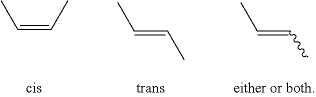

The geometry of the olefins described in this patent application may be (E) or (Z) or a mixture thereof. Applicants have represented a mixture of double-bond isomers by using a squiggly bond “ [symbol] ”.

The term “alkenylene” as used herein refers to a difunctional linear, branched, or cyclic alkenyl group, where “alkenyl” is as defined above.

The term “alkynyl” as used herein refers to a linear or branched hydrocarbon group of 2 to about 24 carbon atoms containing at least one triple bond, such as ethynyl, n-propynyl, and the like. Preferred alkynyl groups herein contain 2 to about 12 carbon atoms. The term “lower alkynyl” refers to an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkynylene” as used herein refers to a difunctional alkynyl group, where “alkynyl” is as defined above.

The term “alkoxy” as used herein refers to an alkyl group bound through a single, terminal ether linkage; that is, an “alkoxy” group may be represented as —O-alkyl where alkyl is as defined above. A “lower alkoxy” group refers to an alkoxy group containing 1 to 6 carbon atoms. Analogously, “alkenyloxy” and “lower alkenyloxy” respectively refer to an alkenyl and lower alkenyl group bound through a single, terminal ether linkage, and “alkynyloxy” and “lower alkynyloxy” respectively refer to an alkynyl and lower alkynyl group bound through a single, terminal ether linkage.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl (Ph), naphthyl, biphenyl, diphenylether, diphenylamine, benzophenone, and the like. “Substituted aryl” refers to an aryl moiety substituted with one or more substituent groups, and the terms “heteroatom containing aryl” and “heteroaryl” refer to aryl substituents in which at least one carbon atom is replaced with a heteroatom, as will be described in further detail herein.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 24 carbon atoms, and particularly preferred aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Preferred alkaryl and aralkyl groups contain 6 to 24 carbon atoms, and particularly preferred alkaryl and aralkyl groups contain 6 to 16 carbon atoms. Alkaryl groups include, without limitation, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The terms “alkaryloxy” and “aralkyloxy” refer to substituents of the formula —OR wherein R is alkaryl or aralkyl, respectively, as just defined.

The term “acyl” refers to substituents having the formula —(CO)-alkyl, —(CO)-aryl, —(CO)-aralkyl, —(CO)-alkaryl, —(CO)-alkenyl, or —(CO)-alkynyl, and the term “acyloxy” refers to substituents having the formula —O(CO)-alkyl, —O(CO)-aryl, —O(CO)-aralkyl, —O(CO)-alkaryl, —O(CO)-alkenyl, or —(CO)-alkynyl wherein “alkyl,” “aryl”, “aralkyl”, “alkaryl”, “alkenyl”, and “alkynyl” are as defined above. The acetoxy group (—O(CO)CH₃; often abbreviated as —OAc) is a common example of an acyloxy group.

The terms “cyclic” and “ring” refer to alicyclic or aromatic groups that may or may not be substituted and/or heteroatom containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic or polycyclic.

The terms “halo” and “halogen” and “halide” are used in the conventional sense to refer to a fluoro, chloro, bromo, or iodo substituent.

“Hydrocarbyl” refers to univalent hydrocarbyl radicals containing 1 to about 30 carbon atoms, preferably 1 to about 24 carbon atoms, most preferably 1 to about 12 carbon atoms, including linear, branched, cyclic, saturated and unsaturated species, such as alkyl groups, alkenyl groups, alkynyl groups, aryl groups, and the like. The term “lower hydrocarbyl” refers to a hydrocarbyl group of 1 to 6 carbon atoms, preferably 1 to 4 carbon atoms, and the term “hydrocarbylene” refers to a divalent hydrocarbyl moiety containing 1 to about 30 carbon atoms, preferably 1 to about 24 carbon atoms, most preferably 1 to about 12 carbon atoms, including linear, branched, cyclic, saturated and unsaturated species. The term “lower hydrocarbylene” refers to a hydrocarbylene group of 1 to 6 carbon atoms. “Substituted hydrocarbyl” refers to hydrocarbyl substituted with one or more substituent groups, and the terms “heteroatom-containing hydrocarbyl” and “heterohydrocarbyl” refer to hydrocarbyl in which at least one carbon atom is replaced with a heteroatom. Similarly, “substituted hydrocarbylene” refers to hydrocarbylene substituted with one or more substituent groups, and the terms “heteroatom-containing hydrocarbylene” and heterohydrocarbylene” refer to hydrocarbylene in which at least one carbon atom is replaced with a heteroatom. Unless otherwise indicated, the term “hydrocarbyl” and “hydrocarbylene” are to be interpreted as including substituted and/or heteroatom-containing hydrocarbyl and hydrocarbylene moieties, respectively.

The term “heteroatom-containing” as in a “heteroatom-containing hydrocarbyl group” refers to a hydrocarbon molecule or a hydrocarbyl molecular fragment in which one or more carbon atoms is replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.” Examples of heteroalkyl groups include without limitation alkoxyaryl, alkylsulfanyl-substituted alkyl, N-alkylated amino alkyl, and the like. Examples of heteroaryl substituents include without limitation pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups include without limitation pyrrolidino, morpholino, piperazino, piperidino, etc.

The term “heterocyclic carbene” refers to a neutral electron donor ligand comprising a carbene molecule, where the carbenic carbon atom is contained within a cyclic structure and where the cyclic structure also contains at least one heteroatom. Examples of heterocyclic carbenes include “N-heterocyclic carbenes” wherein the heteroatom is nitrogen and “P-heterocyclic carbenes” wherein the heteroatom is phosphorus.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation: functional groups referred to herein as “Fn,” such as halo, hydroxyl, sulfhydryl, C₁-C₂₄ alkoxy, C₂-C₂₄ alkenyloxy, C₂-C₂₄ alkynyloxy, C₅-C₂₄ aryloxy, C₆-C₂₄ aralkyloxy, C₆-C₂₄ alkaryloxy, acyl (including C₂-C₂₄ alkylcarbonyl (—CO-alkyl) and C₆-C₂₄ arylcarbonyl (—CO-aryl)), acyloxy (—O-acyl, including C₂-C₂₄ alkylcarbonyloxy (—O—CO-alkyl) and C₆-C₂₄ arylcarbonyloxy (—O—CO-aryl)), C₂-C₂₄ alkoxycarbonyl (—(CO)—O-alkyl), C₆-C₂₄ aryloxycarbonyl (—(CO)—O-aryl), halocarbonyl (—CO)—X where X is halo), C₂-C₂₄ alkylcarbonato (—O—(CO)—O-alkyl), C₆-C₂₄ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-(C₁-C₂₄ haloalkyl)-substituted carbamoyl (—(CO)—NH(C₁-C₂₄ haloalkyl)), di-(C₁-C₂₄ haloalkyl)-substituted carbamoyl (—(CO)—N(C₁-C₂₄ haloalkyl)₂), mono-(C₅-C₂₄ aryl)-substituted carbamoyl (—(CO)—NH-aryl), di-(C₅-C₂₄ aryl)-substituted carbamoyl (—(CO)—N(C₅-C₂₄ aryl)₂), di-N—(C₁-C₂₄ alkyl), N—(C₅-C₂₄ aryl)-substituted carbamoyl (—(CO)—N(C₁-C₂₄ alkyl)(C₅-C₂₄ aryl), thiocarbamoyl (—(CS)—NH₂), mono-(C₁-C₂₄ alkyl)-substituted thiocarbamoyl (—(CS)—NH(C₁-C₂₄ alkyl)), di-(C₁-C₂₄ alkyl)-substituted thiocarbamoyl (—(CS)—N(C₁-C₂₄ alkyl)₂), mono-(C₅-C₂₄ aryl)-substituted thiocarbamoyl (—(CS)—NH-aryl), di-(C₅-C₂₄ aryl)-substituted thiocarbamoyl (—(CS)—N(C₅-C₂₄ aryl)₂), di-N—(C₁-C₂₄ alkyl), N—(C₅-C₂₄ aryl)-substituted thiocarbamoyl (—(CS)—N(C₁-C₂₄ alkyl)(C₅-C₂₄ aryl), carbamido (—NH—(CO)—NH₂), cyano (—C≡N), cyanato (—O—C≡N), thiocyanato (—S—C≡N), isocyanate (—N═C═O), thioisocyanate (—N═C═S), formyl (—(CO)—H), thioformyl (—(CS)—H), amino (—NH₂), mono-(C₁-C₂₄ alkyl)-substituted amino (—NH(C₁-C₂₄ alkyl), di-(C₁-C₂₄ alkyl)-substituted amino ((—N(C₁-C₂₄ alkyl)₂), mono-(C₅-C₂₄ aryl)-substituted amino (—NH(C₅-C₂₄ aryl), di-(C₅-C₂₄ aryl)-substituted amino (—N(C₅-C₂₄ aryl)₂), C₂-C₂₄ alkylamido (—NH—(CO)-alkyl), C₆-C₂₄ arylamido (—NH—(CO)-aryl), imino (—CR═NH where, R includes without limitation hydrogen, C₁-C₂₄ alkyl, C₅-C₂₄ aryl, C₆-C₂₄ alkaryl, C₆-C₂₄ aralkyl, etc.), C₂-C₂₀ alkylimino (—CR═N(alkyl), where R includes without limitation hydrogen, C₁-C₂₄ alkyl, C₅-C₂₄ aryl, C₆-C₂₄ alkaryl, C₆-C₂₄ aralkyl, etc.), arylimino (—CR═N(aryl), where R includes without limitation hydrogen, C₁-C₂₀ alkyl, C₅-C₂₄ aryl, C₆-C₂₄ alkaryl, C₆-C₂₄ aralkyl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), C₅-C₂₄ arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₁-C₂₄ monoalkylaminosulfonyl (—SO₂—N(H) alkyl), C₁-C₂₄ dialkylaminosulfonyl (—SO₂—N(alkyl)₂), C₅-C₂₄ arylsulfonyl (—SO₂-aryl), boryl (—BH₂), borono (—B(OH)₂), boronato (—B(OR)₂ where R includes without limitation alkyl or other hydrocarbyl), phosphono (—P(O)(OH)₂), phosphonato (—P(O)(O⁻)₂), phosphinato (—P(O)(O⁻)), phospho (—PO₂), phosphino (—PH₂), silyl (—SiR₃ wherein R is hydrogen or hydrocarbyl), and silyloxy (—O-silyl); and the hydrocarbyl moieties C₁-C₂₄ alkyl (preferably C₁-C₁₂ alkyl, more preferably C₁-C₆ alkyl), C₂-C₂₄ alkenyl (preferably C₂-C₁₂ alkenyl, more preferably C₂-C₆ alkenyl), C₂-C₂₄ alkynyl (preferably C₂-C₁₂ alkynyl, more preferably C₂-C₆ alkynyl), C₅-C₂₄ aryl (preferably C₅-C₁₄ aryl), C₆-C₂₄ alkaryl (preferably C₆-C₁₆ alkaryl), and C₆-C₂₄ aralkyl (preferably C₆-C₁₆ aralkyl).

By “functionalized” as in “functionalized hydrocarbyl,” “functionalized alkyl,” “functionalized olefin,” “functionalized cyclic olefin,” and the like, is meant that in the hydrocarbyl, alkyl, olefin, cyclic olefin, or other moiety, at least one hydrogen atom bound to a carbon (or other) atom is replaced with one or more functional groups such as those described herein above. The term “functional group” is meant to include any functional species that is suitable for the uses described herein. In particular, as used herein, a functional group may possess the ability to react with or bond to corresponding functional groups on a support surface.

In addition, the aforementioned functional groups may, if a particular group permits, be further substituted with one or more additional functional groups or with one or more hydrocarbyl moieties such as those specifically enumerated above. Analogously, the above mentioned hydrocarbyl moieties may be further substituted with one or more functional groups or additional hydrocarbyl moieties such as those specifically mentioned above. Analogously, the above-mentioned hydrocarbyl moieties may be further substituted with one or more functional groups or additional hydrocarbyl moieties as noted above.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term “nil”, as used herein, means absent or nonexistent.

The term “staple” as used herein refers to the intramolecular or intermolecular connection (also referred to as cross-linking) of two peptides or two peptide residues (e.g., two loops of a helical peptide).

Functional groups may be protected in cases where the functional group interferes with the metathesis catalyst, and any of the protecting groups commonly used in the art may be employed. Acceptable protecting groups may be found, for example, in Greene et al., Protective Groups in Organic Synthesis, 3rd Ed. (New York: Wiley, 1999). Examples of protecting groups include acetals, cyclic acetals, boronate esters (boronates), cyclic boronate esters (cyclic boronates), carbonates, or the like. Examples of protecting groups include cyclic acetals or cyclic boronate esters.

The term “amino acid” as used herein, refers to any naturally occurring amino acid, any unnatural amino acid, or any functionalized amino acid.

The term “peptide” as used herein refers to any combination of two or more amino acids.

The term “naturally occurring amino acids” as used herein, refers to one of any twenty amino acids commonly found in peptides synthesized in nature, known by three letters abbreviations or by one letter abbreviations: Arg (R), His (H), Lys (K), Asp (D), Glu (E), Ser (S), Thr (T), Asn (N), Gln (Q), Cys (C), Gly (G), Pro (P), Ala (A), Val (V), Ile (I), Leu (L), Met (M), Phe (F), Tyr (Y), Trp (W).

The term “unnatural amino acid” as used herein, refers to amino acids which do not occur naturally or which are not found in the genetic code of any organism.

The term “amino acid residue” as used herein refers to an amino acid where the elements of water are removed. α-Amino-acid residues are therefore structures that lack a hydrogen atom of the amino group (—NH—CHR—COOH), or the hydroxyl moiety of the carboxyl group (NH₂—CHR—CO—), or both (—NH—CHR—COO—); all units of a peptide chain are therefore amino-acid residues. The amino acid residue may be derived from any naturally occurring amino acid, any unnatural amino acid, or any functionalized amino acid.

The term “peptide residue” as used herein refers to any combination of two or more amino acid residues.

The term “functionalized amino acid” as used herein, refers to any naturally occurring amino acid or any unnatural amino acid wherein at least one hydrogen atom bound to a carbon (or other) atom is replaced with one or more functional groups such as those described herein.

The term “functionalized peptide” as used herein, refers to any combination of two or more functionalized amino acids.

The term “solid support” as used herein refers to any material that a functional group, a protecting group, an optionally substituted amino acid residue, or an optionally substituted peptide residue may be contacted with, applied to, attached to, or linked to. Examples of solid supports include without limitation, any resin, any type of solid phase, or any type of polymer.

### Cyclometalated Catalysts

In general, the cyclometalated catalysts of the invention comprise a Group 8 metal (M), an alkylidene moiety (═CR¹R²), an anionic ligand (X¹), a neutral ligand (L¹) and a heterocyclic carbene ligand that is linked to the metal via a 2-electron anionic donor bridging moiety (Q*).

The cyclometalated catalysts are preferably a Group 8 transition metal complex and may be represented by the structure of Formula (I):

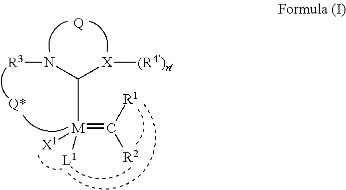

wherein,
M is a Group 8 transition metal (e.g., Ru or Os);
L¹ is a neutral electron donor ligand;
Q* is a 2-electron anionic donor bridging moiety linking R³ and M; and may be hydrocarbylene (including substituted hydrocarbylene, heteroatom-containing hydrocarbylene, and substituted heteroatom-containing hydrocarbylene, such as substituted and/or heteroatom-containing alkylene) or —(CO)—;
Q is a linker, typically a hydrocarbylene linker, including substituted hydrocarbylene, heteroatom-containing hydrocarbylene, and substituted heteroatom-containing hydrocarbylene linkers, wherein two or more substituents on adjacent atoms within Q may also be linked to form an additional cyclic structure, which may be similarly substituted to provide a fused polycyclic structure of two to about five cyclic groups. Q is often, although again not necessarily, a two-atom linkage or a three-atom linkage;
X is an atom selected from C, N, O, S, and P. Since O and S are divalent, n′ is necessarily zero when X is O or S. Similarly, when X is N or P, then n′ is 1, and when X is C, then n′ is 2;
R¹ and R² are independently selected from hydrogen, hydrocarbyl (e.g., C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), substituted hydrocarbyl (e.g., substituted C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), heteroatom-containing hydrocarbyl (e.g., heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and functional groups. R¹ and R² may also be linked to form a cyclic group, which may be aliphatic or aromatic, and may contain substituents and/or heteroatoms. Generally, such a cyclic group will contain 4 to 12, preferably 5, 6, 7, or 8 ring atoms.
R³ and R^{4′} are independently selected from hydrocarbyl, substituted hydrocarbyl, heteroatom-containing hydrocarbyl, and substituted heteroatom-containing, hydrocarbyl (e.g., C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), substituted hydrocarbyl (e.g., substituted C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), heteroatom-containing hydrocarbyl (e.g., heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and functional groups;
X¹ is a bidentate anionic ligand; and
R¹ may connect with R², or R¹ may connect to L¹, or R² may connect to L¹, or L¹ may connect to X¹, to form cyclic groups, these cyclic groups may contain 4 to 12, preferably 4, 5, 6, 7 or 8 atoms, or may comprise two or three of such rings, which may be either fused or linked.

Typically, X¹ is nitrate, C₁-C₂₀ alkylcarboxylate, C₆-C₂₄ arylcarboxylate, C₂-C₂₄ acyloxy, C₁-C₂₀ alkylsulfonato, C₅-C₂₄ arylsulfonato, C₁-C₂₀ alkyl sulfanyl, C₅-C₂₄ arylsulfanyl, C₁-C₂₀ alkylsulfinyl, or C₅-C₂₄ arylsulfinyl. In some embodiments, X¹ is benzoate, pivalate, nitrate, an N-acetyl amino carboxylate, O-methyl mandelate, or a carboxylate derived from 2-phenylbutyric acid. More specifically, X¹ may be is CF₃CO₂, CH₃CO₂, CH₃CH₂CO₂, CFH₂CO₂, (CH₃)₃CO₂, (CH₃)₂CHCO₂, (CF₃)₂(CH₃)CO₂, (CF₃)(CH₃)₂CO₂, benzoate, naphthylate, tosylate, mesylate, or trifluoromethane-sulfonate. In one more preferred embodiment, X¹ is nitrate (NO₃⁻).

In certain catalysts, R¹ is hydrogen and R² is selected from C₁-C₂₀ alkyl, C₂-C₂₀ alkenyl, and C₅-C₂₄ aryl, more preferably C₁-C₆ alkyl, C₂-C₆ alkenyl, and C₅-C₁₄ aryl. In one embodiment, R¹ is hydrogen; and R² is phenyl, which may be optionally substituted with one or more functional groups. Still more preferably, R² is phenyl, vinyl, methyl, isopropyl, or t-butyl, optionally substituted with one or more moieties selected from C₁-C₆ alkyl, C₁-C₆ alkoxy, and phenyl. Most preferably, R² is phenyl or vinyl substituted with one or more moieties selected from methyl, ethyl, chloro, bromo, iodo, fluoro, nitro, dimethylamino, methyl, methoxy, and phenyl. More specifically, R² may be phenyl or —CH═C(CH₃)₂.

Any two or more (typically two, three, or four) of X¹, L¹, R¹, and R² can be taken together to form a cyclic group, including bidentate or multidentate ligands, as disclosed, for example, in U.S. Pat. No. 5,312,940 to Grubbs et al. When any of X¹, L¹, R¹, and R² are linked to form cyclic groups, those cyclic groups may contain 4 to 12, preferably 4, 5, 6, 7 or 8 atoms, or may comprise two or three of such rings, which may be either fused or linked.

In particular embodiments, Q is a two-atom linkage having the structure —CR¹¹R¹²—CR¹³R¹⁴— or —CR¹¹═CR¹³—, preferably —CR¹¹R¹²—CR¹³R¹⁴—, wherein R¹¹, R¹², R¹³, and R¹⁴ are independently selected from hydrogen, hydrocarbyl, substituted hydrocarbyl, heteroatom-containing hydrocarbyl, substituted heteroatom-containing hydrocarbyl, and functional groups. Examples of suitable functional groups include carboxyl, C₁-C₂₀ alkoxy, C₅-C₂₄ aryloxy, C₂-C₂₀ alkoxycarbonyl, C₅-C₂₄ alkoxycarbonyl, C₂-C₂₄ acyloxy, C₁-C₂₀ alkylthio, C₅-C₂₄ arylthio, C₁-C₂₀ alkylsulfonyl, and C₁-C₂₀ alkylsulfinyl, optionally substituted with one or more moieties selected from C₁-C₁₂ alkyl, C₁-C₁₂ alkoxy, C₅-C₁₄ aryl, hydroxyl, sulfhydryl, formyl, and halide. R¹¹, R¹², R¹³, and R¹⁴ are preferably independently selected from hydrogen, C₁-C₁₂ alkyl, substituted C₁-C₁₂ alkyl, C₁-C₁₂ heteroalkyl, substituted C₁-C₁₂ heteroalkyl, phenyl, and substituted phenyl. Alternatively, any two of R¹¹, R¹², R¹³, and R¹⁴ may be linked together to form a substituted or unsubstituted, saturated or unsaturated ring structure, e.g., a C₄-C₁₂ alicyclic group or a C₅ or C₆ aryl group, which may itself be substituted, e.g., with linked or fused alicyclic or aromatic groups, or with other substituents. In one further aspect, any one or more of R¹¹, R¹², R¹³, and R¹⁴ comprises one or more of the linkers.

In more particular aspects, R³ and R^{4′} maybe alkyl or aryl, and may be independently selected from alkyl, aryl, cycloalkyl, heteroalkyl, alkenyl, alkynyl, and halo or halogen-containing groups. More specifically, R³ and R⁴ may be independently selected from C₁-C₂₀ alkyl, C₅-C₁₄ cycloalkyl, C₁-C₂₀ heteroalkyl, or halide. Suitable alkyl groups include, without limitation, methyl, ethyl, n-propyl, isopropyl, isopropyl, n-butyl, isobutyl, t-butyl, octyl, decyl, and the like; suitable cycloalkyl groups include cyclopentyl, cyclohexyl, adamantyl, pinenyl, terpenes and terpenoid derivatives and the like; suitable alkenyl groups include ethenyl, n-propenyl, isopropenyl, n-butenyl, isobutenyl, octenyl, decenyl, tetradecenyl, hexadecenyl, eicosenyl, tetracosenyl, and the like; suitable alkynyl groups include ethynyl, n-propynyl, and the like.

When R³ or R^{4′} are aromatic, each can be independently composed of one or two aromatic rings, which may or may not be substituted, e.g., R³ and R^{4′} may be phenyl, substituted phenyl, biphenyl, substituted biphenyl, or the like. In a particular embodiment, R³ and R⁴ are independently an unsubstituted phenyl or phenyl substituted with up to three substituents selected from C₁-C₂₀ alkyl, C₁-C₂₀ alkylcarboxylate, substituted C₁-C₂₀ alkyl, C₁-C₂₀ heteroalkyl, substituted C₁-C₂₀ heteroalkyl, C₅-C₂₄ aryl, substituted C₅-C₂₄ aryl, C₅-C₂₄ heteroaryl, C₆-C₂₄ aralkyl, C₆-C₂₄ alkaryl, or halide. Preferably, any substituents present are hydrogen C₁-C₁₂ alkyl, C₁-C₁₂ alkoxy, C₅-C₁₄ aryl, substituted, C₅-C₁₄ aryl, or halide. More particularly, R³ and R⁴ may be independently substituted with hydrogen, C₁-C₄ alkyl, C₁-C₄ alkylcarboxylate, C₁-C₄ alkoxy, C₅-C₁₄ aryl, substituted C₅-C₁₄ aryl, or halide. As an example, R³ and R^{4′} are selected from cyclopentyl, cyclohexyl, adamantyl, norbonenyl, pinenyl, terpenes and terpenoid derivatives, mesityl, diisopropylphenyl or, more generally, cycloalkyl substituted with one, two or three C₁-C₄ alkyl or C₁-C₄ alkoxy groups, or a combination thereof.

In another aspect, the cyclometalated catalysts of the invention may be represented by the structure of Formula (II):

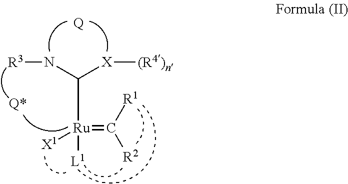

wherein: X, X¹, Q, Q*, R³, R^{4′} and n′ are as defined previously for Formula (I).

Particular complexes wherein R² and L¹ are linked to form a chelating carbene ligand are examples of another group of cyclometalated catalysts, and may be represented by the structure of Formula (III):

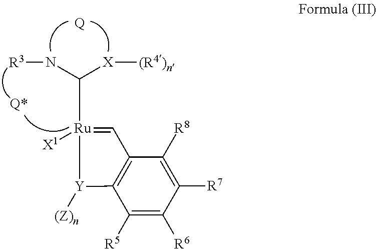

wherein,
X, X¹, Q, Q*, R³, R^{4′} and n′ are as defined previously for Formula (I);
Y is a heteroatom selected from N, O, S, and P; preferably Y is O or N;
R⁵, R⁶, R⁷, and R⁸ are each, independently, selected from the group consisting of hydrogen, halogen, alkyl, alkenyl, alkynyl, aryl, heteroalkyl, heteroatom containing alkenyl, heteroalkenyl, heteroaryl, alkoxy, alkenyloxy, aryloxy, alkoxycarbonyl, carbonyl, alkylamino, alkylthio, aminosulfonyl, monoalkylaminosulfonyl, dialkylaminosulfonyl, alkylsulfonyl, nitrile, nitro, alkylsulfinyl, trihaloalkyl, perfluoroalkyl, carboxylic acid, ketone, aldehyde, nitrate, cyano, isocyanate, hydroxyl, ester, ether, amine, imine, amide, halogen-substituted amide, trifluoroamide, sulfide, disulfide, sulfonate, carbamate, silane, siloxane, phosphine, phosphate, or borate, wherein any combination of R⁵, R⁶, R⁷, and R⁸ can be linked to form one or more cyclic groups;
n is 1 or 2, such that n is 1 when Y is the divalent heteroatoms O or S, and n is 2 when Y is the trivalent heteroatoms N or P; and
Z is a group selected from hydrogen, alkyl, aryl, functionalized alkyl, functionalized aryl where the functional group(s) may independently be one or more or the following: alkoxy, aryloxy, halogen, carboxylic acid, ketone, aldehyde, nitrate, cyano, isocyanate, hydroxyl, ester, ether, amine, imine, amide, trifluoroamide, sulfide, disulfide, carbamate, silane, siloxane, phosphine, phosphate, or borate; methyl, isopropyl, sec-butyl, t-butyl, neopentyl, benzyl, phenyl and trimethylsilyl; and wherein any combination or combinations of X¹, Q*, Y, Z, R⁵, R⁶, R⁷, and R⁸ may be optionally linked to a support.

In another embodiment, the cyclometalated catalysts of the invention may be represented by the structure of Formula (IV):

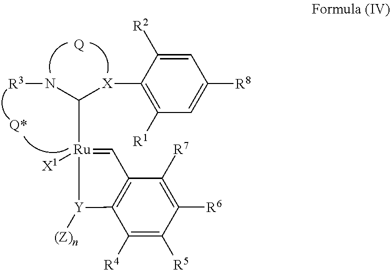

wherein,
R¹ is hydrogen, C₁-C₆ alkyl, substituted C₁-C₆ alkyl, C₁-C₆ heteroalkyl, substituted C₁-C₆ heteroalkyl; C₅-C₂₄ aryl, substituted C₅-C₂₄ aryl; C₅-C₂₄ heteroaryl, substituted C₅-C₂₄ heteroaryl; C₁-C₆ alkoxy, C₆-C₂₄ aralkyl, substituted C₆-C₂₄ aralkyl; C₆-C₂₄ alkaryl, substituted C₆-C₂₄ alkaryl, or halide where the substituents are selected from C₁-C₆ alkyl, C₁-C₆ alkoxy, and halide; in other embodiments R¹ is hydrogen, C₁-C₄ alkyl, C₁-C₄ alkylcarboxylate, C₁-C₄ alkoxy, C₅-C₁₄ aryl, substituted C₅-C₁₄ aryl, or halide; in other embodiments R¹ is C₁-C₆ alkyl, or F; in other embodiments R¹ is C₁-C₃ alkyl, C₁-C₃ alkoxy, or F; in other embodiments R¹ is C₁-C₄ alkyl or F; in other embodiments R¹ is C₁-C₃ alkyl or F; in other embodiments R¹ is OCH₃ (i.e. OMe); in another embodiment R¹ is hydrogen or C₁-C₃ alkyl; in another embodiment R¹ is C₁-C₃ alkyl;
R² is hydrogen, C₁-C₆ alkyl, substituted C₁-C₆ alkyl, C₁-C₆ heteroalkyl, substituted C₁-C₆ heteroalkyl; C₅-C₂₄ aryl, substituted C₅-C₂₄ aryl; C₅-C₂₄ heteroaryl, substituted C₅-C₂₄ heteroaryl; C₁-C₆ alkoxy, C₆-C₂₄ aralkyl, substituted C₆-C₂₄ aralkyl; C₆-C₂₄ alkaryl, substituted C₆-C₂₄ alkaryl, or halide, where the substituents are selected from C₁-C₆ alkyl, C₁-C₆ alkoxy, and halide; in other embodiments R² is hydrogen, C₁-C₄ alkyl, C₁-C₄ alkylcarboxylate, C₁-C₄ alkoxy, C₅-C₁₄ aryl, substituted C₅-C₁₄ aryl, or halide; in other embodiments R² is C₁-C₆ alkyl, or F; in other embodiments R² is C₁-C₃ alkyl, C₁-C₃ alkoxy, or F; in other embodiments R² is C₁-C₄ alkyl or F; in other embodiments R² is C₁-C₃ alkyl or F; in other embodiments R² is OCH₃ (i.e. OMe); in another embodiment R² is hydrogen or C₁-C₃ alkyl; in another embodiment R² is C₁-C₃ alkyl;
R⁸ is selected from hydrogen, C₁-C₁₀ alkyl, substituted C₁-C₁₀ alkyl, C₅-C₁₀ aryl, substituted C₅-C₁₀ aryl, C₅-C₁₀ heteroaryl, substituted C₅-C₁₀ heteroaryl, halide (—Cl, —F, —Br, —I), hydroxyl, C₁-C₆ alkoxy, C₅-C₁₀ aryloxy, nitro (—NO₂), ester (—COOR⁹), ketone (—COR⁹), aldehyde (—COH), acyl (—COR⁹), ester (—OCOR⁹), carboxylic acid (—COOH), sulfonamide (—NR⁹SO₂Ar), carbamate (—NCO₂R⁹), cyano (—CN), sulfoxide (—SOR⁹), sulfonyl (—SO₂R⁹), sulfonic acid (—SO₃H), fluoromethyl (—CFₘ), fluroaryl (e.g., —C₆F₅, p-CF₃C₆H₄), where R⁹ is hydrogen, methyl, C₂-C₆ alkyl, substituted C₂-C₆ alkyl, C₅-C₁₀ aryl, or substituted C₅-C₁₀ aryl, wherein m is 1, 2, or 3; in another embodiment R⁸ is selected from hydrogen, C₁-C₁₀ alkyl, C₅-C₁₀ aryl, C₅-C₁₀ heteroaryl, halide (—Cl, —F, —Br, —I), C₁-C₆ alkoxy, C₅-C₁₀ aryloxy, nitro (—NO₂), ester (—COOR⁹), ketone (—COR⁹), aldehyde (—COH), acyl (—COR⁹), ester (—OCOR⁹), carboxylic acid (—COOH), sulfonamide (—NR⁹SO₂Ar), carbamate (—NCO₂R⁹), cyano (—CN), sulfoxide (—SOR⁹), sulfonyl (—SO₂R⁹), sulfonic acid (—SO₃H), fluoromethyl (—CFₘ), fluroaryl (e.g., —C₆F₅, p-CF₃C₆H₄), where R⁹ is hydrogen, methyl, C₂-C₆ alkyl, substituted C₂-C₆ alkyl, C₅-C₁₀ aryl, or substituted C₅-C₁₀ aryl, wherein m is 1, 2, or 3; in another embodiment R⁸ is selected from hydrogen, C₁-C₁₀ alkyl, halide (—Cl, —F, —Br, —I), C₁-C₆ alkoxy, nitro (—NO₂), ester (—COOR⁹), ketone (—COR⁹), aldehyde (—COH), acyl (—COR⁹), ester (—OCOR⁹), carboxylic acid (—COOH), carbamate (—NCO₂R⁹), cyano (—CN), sulfoxide (—SOR⁹), sulfonyl (—SO₂R⁹), sulfonic acid (—SO₃H), fluoromethyl (—CFₘ), fluroaryl (e.g., —C₆F₅, p-CF₃C₆H₄), where R⁹ is hydrogen, methyl, C₂-C₆ alkyl, wherein m is 1, 2, or 3; in another embodiment R⁸ is selected from hydrogen, C₁-C₁₀ alkyl, halide (—Cl, —F, —Br, —I), C₁-C₆ alkoxy, nitro (—NO₂), ester (—COOR⁹), ketone (—COR⁹), aldehyde (—COH), acyl (—COR⁹), ester (—OCOR⁹), cyano (—CN), where R⁹ is hydrogen, methyl, C₂-C₆ alkyl; in another embodiment R⁸ is selected from hydrogen and C₁-C₃ alkyl;
Q* is a 2-electron anionic donor bridging moiety linking R³ and Ru; and may be hydrocarbylene (including substituted hydrocarbylene, heteroatom-containing hydrocarbylene, and substituted heteroatom-containing hydrocarbylene, such as substituted and/or heteroatom-containing alkylene) or —(CO)—;
Q is a linker, typically a hydrocarbylene linker, including substituted hydrocarbylene, heteroatom-containing hydrocarbylene, and substituted heteroatom-containing hydrocarbylene linkers, wherein two or more substituents on adjacent atoms within Q may also be linked to form an additional cyclic structure, which may be similarly substituted to provide a fused polycyclic structure of two to about five cyclic groups. Q is often, although again not necessarily, a two-atom linkage or a three-atom linkage. In particular embodiments, Q is a two-atom linkage having the structure —CR¹¹R¹²—CR¹³R¹⁴— or —CR¹¹═CR¹³—, preferably —CR¹¹R¹²—CR¹³R¹⁴—, wherein R¹¹, R¹², R¹³, and R¹⁴ are independently selected from hydrogen, hydrocarbyl, substituted hydrocarbyl, heteroatom-containing hydrocarbyl, substituted heteroatom-containing hydrocarbyl, and functional groups. Examples of suitable functional groups include carboxyl, C₁-C₂₀ alkoxy, C₅-C₂₄ aryloxy, C₂-C₂₀ alkoxycarbonyl, C₅-C₂₄ alkoxycarbonyl, C₂-C₂₄ acyloxy, C₁-C₂₀ alkylthio, C₅-C₂₄ arylthio, C₁-C₂₀ alkylsulfonyl, and C₁-C₂₀ alkylsulfinyl, optionally substituted with one or more moieties selected from C₁-C₁₂ alkyl, C₁-C₁₂ alkoxy, C₅-C₁₄ aryl, hydroxyl, sulfhydryl, formyl, and halide. R¹¹, R¹², R¹³, and R¹⁴ are preferably independently selected from hydrogen, C₁-C₁₂ alkyl, substituted C₁-C₁₂ alkyl, C₁-C₁₂ heteroalkyl, substituted C₁-C₁₂ heteroalkyl, phenyl, and substituted phenyl. Alternatively, any two of R¹¹, R¹², R¹³, and R¹⁴ may be linked together to form a substituted or unsubstituted, saturated or unsaturated ring structure, e.g., a C₄-C₁₂ alicyclic group or a C₅ or C₆ aryl group, which may itself be substituted, e.g., with linked or fused alicyclic or aromatic groups, or with other substituents. In one further aspect, any one or more of R¹¹, R¹², R¹³, and R¹⁴ comprises one or more of the linkers;
X is an atom selected from C, N, and P; in one embodiment X is an atom selected from N;
R³ is independently selected from hydrocarbyl, substituted hydrocarbyl, heteroatom-containing hydrocarbyl, and substituted heteroatom-containing, hydrocarbyl (e.g., C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), substituted hydrocarbyl (e.g., substituted C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), heteroatom-containing hydrocarbyl (e.g., heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and functional groups;
X¹ is a bidentate anionic ligand; in one embodiment X¹ is nitrate (NO₃⁻), C₁-C₂₀ alkylcarboxylate, C₆-C₂₄ arylcarboxylate, C₂-C₂₄ acyloxy, C₁-C₂₀ alkylsulfonato, C₅-C₂₄ arylsulfonato, C₁-C₂₀ alkylsulfanyl, C₅-C₂₄ arylsulfanyl, C₁-C₂₀ alkylsulfinyl, or C₅-C₂₄ arylsulfinyl; in another embodiment X¹ is benzoate, pivalate, nitrate (NO₃⁻), an N-acetyl amino carboxylate, O-methyl mandelate, or a carboxylate derived from 2-phenylbutyric acid; in another embodiment X¹ is CF₃CO₂, CH₃CO₂, CH₃CH₂CO₂, CFH₂CO₂, (CH₃)₃CO₂, (CH₃)₂CHCO₂, (CF₃)₂(CH₃)CO₂, (CF₃)(CH₃)₂CO₂, benzoate, naphthylate, tosylate, mesylate, or trifluoromethane-sulfonate; in another embodiment, X¹ is pivalate or nitrate (NO₃⁻); in another embodiment, X¹ is nitrate (NO₃⁻);
Y is a heteroatom selected from N, O, S, and P; in another embodiment Y is a heteroatom selected from O or N; in another embodiment Y is a heteroatom selected from O;
R⁴, R⁵, R⁶, and R⁷ are each, independently, selected from hydrogen, halogen, alkyl, alkenyl, alkynyl, aryl, heteroalkyl, heteroatom containing alkenyl, heteroalkenyl, heteroaryl, alkoxy, alkenyloxy, aryloxy, alkoxycarbonyl, carbonyl, alkylamino, alkylthio, aminosulfonyl, monoalkylaminosulfonyl, dialkylaminosulfonyl, alkylsulfonyl, nitrile, nitro, alkylsulfinyl, trihaloalkyl, perfluoroalkyl, carboxylic acid, ketone, aldehyde, nitrate, cyano, isocyanate, hydroxyl, ester, ether, amine, imine, amide, halogen-substituted amide, trifluoroamide, sulfide, disulfide, sulfonate, sulfonamide, carbamate, silane, siloxane, phosphine, phosphate, or borate, wherein any combination of R⁴, R⁵, R⁶, and R⁷ can be linked to form one or more cyclic groups;
n is 1 or 2, such that n is 1 when Y is the divalent heteroatoms O or S, and n is 2 when Y is the trivalent heteroatoms N or P; and
Z is a group selected from hydrogen, alkyl, aryl, functionalized alkyl, functionalized aryl where the functional group(s) may independently be one or more or the following: alkoxy, aryloxy, halogen, carboxylic acid, ketone, aldehyde, nitrate, cyano, isocyanate, hydroxyl, ester, ether, amine, imine, amide, trifluoroamide, sulfide, disulfide, carbamate, silane, siloxane, phosphine, phosphate, or borate; methyl, isopropyl, sec-butyl, t-butyl, neopentyl, benzyl, phenyl and trimethylsilyl; and wherein any combination or combinations of X¹, Q*, Y, Z, R⁴, R⁵, R⁶, and R⁷ may be optionally linked to a support; in one embodiment Z is selected from C₁-C₆ alkyl; in one embodiment Z is selected from C₁-C₃ alkyl.

In a further embodiment, for the cyclometalated catalysts of the invention represented by the structure of Formula (IV), R³ maybe alkyl or aryl, and may be independently selected from alkyl, aryl, cycloalkyl, heteroalkyl, alkenyl, alkynyl, and halo or halogen-containing groups. In one embodiment, R³ is selected from C₁-C₂₀ alkyl, C₅-C₁₄ cycloalkyl, C₁-C₂₀ heteroalkyl, or halide. Suitable alkyl groups include, without limitation, methyl, ethyl, n-propyl, isopropyl, isopropyl, n-butyl, isobutyl, t-butyl, octyl, decyl, and the like; suitable cycloalkyl groups include cyclopentyl, cyclohexyl, adamantyl, pinenyl, terpenes and terpenoid derivatives and the like; suitable alkenyl groups include ethenyl, n-propenyl, isopropenyl, n-butenyl, isobutenyl, octenyl, decenyl, tetradecenyl, hexadecenyl, eicosenyl, tetracosenyl, and the like; suitable alkynyl groups include ethynyl, n-propynyl, and the like; in one embodiment R³ is selected from t-butyl or adamantyl; in one embodiment R³ is selected from adamantyl.

In one embodiment, for the cyclometalated catalysts of the invention represented by the structure of Formula (IV), when R³ is aromatic, each can be independently composed of one or two aromatic rings, which may or may not be substituted, e.g., R³ may be phenyl, substituted phenyl, biphenyl, substituted biphenyl, or the like. In a particular embodiment, R³ is an unsubstituted phenyl or phenyl substituted with up to three substituents selected from C₁-C₂₀ alkyl, C₁-C₂₀ alkylcarboxylate, substituted C₁-C₂₀ alkyl, C₁-C₂₀ heteroalkyl, substituted C₁-C₂₀ heteroalkyl, C₅-C₂₄ aryl, substituted C₅-C₂₄ aryl, C₅-C₂₄ heteroaryl, C₆-C₂₄ aralkyl, C₆-C₂₄ alkaryl, or halide. In one embodiment, any substituents present are hydrogen C₁-C₁₂ alkyl, C₁-C₁₂ alkoxy, C₅-C₁₄ aryl, substituted, C₅-C₁₄ aryl, or halide. In another embodiment, R³ is substituted with hydrogen, C₁-C₄ alkyl, C₁-C₄ alkylcarboxylate, C₁-C₄ alkoxy, C₅-C₁₄ aryl, substituted C₅-C₁₄ aryl, or halide.

As an example, for the cyclometalated catalysts of the invention represented by the structure of Formula (IV), R³ is selected from cyclopentyl, cyclohexyl, adamantyl, norbonenyl, pinenyl, terpenes and terpenoid derivatives, mesityl, diisopropylphenyl or, more generally, cycloalkyl substituted with one, two or three C₁-C₄ alkyl or C₁-C₄ alkoxy groups, or a combination thereof. In one embodiment, R³ is selected from mesityl, t-butyl, or adamantyl. In one embodiment, R³ is selected from mesityl or adamantyl.

Still, in a further embodiment, the cyclometalated catalysts of the invention may be represented by the structure of Formula (V):

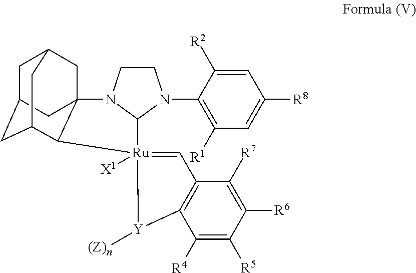

wherein,
R¹ is hydrogen, C₁-C₆ alkyl, substituted C₁-C₆ alkyl, C₁-C₆ heteroalkyl, substituted C₁-C₆ heteroalkyl; C₅-C₂₄ aryl, substituted C₅-C₂₄ aryl; C₅-C₂₄ heteroaryl, substituted C₅-C₂₄ heteroaryl; C₁-C₆ alkoxy, C₆-C₂₄ aralkyl, substituted C₆-C₂₄ aralkyl; C₆-C₂₄ alkaryl, substituted C₆-C₂₄ alkaryl, or halide, where the substituents are selected from C₁-C₆ alkyl, C₁-C₆ alkoxy, and halide; in other embodiments R¹ is hydrogen, C₁-C₄ alkyl, C₁-C₄ alkylcarboxylate, C₁-C₄ alkoxy, C₅-C₁₄ aryl, substituted C₅-C₁₄ aryl, or halide; in other embodiments R¹ is C₁-C₆ alkyl, or F; in other embodiments R¹ is C₁-C₃ alkyl, C₁-C₃ alkoxy, or F; in other embodiments R¹ is C₁-C₄ alkyl or F; in other embodiments R¹ is C₁-C₃ alkyl or F; in other embodiments R¹ is OCH₃ (i.e. OMe); in another embodiment R¹ is hydrogen or C₁-C₃ alkyl; in another embodiment R¹ is C₁-C₃ alkyl;
R² is hydrogen, C₁-C₆ alkyl, substituted C₁-C₆ alkyl, C₁-C₆ heteroalkyl, substituted C₁-C₆ heteroalkyl; C₅-C₂₄ aryl, substituted C₅-C₂₄ aryl; C₅-C₂₄ heteroaryl, substituted C₅-C₂₄ heteroaryl; C₁-C₆ alkoxy, C₆-C₂₄ aralkyl, substituted C₆-C₂₄ aralkyl; C₆-C₂₄ alkaryl, substituted C₆-C₂₄ alkaryl, or halide, where the substituents are selected from C₁-C₆ alkyl, C₁-C₆ alkoxy, and halide; in other embodiments R² is hydrogen, C₁-C₄ alkyl, C₁-C₄ alkylcarboxylate, C₁-C₄ alkoxy, C₅-C₁₄ aryl, substituted C₅-C₁₄ aryl, or halide; in other embodiments R² is C₁-C₆ alkyl, or F; in other embodiments R² is C₁-C₃ alkyl, C₁-C₃ alkoxy, or F; in other embodiments R² is C₁-C₄ alkyl or F; in other embodiments R² is C₁-C₃ alkyl or F; in other embodiments R² is OCH₃ (i.e. OMe); in another embodiment R² is hydrogen or C₁-C₃ alkyl; in another embodiment R² is C₁-C₃ alkyl;
R⁸ is selected from hydrogen, C₁-C₁₀ alkyl, substituted C₁-C₁₀ alkyl, C₅-C₁₀ aryl, substituted C₅-C₁₀ aryl, C₅-C₁₀ heteroaryl, substituted C₅-C₁₀ heteroaryl, halide (—Cl, —F, —Br, —I), hydroxyl, C₁-C₆ alkoxy, C₅-C₁₀ aryloxy, nitro (—NO₂), ester (—COOR⁹), ketone (—COR⁹), aldehyde (—COH), acyl (—COR⁹), ester (—OCOR⁹), carboxylic acid (—COOH), sulfonamide (—NR⁹SO₂Ar), carbamate (—NCO₂R⁹), cyano (—CN), sulfoxide (—SOR⁹), sulfonyl (—SO₂R⁹), sulfonic acid (—SO₃H), fluoromethyl (—CFₘ), fluroaryl (e.g., —C₆F₅, p-CF₃C₆H₄), where R⁹ is hydrogen, methyl, C₂-C₆ alkyl, substituted C₂-C₆ alkyl, C₅-C₁₀ aryl, or substituted C₅-C₁₀ aryl, wherein m is 1, 2, or 3; in another embodiment R⁸ is selected from hydrogen, C₁-C₁₀ alkyl, C₅-C₁₀ aryl, C₅-C₁₀ heteroaryl, halide (—Cl, —F, —Br, —I), C₁-C₆ alkoxy, C₅-C₁₀ aryloxy, nitro (—NO₂), ester (—COOR⁹), ketone (—COR⁹), aldehyde (—COH), acyl (—COR⁹), ester (—OCOR⁹), carboxylic acid (—COOH), sulfonamide (—NR⁹SO₂Ar), carbamate (—NCO₂R⁹), cyano (—CN), sulfoxide (—SOR⁹), sulfonyl (—SO₂R⁹), sulfonic acid (—SO₃H), fluoromethyl (—CFₘ), fluroaryl (e.g., —C₆F₅, p-CF₃C₆H₄), where R⁹ is hydrogen, methyl, C₂-C₆ alkyl, substituted C₂-C₆ alkyl, C₅-C₁₀ aryl, or substituted C₅-C₁₀ aryl, wherein m is 1, 2, or 3; in another embodiment R⁸ is selected from hydrogen, C₁-C₁₀ alkyl, halide (—Cl, —F, —Br, —I), C₁-C₆ alkoxy, nitro (—NO₂), ester (—COOR⁹), ketone (—COR⁹), aldehyde (—COH), acyl (—COR⁹), ester (—OCOR⁹), carboxylic acid (—COOH), carbamate (—NCO₂R⁹), cyano (—CN), sulfoxide (—SOR⁹), sulfonyl (—SO₂R⁹), sulfonic acid (—SO₃H), fluoromethyl (—CFₘ), fluroaryl (e.g., —C₆F₅, p-CF₃C₆H₄), where R⁹ is hydrogen, methyl, C₂-C₆ alkyl, wherein m is 1, 2, or 3; in another embodiment R⁸ is selected from hydrogen, C₁-C₁₀ alkyl, halide (—Cl, —F, —Br, —I), C₁-C₆ alkoxy, nitro (—NO₂), ester (—COOR⁹), ketone (—COR⁹), aldehyde (—COH), acyl (—COR⁹), ester (—OCOR⁹), cyano (—CN), where R⁹ is hydrogen, methyl, C₂-C₆ alkyl; in another embodiment R⁸ is selected from hydrogen and C₁-C₃ alkyl;
X¹ is a bidentate anionic ligand; in one embodiment X¹ is nitrate (NO₃⁻), C₁-C₂₀ alkylcarboxylate, C₆-C₂₄ arylcarboxylate, C₂-C₂₄ acyloxy, C₁-C₂₀ alkylsulfonato, C₅-C₂₄ arylsulfonato, C₁-C₂₀ alkylsulfanyl, C₅-C₂₄ arylsulfanyl, C₁-C₂₀ alkylsulfinyl, or C₅-C₂₄ arylsulfinyl; in another embodiment X¹ is benzoate, pivalate, nitrate (NO₃⁻), an N-acetyl amino carboxylate, O-methyl mandelate, or a carboxylate derived from 2-phenylbutyric acid; in another embodiment X¹ is CF₃CO₂, CH₃CO₂, CH₃CH₂CO₂, CFH₂CO₂, (CH₃)₃CO₂, (CH₃)₂CHCO₂, (CF₃)₂(CH₃)CO₂, (CF₃)(CH₃)₂CO₂, benzoate, naphthylate, tosylate, mesylate, or trifluoromethane-sulfonate; in another embodiment, X¹ is pivalate or nitrate (NO₃⁻); in another embodiment, X¹ is nitrate (NO₃⁻);
Y is a heteroatom selected from N, O, S, and P; in another embodiment Y is a heteroatom selected from O or N; in another embodiment Y is a heteroatom selected from O;
R⁴, R⁵, R⁶, and R⁷ are each, independently, selected from hydrogen, halogen, alkyl, alkenyl, alkynyl, aryl, heteroalkyl, heteroatom containing alkenyl, heteroalkenyl, heteroaryl, alkoxy, alkenyloxy, aryloxy, alkoxycarbonyl, carbonyl, alkylamino, alkylthio, aminosulfonyl, monoalkylaminosulfonyl, dialkylaminosulfonyl, alkylsulfonyl, nitrile, nitro, alkylsulfinyl, trihaloalkyl, perfluoroalkyl, carboxylic acid, ketone, aldehyde, nitrate, cyano, isocyanate, hydroxyl, ester, ether, amine, imine, amide, halogen-substituted amide, trifluoroamide, sulfide, disulfide, sulfonate, sulfonamide, carbamate, silane, siloxane, phosphine, phosphate, or borate, wherein any combination of R⁴, R⁵, R⁶, and R⁷ can be linked to form one or more cyclic groups;
n is 1 or 2, such that n is 1 when Y is the divalent heteroatoms O or S, and n is 2 when Y is the trivalent heteroatoms N or P; and
Z is a group selected from hydrogen, alkyl, aryl, functionalized alkyl, functionalized aryl where the functional group(s) may independently be one or more or the following: alkoxy, aryloxy, halogen, carboxylic acid, ketone, aldehyde, nitrate, cyano, isocyanate, hydroxyl, ester, ether, amine, imine, amide, trifluoroamide, sulfide, disulfide, carbamate, silane, siloxane, phosphine, phosphate, or borate; methyl, isopropyl, sec-butyl, t-butyl, neopentyl, benzyl, phenyl and trimethylsilyl; and wherein any combination or combinations of X¹, Q*, Y, Z, R⁴, R⁵, R⁶, and R⁷ may be optionally linked to a support; in one embodiment Z is selected from C₁-C₆ alkyl; in one embodiment Z is selected from C₁-C₃ alkyl.

Examples of cyclometalated catalysts represented by the structure of Formula (V) include the following:

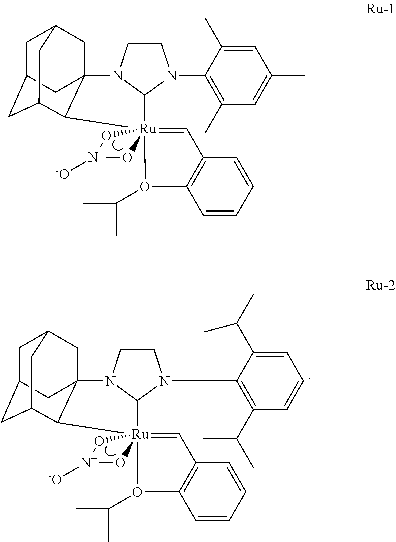

Examples of cyclometalated catalysts represented by the structure of Formula (V) include the following:

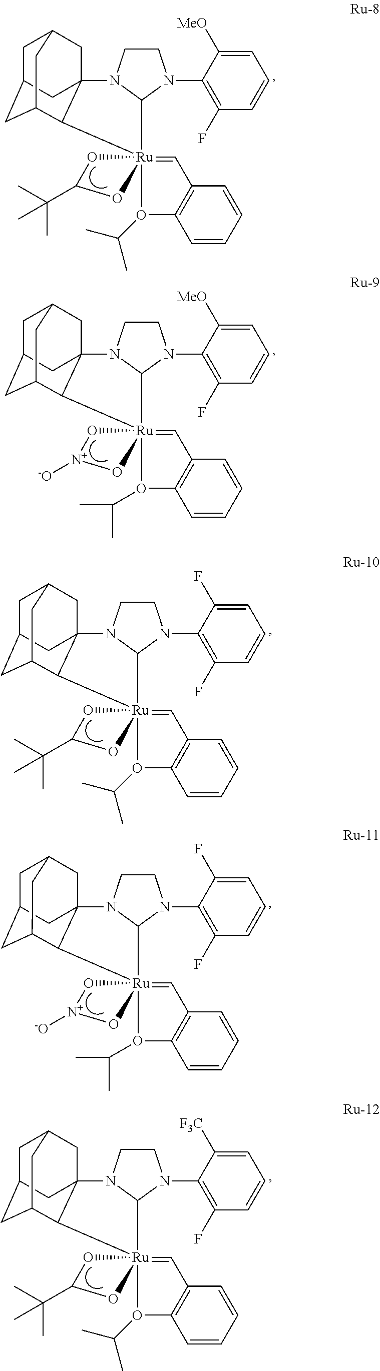

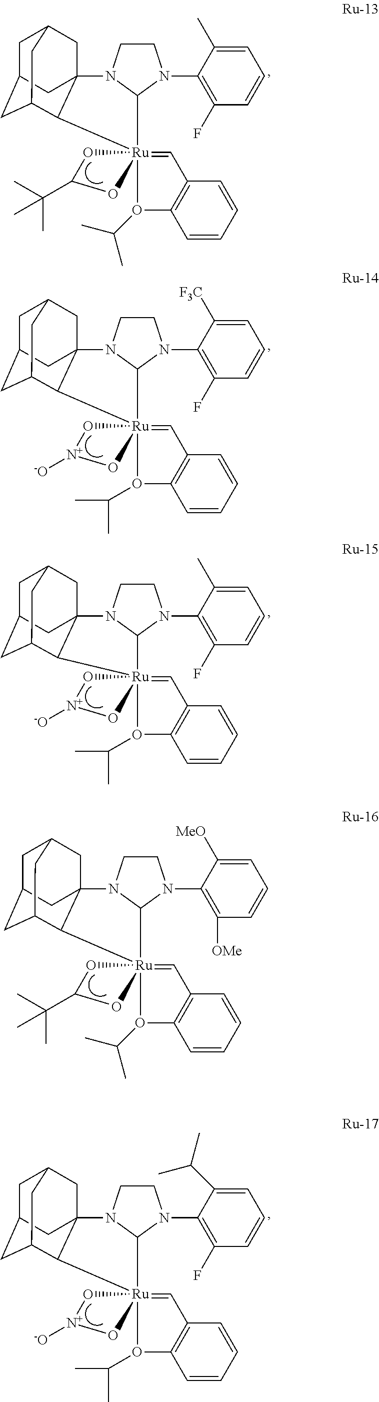

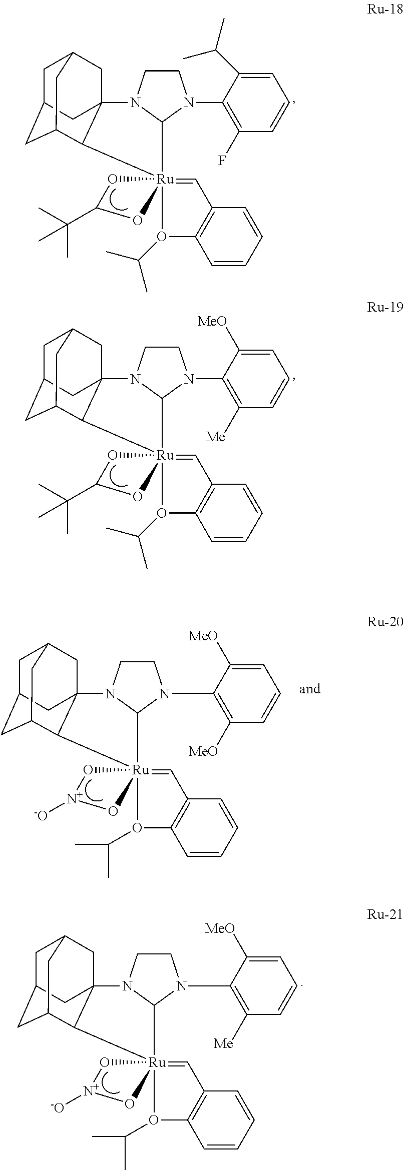

In another aspect the invention can be practiced in the presence of Schrock and Hoveyda, (see Flook, M. M.; Jiang, A. J.; Schrock, R. R.; Müller, P.; Hoveyda, A. H. J. Am. Chem. Soc. 2009, 131, 7962-7963. Jiang, A. J.; Zhao, Y.; Schrock, R. R.; Hoveyda, A. H. J. Am. Chem. Soc. 2009, 131, 16630-16631; Yu, M.; Wang, C.; Kyle, A. F.; Jukubec, P.; Dixon, D. J.; Schrock, R. R.; Hoveyda, A. H. Nature 2011, 479, 88-93; Meek, S. J.; O'Brien, R. V.; Llaveria, J.; Schrock, R. R.; Hoveyda, A. H. Nature 2011, 471, 461-466; Speed, A. W. H.; Mann, T. J.; O'Brien, R. V.; Schrock, R. R.; Hoveyda, A. H. J. Am. Chem. Soc. 2014, 136, 16136-16139) complexes of molybdenum and tungsten such as:

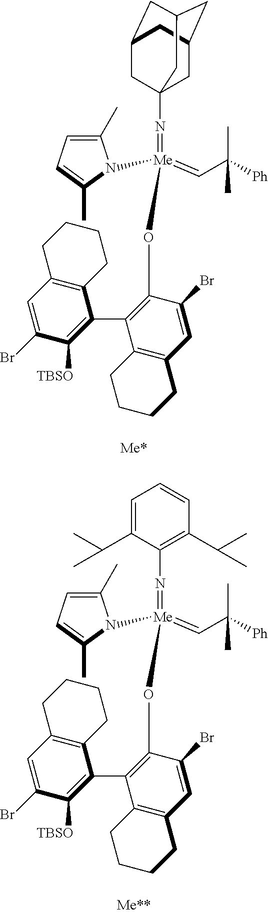

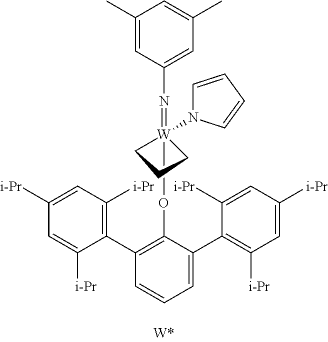

### Olefin Reactants

In one embodiment, the olefin reactant is an optionally substituted amino acid comprising a terminal olefinic moiety en, a solid support, an optionally substituted peptide comprising a terminal olefinic moiety.

In one embodiment, the olefin reactant is an optionally substituted amino acid comprising a terminal olefinic moiety or an optionally substituted peptide comprising a terminal olefinic moiety represented by the structure of Formula (1):

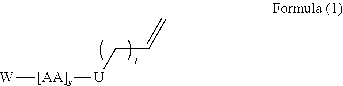

wherein,
AA is any amino acid residue;
U is CH₂, NH, O, or S;
W is hydrogen, a solid support, a functional group, or a protecting group;
t is 0-10; and
s is 1-10.

In one embodiment, the olefin reactant is an optionally substituted amino acid comprising a terminal olefinic moiety or an optionally substituted peptide comprising a terminal olefinic moiety represented by the structure of Formula (2):

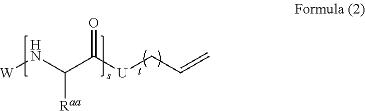

wherein,
s is 1-10;
t is 0-10
U is CH₂, NH, O, or S;
W is hydrogen, a solid support, a functional group, or a protecting group; and
R^{aa} is selected from H, —CH₃, —CH(CH₃)₂, —CH(CH₃)CH₂CH₃, —CH₂(CH(CH₃)₂), —CH₂CH₂SCH₃, —CH₂Ph, —CH₂OH, —CH(OH)CH₃, —CH₂(CO)NH₂, —CH₂CH₂(CO)NH₂, —CH₂SH, —CH₂SeH, —CH₂CH₂CH₂CH₂NH₃⁺, —CH₂(COO)⁻, —CH₂CH₂(COO)⁻,

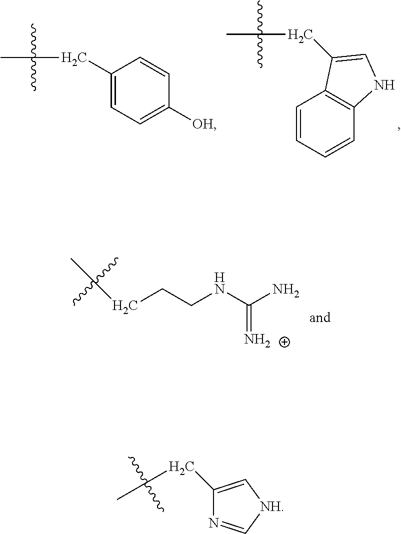 [SMILES: *Cc1ccc(O)cc1 ; *Cc1c[nH]c2ccccc12 ; *CCCNC(N)=[NH2+] ; *Cc1c[nH]cn1]

In one embodiment, the olefin reactant is an optionally substituted amino acid comprising a terminal olefinic moiety or an optionally substituted peptide comprising a terminal olefinic moiety represented by the structure of Formula (3):

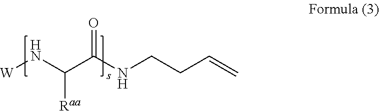

wherein,
s is 1-10;
W is hydrogen, a solid support, a functional group, or a protecting group; and
R^{aa} is selected from H, —CH₃, —CH(CH₃)₂, —CH(CH₃)CH₂CH₃, —CH₂(CH(CH₃)₂), —CH₂CH₂SCH₃, —CH₂Ph, —CH₂OH, —CH(OH)CH₃, —CH₂(CO)NH₂, —CH₂CH₂(CO)NH₂, —CH₂SH, —CH₂SeH, —CH₂CH₂CH₂CH₂NH₃⁺, —CH₂(COO)⁻, —CH₂CH₂(COO)⁻,

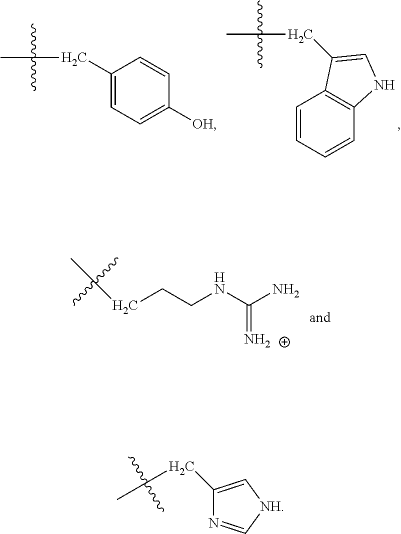 [SMILES: *Cc1ccc(O)cc1 ; *Cc1c[nH]c2ccccc12 ; *CCCNC(N)=[NH2+] ; *Cc1c[nH]cn1]

In the above embodiments, when s is 1, the olefin reactant is an optionally substituted amino acid comprising a terminal olefinic moiety. When s is 2-10, the olefin reactant is an optionally substituted peptide comprising a terminal olefinic moiety.

### Cross Metathesis Product

In one embodiment the cross metathesis product may be represented by the structure of Formula (4):

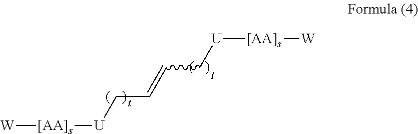

wherein:
AA is independently any amino acid residue;
W is independently hydrogen, a solid support, a functional group, or a protecting group;
U is independently CH₂, NH, O, or S;
t is independently 0-10; and
s is independently 1-10.

In one embodiment, the cross metathesis product may be represented by the structure of Formula (5):

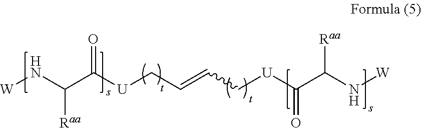

wherein:
s is independently 1-10;
t is independently 0-10
U is independently CH₂, NH, O, or S;
W is independently hydrogen, a solid support, a functional group, or a protecting group; and
R^{aa} is independently selected from H, —CH₃, —CH(CH₃)₂, —CH(CH₃)CH₂CH₃, —CH₂(CH(CH₃)₂), —CH₂CH₂SCH₃, —CH₂Ph, —CH₂OH, —CH(OH)CH₃, —CH₂(CO)NH₂, —CH₂CH₂(CO)NH₂, —CH₂SH, —CH₂SeH, —CH₂CH₂CH₂CH₂NH₃⁺, —CH₂(COO)⁻, —CH₂CH₂(COO)⁻,

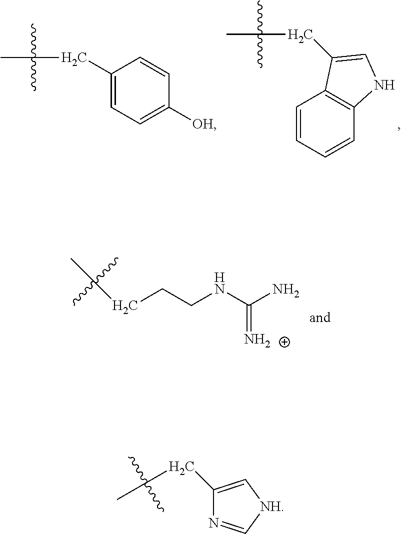 [SMILES: *Cc1ccc(O)cc1 ; *Cc1c[nH]c2ccccc12 ; *CCCNC(N)=[NH2+] ; *Cc1c[nH]cn1]

In one embodiment, the cross metathesis product may be represented by the structure of Formula (6):

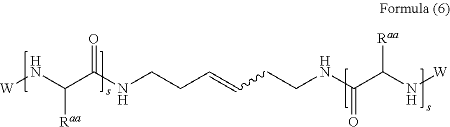

wherein:
s is independently 1-10;
W is independently hydrogen, a solid support, a functional group, or a protecting group; and
R^{aa} is independently selected from H, —CH₃, —CH(CH₃)₂, —CH(CH₃)CH₂CH₃, —CH₂(CH(CH₃)₂), —CH₂CH₂SCH₃, —CH₂Ph, —CH₂OH, —CH(OH)CH₃, —CH₂(CO)NH₂, —CH₂CH₂(CO)NH₂, —CH₂SH, —CH₂SeH, —CH₂CH₂CH₂CH₂NH₃⁺, —CH₂(COO)⁻, —CH₂CH₂(COO)⁻,

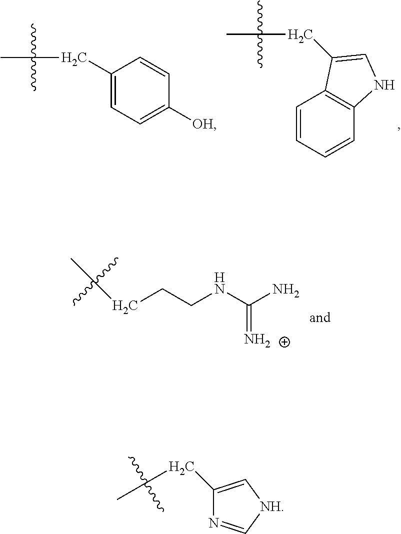 [SMILES: *Cc1ccc(O)cc1 ; *Cc1c[nH]c2ccccc12 ; *CCCNC(N)=[NH2+] ; *Cc1c[nH]cn1]

In some embodiments, the invention provides a method that produces a compound (i.e., a product, olefin product; e.g., cross metathesis product) having a carbon-carbon double bond (e.g., a cross metathesis product internal olefin) in a Z:E ratio greater than about 1:1, greater than about 2:1, greater than about 3:1, greater than about 4:1, greater than about 5:1, greater than about 6:1, greater than about 7:1, greater than about 8:1, greater than about 9:1, greater than about 95:5, greater than about 96:4, greater than about 97:3, greater than about 98:2, or in some cases, greater than about 99:1. In some cases, about 100% of the carbon-carbon double bond produced in the metathesis reaction may have a Z configuration. The Z or cis selectivity may also be expresses as a percentage of product formed (e.g., cross metathesis product). In some cases, the product (e.g., cross metathesis product) may be greater than about 50% Z, greater than about 60% Z, greater than about 70% Z, greater than about 80% Z, greater than about 90% Z, greater than about 95% Z, greater than about 96% Z, greater than about 97% Z, greater than about 98% Z, greater than about 99% Z, or in some cases greater than about 99.5% Z.

### Diolefin Reactant

In one embodiment, the diolefin reactant is an optionally substituted peptide comprising two terminal olefinic moieties.

In another embodiment, the diolefin reactant is an optionally substituted peptide comprising two terminal olefinic moieties represented by the structure of Formula (7):

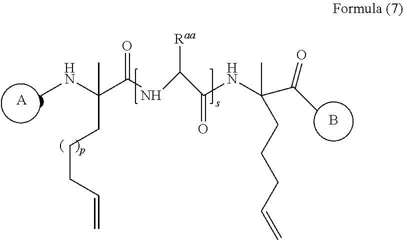

wherein:
s is 1-10;
p is 1-4;
   A is hydrogen, a functional group, a protecting group, an optionally substituted amino acid residue, an optionally substituted peptide residue, a solid support, or any combination thereof;
   B is hydrogen, a functional group, a protecting group, an optionally substituted amino acid residue, an optionally substituted peptide residue, a solid support, or any combination thereof; and
   R^{aa} is selected from H, —CH₃, —CH(CH₃)₂, —CH(CH₃)CH₂CH₃, —CH₂(CH(CH₃)₂), —CH₂CH₂SCH₃, —CH₂Ph, —CH₂OH, —CH(OH)CH₃, —CH₂(CO)NH₂, —CH₂CH₂(CO)NH₂, —CH₂SH, —CH₂SeH, —CH₂CH₂CH₂CH₂NH₃⁺, —CH₂(COO)⁻, —CH₂CH₂(COO)⁻,

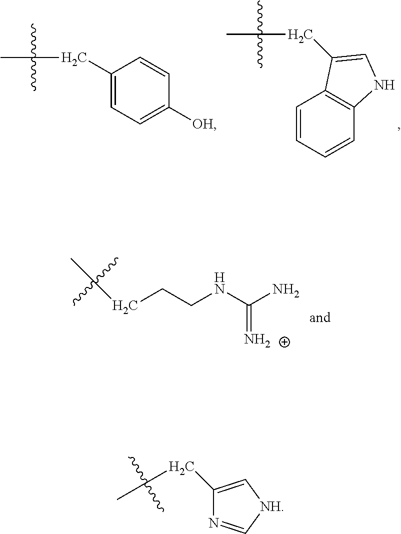 [SMILES: *Cc1ccc(O)cc1 ; *Cc1c[nH]c2ccccc12 ; *CCCNC(N)=[NH2+] ; *Cc1c[nH]cn1]

In another embodiment, the diolefin reactant is an optionally substituted peptide comprising two terminal olefinic moieties represented by the structure of Formula (8):

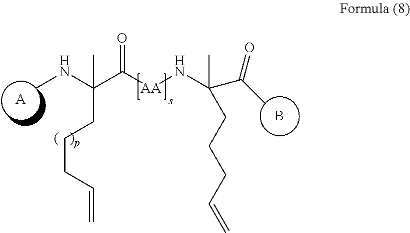

wherein,
AA is any amino acid residue;
A is hydrogen, a functional group, a protecting group, an optionally substituted amino acid residue, an optionally substituted peptide residue, a solid support, or any combination thereof;
B is hydrogen, a functional group, a protecting group, an optionally substituted amino acid residue, an optionally substituted peptide residue, a solid support, or any combination thereof;
p is 1-4; and
s is 1-10.

In another embodiment, the diolefin reactant is an optionally substituted peptide comprising two terminal olefinic moieties represented by the structure of Formula (8a):

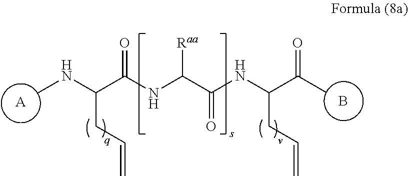

wherein,
A is hydrogen, a functional group, a protecting group, an optionally substituted amino acid residue, an optionally substituted peptide residue, a solid support, or any combination thereof;
B is hydrogen, a functional group, a protecting group, an optionally substituted amino acid residue, an optionally substituted peptide residue, a solid support, or any combination thereof;
q is 1-10;
v is 1-10;
s is 1-10; and
R^{aa} is selected from H, —CH₃, —CH(CH₃)₂, —CH(CH₃)CH₂CH₃, —CH₂(CH(CH₃)₂), —CH₂CH₂SCH₃, —CH₂Ph, —CH₂OH, —CH(OH)CH₃, —CH₂(CO)NH₂, —CH₂CH₂(CO)NH₂, —CH₂SH, —CH₂SeH, —CH₂CH₂CH₂CH₂NH₃⁺, —CH₂(COO)⁻, —CH₂CH₂(COO)⁻,

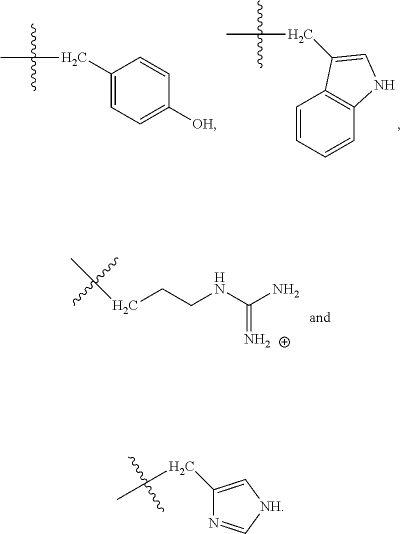 [SMILES: *Cc1ccc(O)cc1 ; *Cc1c[nH]c2ccccc12 ; *CCCNC(N)=[NH2+] ; *Cc1c[nH]cn1]

In another embodiment, the diolefin reactant is an optionally substituted peptide comprising two terminal olefinic moieties represented by the structure of Formula (8b):

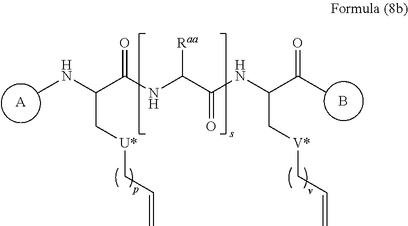

wherein,
A is hydrogen, a functional group, a protecting group, an optionally substituted amino acid residue, an optionally substituted peptide residue, a solid support, or any combination thereof;
B is hydrogen, a functional group, a protecting group, an optionally substituted amino acid residue, an optionally substituted peptide residue, a solid support, or any combination thereof;
p is 0-10;
v is 0-10;
s is 1-10;
U* is nil, CH₂, S, —C₆H₅O—, or O;
V* is nil, CH₂, S, —C₆H₅O—, or O; and
R^{aa} is selected from H, —CH₃, —CH(CH₃)₂, —CH(CH₃)CH₂CH₃, —CH₂(CH(CH₃)₂), —CH₂CH₂SCH₃, —CH₂Ph, —CH₂OH, —CH(OH)CH₃, —CH₂(CO)NH₂, —CH₂CH₂(CO)NH₂, —CH₂SH, —CH₂SeH, —CH₂CH₂CH₂CH₂NH₃⁺, —CH₂(COO)⁻, —CH₂CH₂(COO)⁻,

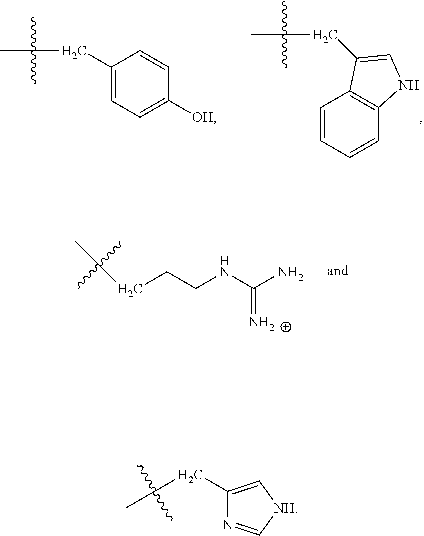 [SMILES: *Cc1ccc(O)cc1 ; *Cc1c[nH]c2ccccc12 ; *CCCNC(N)=[NH2+] ; *Cc1c[nH]cn1]

In another embodiment, the diolefin reactant is an optionally substituted peptide comprising two terminal olefinic moieties represented by the structure of Formula (8c):

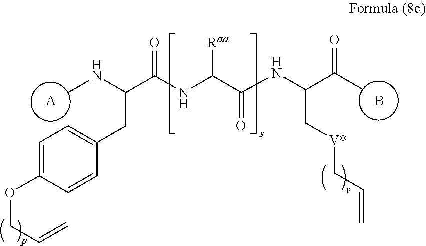

wherein,
A is hydrogen, a functional group, a protecting group, an optionally substituted amino acid residue, an optionally substituted peptide residue, a solid support, or any combination thereof;
B is hydrogen, a functional group, a protecting group, an optionally substituted amino acid residue, an optionally substituted peptide residue, a solid support, or any combination thereof;
p is 0-10;
v is 0-10;
s is 1-10;
V* is nil, CH₂, S, or O; and
R^{aa} is selected from H, —CH₃, —CH(CH₃)₂, —CH(CH₃)CH₂CH₃, —CH₂(CH(CH₃)₂), —CH₂CH₂SCH₃, —CH₂Ph, —CH₂OH, —CH(OH)CH₃, —CH₂(CO)NH₂, —CH₂CH₂(CO)NH₂, —CH₂SH, —CH₂SeH, —CH₂CH₂CH₂CH₂NH₃⁺, —CH₂(COO)⁻, —CH₂CH₂(COO)⁻,

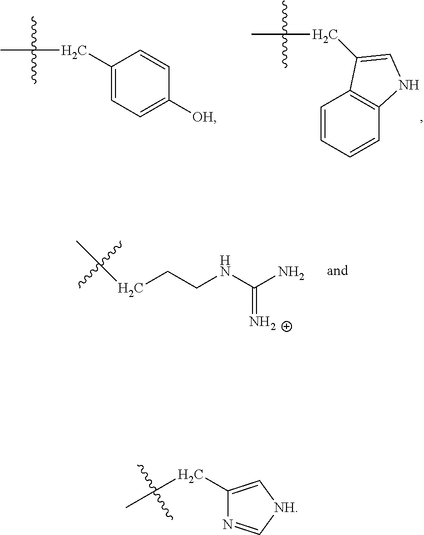 [SMILES: *Cc1ccc(O)cc1 ; *Cc1c[nH]c2ccccc12 ; *CCCNC(N)=[NH2+] ; *Cc1c[nH]cn1]

### Ring-Closing Metathesis Product

In one embodiment, the ring-closing metathesis product is represented by the structure of Formula (9):

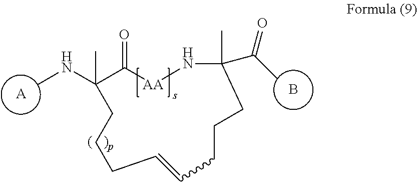

wherein,
AA is any amino acid residue;
A is hydrogen, a functional group, a protecting group, an optionally substituted amino acid residue, an optionally substituted peptide residue, a solid support, or any combination thereof;
B is hydrogen, a functional group, a protecting group, an optionally substituted amino acid residue, an optionally substituted peptide residue, a solid support, or any combination thereof;
p is 1-4; and
s is 1-10.

In another embodiment, the ring closing metathesis product is represented by the structure of Formula (10):

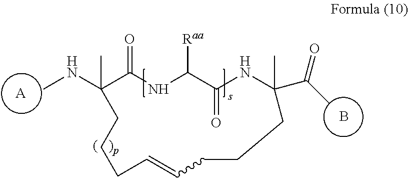

wherein:
R^{aa} is selected from H, —CH₃, —CH(CH₃)₂, —CH(CH₃)CH₂CH₃, —CH₂(CH(CH₃)₂), CH₂CH₂SCH₃, —CH₂Ph, —CH₂OH, —CH(OH)CH₃, —CH₂(CO)NH₂, —CH₂CH₂(CO)NH₂, —CH₂SH, —CH₂SeH, —CH₂CH₂CH₂CH₂NH₃⁺, —CH₂(COO)⁻, —CH₂CH₂(COO)⁻,

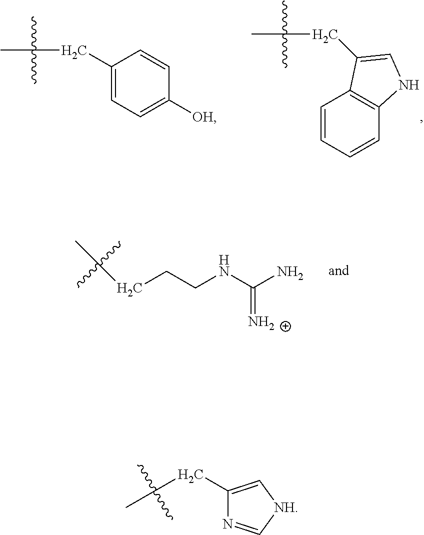 [SMILES: *Cc1ccc(O)cc1 ; *Cc1c[nH]c2ccccc12 ; *CCCNC(N)=[NH2+] ; *Cc1c[nH]cn1]

A is hydrogen, a functional group, a protecting group, an optionally substituted amino acid residue, an optionally substituted peptide residue, a solid support, or any combination thereof;
B is hydrogen, a functional group, a protecting group, an optionally substituted amino acid residue, an optionally substituted peptide residue, a solid support e, or any combination thereof;
p is 1-4; and
s is 1-10.

In another embodiment, the ring closing metathesis product is represented by the structure of Formula (10a):

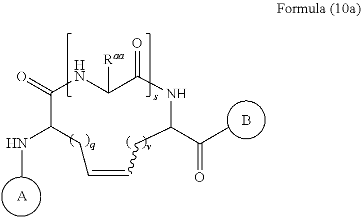

wherein,
A is hydrogen, a functional group, a protecting group, an optionally substituted amino acid residue, an optionally substituted peptide residue, a solid support, or any combination thereof;
B is hydrogen, a functional group, a protecting group, an optionally substituted amino acid residue, an optionally substituted peptide residue, a solid support, or any combination thereof;
q is 1-10;
v is 1-10;
s is 1-10; and
R^{aa} is selected from H, —CH₃, —CH(CH₃)₂, —CH(CH₃)CH₂CH₃, —CH₂(CH(CH₃)₂), —CH₂CH₂SCH₃, —CH₂Ph, —CH₂OH, —CH(OH)CH₃, —CH₂(CO)NH₂, —CH₂CH₂(CO)NH₂, —CH₂SH, —CH₂SeH, —CH₂CH₂CH₂CH₂NH₃⁺, —CH₂(COO)⁻, —CH₂CH₂(COO)⁻,

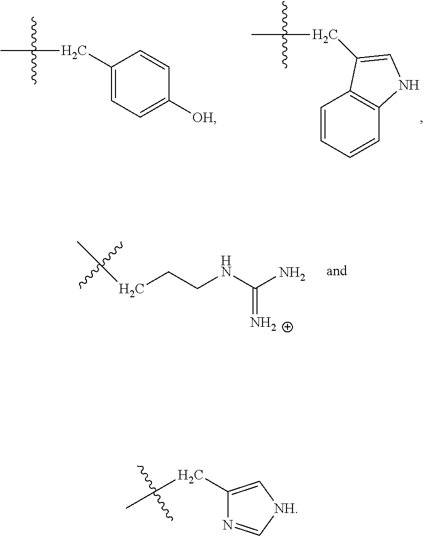 [SMILES: *Cc1ccc(O)cc1 ; *Cc1c[nH]c2ccccc12 ; *CCCNC(N)=[NH2+] ; *Cc1c[nH]cn1]

In another embodiment, the ring-closing metathesis product may be represented by the structure of Formula (10b):

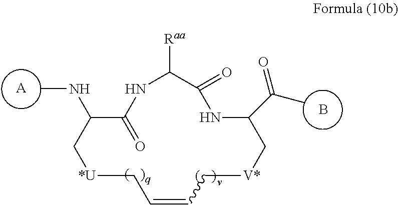

wherein,
A is hydrogen, a functional group, a protecting group, an optionally substituted amino acid residue, an optionally substituted peptide residue, a solid support, or any combination thereof;
B is hydrogen, a functional group, a protecting group, an optionally substituted amino acid residue, an optionally substituted peptide residue, a solid support, or any combination thereof;
q is 0-10;
v is 0-10;
s is 1-10;
U* is nil, CH₂, S, —C₆H₅O—, or O;
V* is nil, CH₂, S, —C₆H₅O—, or O; and

R^{aa} is selected from H, —CH₃, —CH(CH₃)₂, —CH(CH₃)CH₂CH₃, —CH₂(CH(CH₃)₂), —CH₂CH₂SCH₃, —CH₂Ph, —CH₂OH, —CH(OH)CH₃, —CH₂(CO)NH₂, —CH₂CH₂(CO)NH₂, —CH₂SH, —CH₂SeH, —CH₂CH₂CH₂CH₂NH₃⁺, —CH₂(COO)⁻, —CH₂CH₂(COO)⁻,

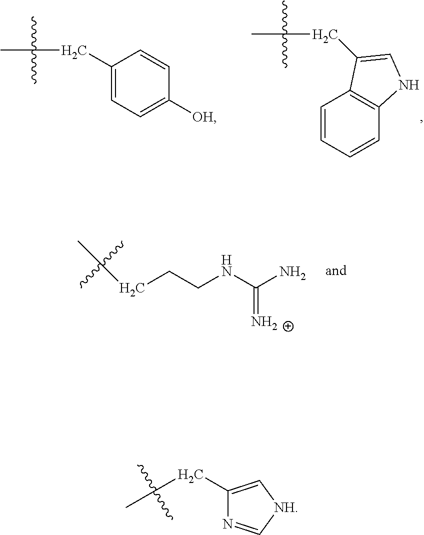 [SMILES: *Cc1ccc(O)cc1 ; *Cc1c[nH]c2ccccc12 ; *CCCNC(N)=[NH2+] ; *Cc1c[nH]cn1]

In some embodiments, and as used in the art, R^{aa} may optionally be represented by the three letter abbreviation for an amino acid, such as Arg, His, Lys, Asp, Glu, Ser, Thr, Asn, Gln, Cys, Sec, Gly, Pro, Ala, Val, Lle, Leu, Met, Phe, Tyr, Trp, where for example in Formula (3):

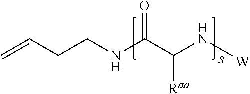

when R^{aa} is Gly then

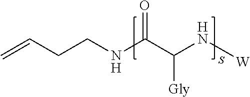

has the same meaning as

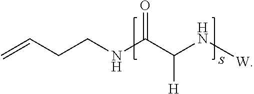

R^{aa} may also be used in a similar manner in other embodiments herein.

In some embodiments, the invention provides a method that produces a compound (i.e., a product, olefin product; e.g., ring-closing metathesis product) having a carbon-carbon double bond (e.g., a ring-closing metathesis product internal olefin) in a Z:E ratio greater than about 1:1, greater than about 2:1, greater than about 3:1, greater than about 4:1, greater than about 5:1, greater than about 6:1, greater than about 7:1, greater than about 8:1, greater than about 9:1, greater than about 95:5, greater than about 96:4, greater than about 97:3, greater than about 98:2, or in some cases, greater than about 99:1. In some cases, about 100% of the carbon-carbon double bond produced in the metathesis reaction may have a Z configuration. The Z or cis selectivity may also be expresses as a percentage of product formed (e.g., ring-closing metathesis product). In some cases, the product (e.g., ring-closing metathesis product) may be greater than about 50% Z, greater than about 60% Z, greater than about 70% Z, greater than about 80% Z, greater than about 90% Z, greater than about 95% Z, greater than about 96% Z, greater than about 97% Z, greater than about 98% Z, greater than about 99% Z, or in some cases greater than about 99.5% Z.

### Assessing the Amino Acid Substrate Scope by Z-Selective Homodimerization

The utility of Z-selective ruthenium catalysts was expanded by examining the influence of amino acids on the selectivity and activity of catalysts Ru-1 and Ru-2. Catalysts bearing N-adamantyl substituents and bidentate nitrato ligands were found to be critical for achieving high Z-selectivity, (see Rosebrugh, L. E.; Herbert, M. B.; Marx, V. M.; Keitz, B. K.; Grubbs, R. H. J. Am. Chem. Soc. 2013, 135, 1276) and it was determined whether such catalysts could be applied to substrates bearing multiple functionalities and with varying steric profiles. In order to benchmark the reactivity of catalysts Ru-1 and Ru-2, homodimerization of protected amino acids modified with homoallyl functionality (Table 1), was investigated. In particular homoallyl-modified amino acids were used to benchmark the intrinsic reactivity of amino acids in homodimerization using cyclometalated ruthenium catalysts. This approach was favored over the analogous allyl-modified amino acids to reduce the chance of competing olefin isomerization that has been observed with cyclometalated ruthenium catalysts. Initial experiments focused on the use of alanine 3 as it was anticipated that amino acids bearing unhindered and aliphatic side chains would provide an ideal platform for comparative experiments. The experiments began with a catalyst loading of 2.5 mol % in THF at 40° C. and this afforded the homodimerization product 4 in 53% yield after 4 hours using catalyst Ru-1 and 58% yield in the presence of catalyst Ru-2 (entry 1). The Z-selectivity remained high (˜90%) throughout the course of the reaction. A reaction time of 4 hours was deemed optimal under the reported conditions. Extended reaction times led to minor amounts of Z-degradation.

TABLE 1
Optimization of homodimerization for homoallyl modified alanine 3

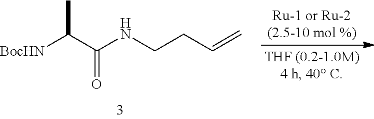 [SMILES: C=CCCNC(=O)[C@H](C)NC(=O)OC(C)(C)C]

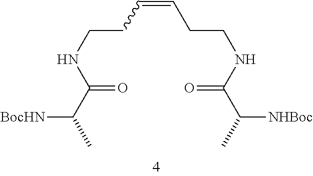

| | | | Yield (%)^{a} | | Z-selectivity (%)^{b} | |
|---|---|---|---|---|---|---|
| Entry | Cat. (mol %) | Conc. (M) | Ru-1 | Ru-2 | Ru-1 | Ru-2 |
| 1 | 2.5 | 0.4 | 53 | 58 | 89 | 93 |
| 2 | 5.0 | 0.2 | 61 | 63 | 86 | 92 |
| 3 | 5.0 | 0.4 | 62 | 63 | 91 | 90 |
| 4 | 5.0 | 1.0 | 60 | 72 | 91 | 91 |
| 5 | 7.5 | 0.2 | 68 | 71 | 90 | 94 |
| 6 | 7.5 | 0.4 | 74 | 76 | 91 | 94 |
| 7 | 7.5 | 1.0 | 72 | 72 | 90 | 91 |
| 8 | 10 | 0.4 | 72 | 70 | 89 | 93 |
| 9 | 10 | 1.0 | 71 | 72 | 90 | 92 |

^{a}Isolated yields
^{b}Determined by ¹H or ¹³C NMR spectroscopy

An increase in catalyst loading was then examined at varying concentrations. Catalyst loadings of 5 mol % afforded product 4 in 65% yield with 92% Z-selectivity in the presence of catalyst Ru-2 (entry 2). Increasing the concentration led to modest improvements in yields while maintaining high Z-selectivity (entries 3, 4). Ultimately, it was found that using a catalyst loading of 7.5 mol % afforded product in 76% yield with 94% Z-selectivity (entry 6) and these conditions proved to be optimal under the various reaction conditions explored. The solvent dependence on the activity of catalysts Ru-1 and Ru-2 in homodimerization (Table 2) was examined next. Several polar and nonpolar solvents were investigated reflecting those most often used in peptide synthesis. Coordinating solvents (e.g., MeCN) were less active in promoting Z-selective metathesis as compared to noncoordinating solvents (e.g., DCE) (entries 2 and 3). Polar solvents including DMF, DMSO, and NMP were tolerated by catalysts Ru-1 and Ru-2 affording product 4 in yields ranging from 55-67% and 90% Z-selectivity (entries 4-6).

TABLE 2
Solvent effects on homodimerization of homoallylmodified alanine 3

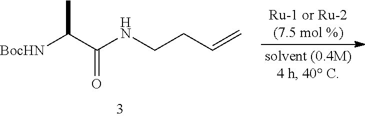 [SMILES: C=CCCNC(=O)[C@H](C)NC(=O)OC(C)(C)C]

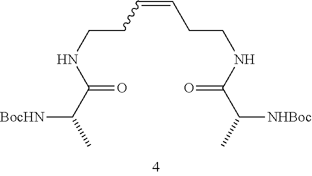

| | | | | Z-selectivity | |
|---|---|---|---|---|---|
| | | Yield (%)^{a} | | (%)^{b} | |
| Entry | Solvent | Ru-1 | Ru-2 | Ru-1 | Ru-2 |
| 1 | THF | 74 | 76 | 91 | 94 |
| 2 | MeCN | 51 | 49 | 88 | 91 |
| 3 | DCE | 72 | 72 | 88 | 92 |
| 4 | DMF | 55 | 59 | 84 | 87 |
| 5 | DMSO | 57 | 55 | 90 | 90 |
| 6 | NMP | 67 | 65 | 87 | 87 |
| 7 | MeOH | 65 | 70 | 85 | 88 |
| 8 | EtOH | 68 | 64 | 88 | 90 |
| 9 | H₂O/tBuOH (1:1) | 64 | 70 | 90 | 92 |
| 10 | MeNO₂ | <10% | <10% | n.d. | n.d. |

^{a}Isolated yields
^{b}Determined by ¹H or ¹³C NMR spectroscopy

Protic solvents including MeOH, EtOH, and aqueous tert-butanol mixtures yielded highly enriched Z-olefin products (entries 7-9). Prolonged heating in the presence of protic solvents can lead to decomposition of catalysts Ru-1 and Ru-2 with concomitant Z-degradation. Other polar solvents including MeNO₂ resulted in low conversions (entry 10), presumably by activating decomposition pathways of the cyclometalated ruthenium catalysts, (see Herbert, M. B.; Lan, Y.; Keitz, B. K.; Liu, P.; Endo, K.; Day, M. W.; Houk, K.; Grubbs, R. H. J. Am. Chem. Soc. 2012, 134, 7861). Across the variety of reaction conditions explored, the Z-selectivity remained consistently high.

To probe further the activity of catalysts Ru-1 and Ru-2, the homodimerization of other canonical amino acids (Table 3) was investigated.

TABLE 3
Homodimerization of canonical amino acids for
investigating side chain influence on catalytic activity

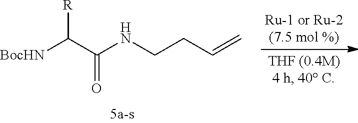

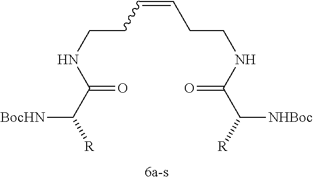

| | | | Yield (%)^{a} | | Z-selectivity (%)^{b} | |
|---|---|---|---|---|---|---|
| Entry | Amino Acid | Product | Ru-1 | Ru-2 | Ru-1 | Ru-2 |
| 1 | Valine (5a) | 6a | 74 | 71 | 90 | 94 |
| 2 | Isoleucine (5b) | 6b | 68 | 72 | 88 | 92 |
| 3 | Leucine (5c) | 6c | 70 | 71 | 88 | 91 |
| 4 | Phenylalanine (5d) | 6d | 73 | 75 | 89 | 93 |
| 5 | Glycine (5e) | 6e | <10 | <10 | n.d. | n.d. |
| 6 | Proline (5f) | 6f | <10 | <5 | n.d | n.d. |
| 7 | Tryptophan (5g) | 6g | 66 | 64 | 85 | 90 |
| 8 | Histidine (5h) | 6h | <5 | <5 | n.d | n.d. |
| 9 | Serine (5i) | 6i | 72 | 70 | 84 | 90 |
| 10 | Threonine (5j) | 6j | 73 | 70 | 88 | 92 |
| 11 | Tyrosine (5k) | 6k | 64 | 68 | 87 | 90 |
| 12 | Methionine (5l) | 6l | <5 | <10 | n.d | n.d |
| 13 | Cysteine (5m)^{c} | 6m | 55 | 53 | 87 | 92 |
| 14 | Aspartic Acid (5n)^{d} | 6n | 61 | 60 | 87 | 90 |
| 15 | Glutamic Acid (5o)^{d} | 6o | 74 | 71 | 88 | 91 |
| 16 | Asparagine (5p)^{c} | 6p | 70 | 71 | 88 | 91 |
| 17 | Glutamine (5q)^{c} | 6q | 74 | 74 | 88 | 91 |
| 18 | Lysine (5r)^{e} | 6r | 78 | 81 | 81 | 89 |
| 19 | Arginine (5s)^{f} | 6s | 34 | 33 | 81 | 89 |

^{a}Isolated yields
^{b}Determined by ¹H or ¹³C NMR spectroscopy
^{c}side chain protected with a trityl group
^{d}side chain protected as the t-butyl ester
^{e}side chain protected as the t-butyl carbamate
^{f}side chain protected with Pbf

Boc-protected amino acids bearing aliphatic or aromatic side chains were active in Z-selective metathesis (entries 1-4). Branched side chains from amino acids including valine (5a), isoleucine (5b), and leucine (5c), afforded products 6a-c in yields approaching 74% with 94% Z-selectivity (entries 1-3). Aromatic side chains from amino acids including phenylalanine 5d afforded product 6d in 75% yield and 93% Z-selectivity in the presence of catalyst Ru-2 (entry 4). Exceptions include glycine (chelation could account for the inactivity of glycine toward homodimerization) and proline. Amino acids bearing heterocycles had variable activity. Homodimerization of tryptophan afforded product 6g in 64% yield with 90% Z-selectivity (entry 7). In contrast, histidine was deemed incompatible with catalysts Ru-1 and Ru-2 (entry 8), protecting the side chain of histidine did not improve the yield of homodimerization. Unprotected alcohols from the side chains of serine 5i or threonine 5j were tolerated by catalysts Ru-1 and Ru-2 but required protection in order to reach acceptable conversions providing products 6i and 6j in 72% and 73% yield, respectively (entries 9, 10). Homodimerization of tyrosine afforded product 6k in 64% yield and 87% Z-selectivity in the presence of catalyst Ru-1 and 68% yield and 90% Z-selectivity using catalyst 2 (entry 11). In contrast to alcohols, side chains bearing thiols (i.e., cysteine) or thioethers (i.e., methionine 5l) generally led to catalyst deactivation (entry 12). These results are consistent with observations that sulfur-containing substrates can have deleterious effects on catalyst activity. Protecting the side chain of cysteine could overcome catalysts inactivity and afford product 6m in 55% yield and 87% Z-selectivity (entry 13). Polar side chains bearing carboxylate (5 n, o), carboxamide (5 p, q) or amine (5r) functionality required protection prior to homodimerization. In these cases, yields from 70-80% could be achieved with high Z-selectivity (entries 14-18). Side chains bearing protected guanidinium functionality (i.e., arginine 5s) were less active in homodimerization affording product 6s in 34% and 33% yield using catalysts Ru-1 and Ru-2, respectively. These findings reveal that the identity of the amino acid side chain can profoundly influence the activity of cyclometalated ruthenium catalysts.

### Assessing Side Chain Influence and Stoichiometry on Cross Metathesis of Amino Acids

Given the success of Z-selective homodimerization of amino acids, the effect of varying the metathesis partner to achieve Z-selective cross metathesis (CM) (Table 4) was evaluated next. For these experiments, cross partners were chosen that showed variable activity in homodimerization. In this way, substrates based upon their propensity for achieving productive CM could be classified, (see Chatterjee, A. K.; Choi, T.-L.; Sanders, D. P.; Grubbs, R. H. J. Am. Chem. Soc. 2003, 125, 11360). CM using homoallyl-modified alanine 3 and the similarly reactive substrate valine 5a as the cross partner was explored. Cross metathesis in the presence of an equimolar ratio of 3 to 5a afforded the desired heterocross product 7 in 41% yield with 90% Z-selectivity (entry 1). Increasing the stoichiometry of 5a relative to 3 led to a modest increase in yield providing product 7 in 48% yield with 91% Z-selectivity (entry 2). Further increase in 5a afforded similar trends, with yields approaching 60% and 90% Z-selectivity (entries 3, 4). In order to demonstrate the versatility of this method, conditions in which 5a was used as the limiting reagent (entries 5-7) was explored. Under these conditions, an incremental increase in the yield of product 7 was observed upon increasing the ratio of 3 to 5a. As in the case of excess 5a, modest improvements in the yield of 7 were achieved with increasing equivalents of 3, demonstrating the statistical nature of CM using similar reactive substrates. Exposing the dimer of substrates 3 or 5a to the reaction conditions did not improve the yield of product 7, nor lead to significant Z-degradation. This supports the observation that secondary metathesis events occur slowly with homoallyl-modified amino acids. Given the modest conversions to product and high Z-selectivity, the products of CM are sparingly consumable using catalysts Ru-1 and Ru-2 ensuring that the Z-selectivity can remain high throughout the course of the reaction.

TABLE 4
Cross metathesis of amino acids 3 and 5a

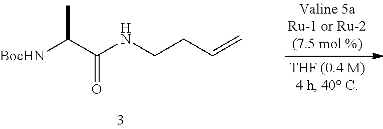

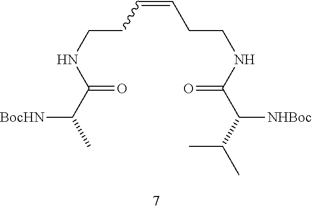 [SMILES: CC(C)[C@@H](NC(=O)OC(C)(C)C)C(=O)NCCC=CCCNC(=O)[C@H](C)NC(=O)OC(C)(C)C]

| | | | Yield (%)^{a} | | Z-selectivity (%)^{b} | |
|---|---|---|---|---|---|---|
| Entry | equiv. 3 | equiv. 5a | Ru-1 | Ru-2 | Ru-1 | Ru-2 |
| 1 | 1 | 1 | 41 | 47 | 90 | 93 |
| 2 | 1 | 2 | 44 | 48 | 86 | 91 |
| 3 | 1 | 4 | 48 | 41 | 91 | 90 |
| 4 | 1 | 6 | 58 | 60 | 88 | 90 |
| 5 | 2 | 1 | 44 | 58 | 90 | 93 |
| 6 | 4 | 1 | 52 | 57 | 91 | 91 |
| 7 | 6 | 1 | 51 | 60 | 87 | 90 |

^{a}Isolated yield
^{b}Determined by ¹H or ¹³C NMR spectroscopy

Next, amino acids of differing reactivity profiles were evaluated whether they could be used for selective CM (Table 5). In choosing the requisite cross partners, substrate 3 and homoallyl-modified arginine 5s were focused on as they both had shown relatively low reactivity in homodimerization. An equimolar ratio of 3 and 5s were used to begin the experiment and this afforded 8 in 46% yield and 93% Z-selectivity (entry 1). Increasing the ratio of 5s to 3 led to slightly diminished yields and Z-selectivity (entries 2-4). Using an excess of arginine 5s can lead to minor amounts of catalyst decomposition and Z-degradation over extended reaction times. By contrast, reversing the order such that 3 was in excess of 5s afforded 8 in yields of 47% and 58% using catalysts Ru-1 and Ru-2, respectively (entry 5). Further increasing the ratio of 3 to 5s could achieve the heterocross product 8 in improved yields and high Z-selectivity (entries 6 and 7). Taken together, these findings reveal that the intrinsic reactivity differences between homoallyl-modified amino acids can be used for productive Z-selective cross metathesis.

TABLE 5
Cross metathesis of amino acid 3 and 5s

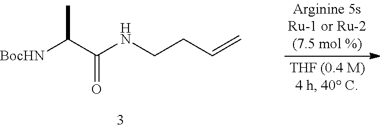

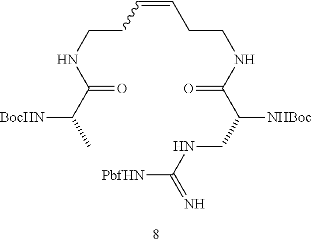

| | | | Yield (%)^{a} | | Z-selectivity (%)^{b} | |
|---|---|---|---|---|---|---|
| Entry | equiv. 3 | equiv. 5s | Ru-1 | Ru-2 | Ru-1 | Ru-2 |
| 1 | 1 | 1 | 46 | 47 | 90 | 93 |
| 2 | 1 | 2 | 43 | 48 | 84 | 90 |
| 3 | 1 | 4 | 38 | 41 | 84 | 91 |
| 4 | 1 | 6 | 34 | 38 | 88 | 88 |
| 5 | 2 | 1 | 47 | 58 | 72 | 91 |
| 6 | 4 | 1 | 58 | 60 | 88 | 92 |
| 7 | 6 | 1 | 62 | 66 | 87 | 90 |

^{a}Isolated yield
^{b}Determined by ¹H or ¹³C NMR spectroscopy

### The Influence of Allylic Heteroatoms on Homodimerization and Cross Metathesis of Non-Canonical Amino Acids

The unique reactivity profile of canonical amino acids in Z-selective homodimerization and CM prompted investigation into a subset of non-natural amino acids that have shown promise in peptide and protein modification using olefin metathesis. Allyl-protected amino acids including serine, cysteine, and selenocysteine have been shown to enhance the rate of metathesis when incorporated into peptides and proteins, (see Lin, Y. A.; Chalker, J. M.; Davis, B. G. J. Am. Chem. Soc. 2010, 132, 16805; Chalker, J. M.; Goncalo, J. L. B.; Davis, B. D. Acc. Chem. Res. 2011, 44, 73). Studies by Davis et al. have ascribed the unique chemical reactivity of such amino acids through a chelation-assisted mechanism whereby precoordination of the heteroatom to ruthenium increases the effective concentration of the alkylidene and alkene without detrimental chelation, (see Lin, Y. A.; Chalker, J. M.; Davis, B. G. J. Am. Chem. Soc. 2010, 132, 16805). Moreover, softer nucleophiles such as sulfur and selenium were found to have an activating effect relative to oxygen for enhancing the rate of CM. These findings are intriguing considering that sulfur can have a deactivating effect on olefin metathesis, (see Ben-Asuly, A. T., E.; Diesendruck, C. E.; Sigalov, M. G., I.; Lemcoff, G. N. Organometallics 2008, 27, 811; McReynolds, M. D.; Dougherty, J. M.; Hanson, P. R. Chem. Rev. 2004, 104, 2239; Schwab, P.; Grubbs, R. H.; Ziller, J. W. J. Am. Chem. Soc. 1996, 118, 100) and the results suggest that sulfur-containing amino acids (e.g., compound 51) lead to catalyst inactivity. Nonetheless, a wealth of information suggests that heteroatoms can modulate metathesis activity, (see Hoveyda, A. H.; Lombardi, P. J.; O'Brien, R. V.; Zhugralin, A. R. J. Am. Chem. Soc. 2009, 131, 8378; Hoye, T. R.; Zhao, H. Org. Lett. 1999, 1, 1123; Nicolaou, K. C.; Leung, G. Y. C.; Dethe, D. H.; Guduru, R.; Sun, Y.-P.; Lim, C. S.; Chen, D. Y. K. J. Am. Chem. Soc. 2008, 130, 10019) however this phenomenon was unexplored using Z-selective cyclometalated catalysts.

To investigate the influence of allylic heteroatoms on the activity of catalysts Ru-1 and Ru-2, a series of allyl-protected amino acids in homodimerization (Table 6) were examined.

TABLE 6
The influence of heteroatoms on homodimerization of non-canonical amino acids

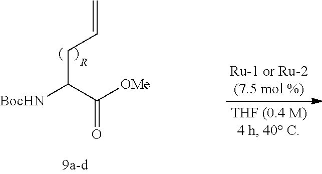

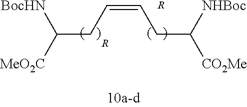

| | | | Yield (%)^{a} | | Z-selectivity (%)^{b} | |
|---|---|---|---|---|---|---|
| Entry | R | Product | Ru-1 | Ru-2 | Ru-1 | Ru-2 |
| 1 | CH₂ (9a) | 10a | 44 | 46 | 88 | 93 |
| 2 | CH₂CH₂ (9b) | 10b | 59 | 58 | 90 | 92 |
| 3 | CH₂OCH₂ (9c) | 10c | 67 | 69 | 92 | 94 |
| 4 | CH₂SCH₂ (9d) | 10d | 74 | 71 | 90 | 90 |

^{a}Isolated yield
^{b}Determined by ¹H or ¹³C NMR spectroscopy

Allylglycine (9a), homoallylglycine (9b), as well as allyl-protected serine (9c) and cysteine (9d) were chosen for these experiments. In this sense, 9a-d would reveal both the role of sterics (i.e., comparison of 9a and 9b) and the effect of chelation by heteroatoms (i.e., comparison of 9b to 9c, d) in facilitating metathesis. To test this, homodimerization of 9a was investigated and its reactivity was compared relative to substrate 9b. Conversion of 9a to the dimerized product 10a was low using catalysts Ru-1 or Ru-2 occurring in 45% yield and 90% Z-selectivity (entry 1). By comparison, dimerization of homoallylglycine 9b afforded product 10b in 59% yield and 90% Z-selectivity (entry 2). This corroborates earlier findings that the steric environment around the alkene can influence the efficiency of Z-selective metathesis, (see Quigley, B. L.; Grubbs, R. H. Chem. Sci. 2014, 5, 501). The effect of heteroatoms in facilitating homodimerization was evaluated next. Allyl-protected serine 9c afforded product 10c in 67% yield with 92% Z-selectivity (entry 3). By comparison, cysteine 9d was more active in homodimerization, leading to 71% yield and 93% Z-selectivity in the presence of catalyst Ru-2 (entry 4). These results suggest that allylic heteroatoms can facilitate Z-selective metathesis in the presence of cyclometalated ruthenium catalysts.

The insights garnered from the homodimerization of substrates 9a-d revealed that the identity of the alkene can influence the activity of catalysts Ru-1 and Ru-2 in Z-selective metathesis. While these experiments provide important insight for assessing the intrinsic reactivity of allyl-modified amino acids with cyclometalated ruthenium catalysts, the general utility of such catalysts were further illuminated by their use in CM. As such, catalysts Ru-1 and Ru-2 in CM using allyl-modified amino acids 9a-d (Table 7) were examined. To assess the relative activity of substrates 9a-d in CM, allyl acetate 11 was chosen as the cross partner as it has been shown previously that 11 is highly active in Z-selective CM, (see Hartung, J.; Grubbs, R. H. J. Am. Chem. Soc. 2013, 135, 10183).

TABLE 7
Cross Metathesis of Allyl-Modified Amino Acids with Allyl Acetate

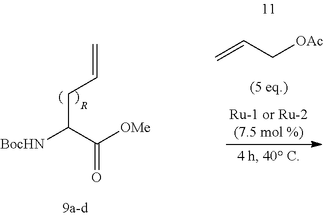

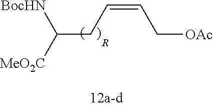

| | | | Yield (%)^{a} | | Z-selectivity (%)^{b} | |
|---|---|---|---|---|---|---|
| Entry | Substrate | Product | Ru-1 | Ru-2 | Ru-1 | Ru-2 |
| 1^{c} | (9a) | (12a) | 40 | 42 | 88 | 90 |
| 2^{d} | | | 38 | 36 | 90 | 92 |
| 3^{e} | | | 31 | 31 | 76 | 84 |
| 4^{f} | | | 30 | 34 | 72 | 83 |
| 5^{c} | (9b) | (12b) | 56 | 55 | 90 | 95 |
| 6^{d} | | | 53 | 51 | 90 | 93 |
| 7^{e} | | | 48 | 50 | 84 | 87 |
| 8^{f} | | | 44 | 46 | 79 | 84 |
| 9^{c} | (9c) | (12c) | 63 | 66 | 88 | 92 |
| 10^{d} | | | 64 | 67 | 87 | 93 |
| 11^{e} | | | 54 | 61 | 67 | 79 |
| 12^{f} | | | 56 | 61 | 63 | 84 |
| 13^{c} | (9d) | (12d) | 62 | 61 | 88 | 90 |
| 14^{d} | | | 63 | 67 | 86 | 92 |
| 15^{e} | | | 55 | 60 | 74 | 88 |
| 16^{f} | | | 58 | 60 | 76 | 82 |

^{a}Isolated yields
^{b}Determined by ¹H or ¹³C NMR spectroscopy
^{c}Reaction conditions: 1 mmol 9a-d in THF
^{d}Reaction conditions: 1 mmol 9a-d in 1:1 tert-butanol:H₂O
^{e}Reaction conditions: 1 mmol 9a-d in 1:1 tert-butanol:H₂O + 2 mM LiCl
^{f} eaction conditions: 1 mmol 9a-d in 1:1 tert-butanol:H₂O + 2 mM MgCl₂

A variety of conditions were explored to test the generality of CM including the use of solvents that are compatible with native peptides and proteins. For the initial experiments, CM between allylglycine 9a and 11 was examined under previously optimized conditions and this afforded the heterocross product 12a in 42% yield and 90% Z-selectivity. CM under aqueous conditions was explored next, including the use of additives shown to enhance the efficiency of CM on peptides and proteins, (see Lin, Y. A.; Chalker, J. M.; Davis, B. G. J. Am. Chem. Soc. 2010, 132, 16805). In the presence of aqueous tert-butanol, the heterocross product 12a was achieved in 38% yield with 90% Z-selectivity (entry 2). Inclusion of salts such as LiCl or MgCl₂ as shown to be beneficial for enhancing methathesis on peptides, (see Roberts, K. S.; Sampson, N. S. J. Org. Chem. 2003, 68, 2020; Whelan, A. N.; Elaridi, J.; Mulder, R. J.; Robinson, A. J.; Jackson, W. R. Can. J. Chem. 2005, 83, 875) afforded product 12a in 31% yield but with diminished Z-selectivity (entries 3, 4). These trends were also observed using homoallylglycine 9b as the cross partner (entries 5-8). To investigate whether amino acids bearing allylic heteroatoms influence the efficiency of Z-selective CM, allylserine 9c and allylcysteine 9d were exposed to similar reaction conditions. Synthesis of the heterocross product was improved, affording 12c in 63% yield with 92% Z-selectivity using catalyst 2 (entry 9). The use of aqueous conditions (entry 10) or inclusion of additives (entries 11 and 12) led to slightly diminished yields and Z-selectivity. By comparison, the use of allylcysteine 9d as the cross partner afforded the desired product 12d in 60% yield with 90% Z-selectivity (entry 13). As observed with substrates 9a-c, a decrease in catalyst activity occurred under aqueous conditions and in the presence of salt additives (entries 14-16). It is believed that salt metathesis could account for the lower activity of catalysts Ru-1 and Ru-2 in the presence of lithium chloride or magnesium chloride. Exchange of a bidentate nitrato ligand to a monodentate chloride ligand has been observed to decrease the catalytic activity of cyclometalated ruthenium complexes. Collectively, these results suggest that the activity of catalysts Ru-1 and Ru-2 is highly dependent on the reaction conditions. In general, the trends observed in CM with 9a-d parallel those observed using non-chelated ruthenium catalysts, (see Lin, Y. A.; Chalker, J. M.; Floyd, N.; Bernardes, C. J. L.; Davis, B. G. J. Am. Chem. Soc. 2008, 130, 9642) however the use of salts as additives appears to have a deactivating effect on the activity of cyclometalated ruthenium catalysts. These results lend support to the importance of the chelating ligand (i.e., bidentate versus mondentate ligand coordination) in the activity and selectivity of catalysts Ru-1 and Ru-2.

### Z-Selective Cross Metathesis on Linear Peptides

Investigation of both canonical and non-canonical amino acids in homodimerization and cross metathesis revealed that the choice of cross partner is critical to the success of ruthenium-catalyzed Z-selective metathesis. In this regard, amino acid side chains bearing aliphatic, aromatic, or protected polar functionality were highly active in metathesis. Those amino acids that generally lead to lower conversion (i.e., arginine) could undergo selective cross metathesis in the presence of a more highly reactive substrate. Moreover, the steric environment around the olefin and allylic heteroatoms were shown to impact the efficiency of Z-selective CM. While these observations apply generally to amino acids that are commonly used in olefin metathesis, it was investigated whether cyclometalated ruthenium catalysts could be used in CM on more complex substrates, including peptides. In choosing the requisite cross partners, peptides known to adopt defined β-sheet secondary structures when tethered via a turn promoting moiety or as part of a macrocycle were taken into consideration, (see Almeida, A. M.; Li, R.; Gellman, S. H. J. Am. Chem. Soc. 2012, 134, 75; Freire, F.; Almeida, A. M.; Fisk, J. D.; Steinkruger, J. D.; Gellman, S. H. Angew. Chem. Int. Ed. 2011, 50, 8735; Woods, R. J.; Brower, J. O.; Castellanos, E.; Hashemzadeh, M.; Khakshoor, O.; Russu, W. A.; Nowick, J. S. J. Am. Chem. Soc. 2007, 129, 2548). Such structures hold promise in applications ranging from supramolecular chemistry (see Cheng, P. N.; Pham, J. D.; Nowick, J. S. J. Am. Chem. Soc. 2013, 135, 5477; Nowick, J. S. Acc. Chem. Res. 2008, 41, 1319) to biology, (see Cheng, P. N.; Liu, C.; Zhao, M.; Eisenberg, D.; Nowick, J. S. Nat. Chem. 2012, 4, 927; Chiti, F.; Dobson, C. M. Annu. Rev. Biochem. 2006, 75, 333; Karran, E.; Mercken, M.; De Strooper, B. Nat. Rev. Drug Discov. 2011, 10, 698) and offer challenging substrates for catalysts Ru-1 and Ru-2.

A wealth of information regarding the use of peptides and small molecules as β-sheet mimics has revealed that both hydrogen bonding and amino acids side chain pairing preferences can be used to dictate the stability of β-sheet formation, (see Almeida, A. M.; Li, R.; Gellman, S. H. J. Am. Chem. Soc. 2012, 134, 75; Fisk, J. D.; Gellman, S. H. J. Am. Chem. Soc. 2001, 123, 343; Fooks, H. M.; Martin, A. C.; Woolfson, D. N.; Sessions, R. B.; Hutchinson, E. G. J. Mol. Biol. 2006, 356, 32). To this end, peptides 13 and 14 were synthesized that represent typical sequences found in parallel β-sheets and whose structure is known to rely on the sequence of amino acids (Scheme 3), (see Almeida, A. M.; Li, R.; Gellman, S. H. J. Am. Chem. Soc. 2012, 134, 75).

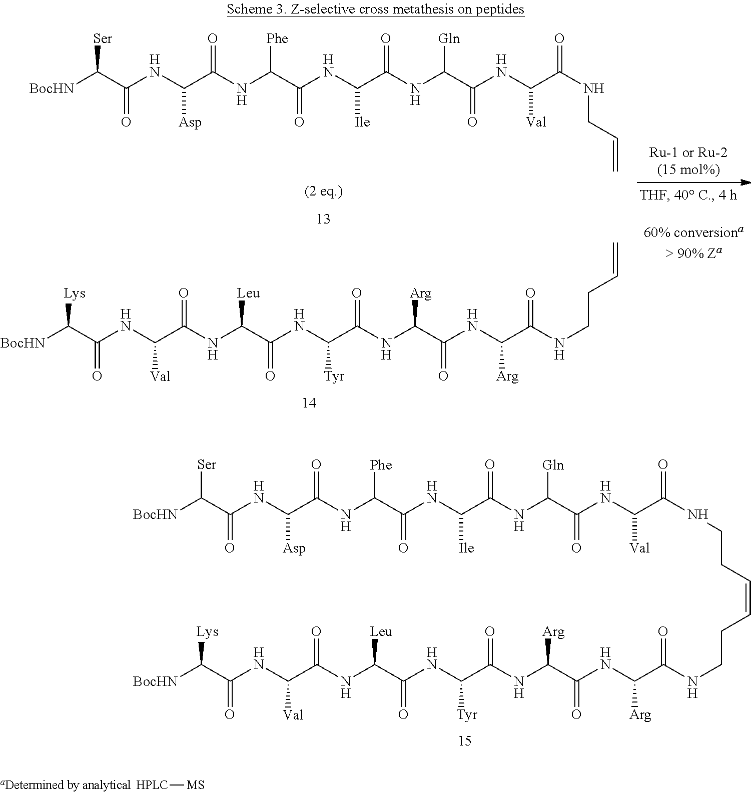

For the experiments, an excess of peptide 13 was used as is has been shown that less reactive amino acids, including arginine, could be used for selective cross metathesis in the presence of more highly active amino acids. Under these conditions, conversion reached 60% to the desired peptide 15 with greater than 90% Z-selectivity. The remaining mass balance in the Z-selective CM of peptides 13 and 14 can be attributed to unreacted starting material and by-products resulting from homodimerization.

Attempts at improving the yield of the heterocross product by conducting CM in solvents that promote hydrogen-bonding and thereby preorganize the peptides to facilitate metathesis, (see Clark, T. D.; Ghadiri, M. R. J. Am. Chem. Soc. 1995, 117, 12364; Yang, X.; Gong, B. Angew. Chem. Int. Ed. 2005, 44, 1352; Zeng, J.; Wang, W.; Deng, P.; Feng, W.; Zhou, J.; Yang, Y.; Yuan, L.; Yamato, K.; Gong, B. Org. Lett. 2011, 13, 3798) led to similar conversions. Performing cross metathesis in the presence of solvents that can influence interstrand hydrogen bonding including DMSO, DCE, or aqueous tert-butanol mixtures showed no appreciable increase in the yield of the heterocross product. These results attest to the functional group tolerability of cyclometalated ruthenium catalysts and point to further strategies aimed at accessing complex olefinic substrates bearing multiple functionalities.

### Z-Selective Ring-Closing Metathesis on α-Helical Peptides

The results regarding the activity of cyclometalated ruthenium catalysts in cross metathesis of amino acids and peptides revealed that the kinetic selectivity of catalysts Ru-1 and Ru-2 can be used to synthesize peptides highly enriched in Z-olefins. While these experiments provide a framework for promoting metathesis through judicious choice of the amino acid sequence, it was observed that conversions in CM were optimal at relatively high concentrations (˜0.3-0.4 M) which, for many substrates, may be a limitation. A more general strategy was sought, such that a variety of reaction conditions could be used to promote Z-selective metathesis on a diverse collection of peptides. To this end, ring-closing metathesis (RCM) of olefinic amino acids for the synthesis of ‘stapled’ peptides was investigated. Such peptides hold promise as novel therapeutics by virtue of their enhanced α-helicity, (see Schafmeister, C. E.; Po, J.; Verdine, G. L. J. Am. Chem. Soc. 2000, 122, 5891; Guo, Z.; Mohanty, U.; Noehre, J.; Sawyer, T. K.; Sherman, W.; Krilov, G. Chem. Biol. Drug Des. 2010, 75, 348) proteolytic stability, (see Walensky, L. D.; Kung, A. L.; Escher, I.; Malia, T. J.; Barbuto, S.; Wright, R. D.; Wagner, G.; Verdine, G. L.; Korsmeyer, S. J. Science 2004, 305, 1466; Bird, G. H.; Madani, N.; Perry, A. F.; Princiotto, A. M.; Supko, J. G.; He, X.; Gavathiotis, E.; Sodroski, J. G.; Walensky, L. D. Proc. Natl. Acad. Sci. U.S.A 2010, 107, 14093; Bernal, F.; Tyler, A. F. K., S. J. Walensky, L. D. Verdine, G. L. J. Am. Chem. Soc. 2007, 129, 2456; Bird, G. H.; Bernal, F.; Pitter, K.; Walensky, L. D. Methods Enzymol. 2008, 446, 369; Chapuis, H.; Slaninova, J.; Bednarova, L.; Monincova, L.; Budesinsky, M.; Cerovsky, V. Amino acids 2012, 43, 2047; Green, B. R.; Klein, B. D.; Lee, H. K.; Smith, M. D.; White, S. H.; Bulaj, G. Bioorg. Med. Chem. 2013, 21, 303; Sviridov, D. O.; Ikpot, I. Z.; Stonik, J.; Drake, S. K.; Amar, M.; Osei-Hwedieh, D. O.; Piszczek, G.; Turner, S.; Remaley, A. T. Biochem. Biophys. Res. Commun. 2011, 410, 446), and ability to target intracellular proteins involved in cancer, (see Verdine, G. L.; Hilinski, G. J. Methods Enzymol. 2012, 503, 3; Bernal, F.; Wade, M.; Godes, M.; Davis, T. N.; Whitehead, D. G.; Kung, A. L.; Wahl, G. M.; Walensky, L. D. Cancer cell 2010, 18, 411; Chang, Y. S.; Gravesb, B.; Guerlavaisa, V.; Tovarb, C.; Packmanb, K.; Tob, K.-H.; Olsona, K. A.; Kesavana, K.; Gangurdea, P.; Mukherjeea, A.; Bakera, T.; Darlaka, K.; Elkina, C.; Filipovich, Z.; Qureshib, F. Z.; Caia, H.; Berry, P.; Feyfanta, E.; Shia, X. E.; Horsticka, J.; Annisa, D. A.; Manninga, A. M.; Fotouhib, N.; Nasha, H.; Vassilev, L. T.; Sawyer, T. K. Proc. Natl. Acad. Sci. U.S.A 2013, 110, e3445; Bernal, F.; Tyler, A. F. K., S. J. Walensky, L. D. Verdine, G. L. J. Am. Chem. Soc. 2007, 129, 2456), infectious diseases, (see Long, Y. Q.; Huang, S. X.; Zawahir, Z.; Xu, Z. L.; Li, H.; Sanchez, T. W.; Zhi, Y.; DeHouwer, S.; Christ, F.; Debyser, Z.; Neamati, N. J. Med. Chem. 2013, 56, 5601; Zhang, H.; Curreli, F.; Waheed, A. A.; Mercredi, D. Y.; Mehta, M.; Bhargava, P.; Scacalossi, D.; Tong, X.; Lee, S.; Cooper, A.; Summers, M. F.; Freed, E. O.; Debnath, A. K. Retrovirology 2013, 10, 136; Zhang, H.; Zhao, Q.; Bhattacharya, S.; Waheed, A. A.; Tong, X.; Hong, A.; Heck, S.; Curreli, F.; Goger, M.; Cowburn, D.; Freed, E. O.; Debnath, A. K. J. Mol. Biol. 2008, 378, 565) and metabolism, (see Bird, G. H.; Gavathiotis, E.; Labelle, J. L.; Katz, S. G.; Walensky, L. D. ACS Chem. Biol. 2014, 9, 831). As such, the invention disclosed herein broadens the available catalysts used to synthesize this important class of peptides under conditions that would be amenable to comprehensive screening of catalyst activity in the presence of varying peptide sequences.

Traditional methods for the synthesis of stapled peptides via RCM have relied on the use of O-allyl serine, (see Blackwell, H. B.; Grubbs, R. H. Angew. Chem. Int. Ed. 1998, 37, 3281; Blackwell, H. B.; Sadowsky, J. D.; Howard, R. J.; Sampson, N. S.; Chao, J. A.; Steinmetz, W. E.; O'Leary, D. J.; Grubbs, R. H. J. Org. Chem. 2001, 66, 5291; Boal, A. K.; Guryanov, I.; Moretto, A.; Crisma, M.; Lanni, E. L.; Toniolo, C.; Grubbs, R. H.; O'Leary, D. J. J. Am. Chem. Soc. 2007, 129, 6986) or Cα-tetrasubstituted amino acids, (see Schafmeister, C. E.; Po, J.; Verdine, G. L. J. Am. Chem. Soc. 2000, 122, 5891; Kim, Y. W.; Grossmann, T. N.; Verdine, G. L. Nat. Protoc. 2011, 6, 761; Toniolo, C.; Benedetti, E. Trends Biochem. Sci. 1991, 16, 350; Bird, G. H.; Crannell, W. C.; Walensky, L. D. Curr. Protoc. Chem. Biol. 2011, 3, 99) to install macrocyclic crosslinks into synthetic peptides. Most strategies incorporate non-natural amino acids at positions spanning across one (i, i+4) or two (i, i+7) turns of a helix that serves to preorganize the reactive side chains on the same helical face. A wealth of knowledge derived from computational, (see Kutchukian, P. S.; Yang, J. S.; Verdine, G. L.; Shakhnovich, E. I. J. Am. Chem. Soc. 2009, 131, 4622; Vanhee, P.; van der Sloot, A. M.; Verschueren, E.; Serrano, L.; Rousseau, F.; Schymkowitz, J. Trends Biotechnol. 2011, 29, 231) and experimental, (see Chang, Y. S.; Gravesb, B.; Guerlavaisa, V.; Tovarb, C.; Packmanb, K.; Tob, K.-H.; Olsona, K. A.; Kesavana, K.; Gangurdea, P.; Mukherjeea, A.; Bakera, T.; Darlaka, K.; Elkina, C.; Filipovich, Z.; Qureshib, F. Z.; Caia, H.; Berry, P.; Feyfanta, E.; Shia, X. E.; Horsticka, J.; Annisa, D. A.; Manninga, A. M.; Fotouhib, N.; Nasha, H.; Vassilev, L. T.; Sawyer, T. K. Proc. Natl. Acad. Sci. U.S.A 2013, 110, e3445; Boal, A. K.; Guryanov, I.; Moretto, A.; Crisma, M.; Lanni, E. L.; Toniolo, C.; Grubbs, R. H.; O'Leary, D. J. J. Am. Chem. Soc. 2007, 129, 6986; Kim, Y.-W.; Kutchukian, P. S.; Verdine, G. L. Org. Lett. 2010, 12, 3046; Kim, Y. W.; Verdine, G. L. Bioorg. Med. Chem. Lett. 2009, 19, 2533) approaches have illuminated the minimal constraints necessary for achieving RCM on peptides using first- and second-generation or Grubbs-Hoveyda ruthenium catalysts, (see Bergman, Y. E.; Del Borgo, M. P.; Gopalan, R. D.; Jalal, S.; Unabia, S. E.; Ciampini, M.; Clayton, D. J.; Fletcher, J. M.; Mulder, R. J.; Wilce, J. A.; Aguilar, M.-I.; Perlmutter, P. Org. Lett. 2009, 11, 4438; Shim, S. Y.; Kim, Y. W.; Verdine, G. L. Chem. Biol. Drug Des. 2013, 82, 635). An unmet challenge in the synthesis of stapled peptides has been the ability to control olefin geometry in the product as the use of non-cyclometalated ruthenium catalysts typically give rise to both E and Z isomers that are often inseparable. This imposes challenges for examining the role of olefin geometry on the stability and biological activity of stapled peptides which, to date, has not been thoroughly explored, (see Chapuis, H.; Slaninova, J.; Bednarova, L.; Monincova, L.; Budesinsky, M.; Cerovsky, V. Amino acids 2012, 43, 2047; Cianni, A. D.; Carotenuto, A.; Brancaccio, D.; Novellino, E.; Reubi, J. C.; Beetschen, K.; Papini, A. M.; Ginanneschi, M. J. Med. Chem. 2010, 53, 6188; Stymiest, J. L.; Mitchell, B. F.; Wong, S.; Vederas, J. C. J. Org. Chem. 2005, 70, 7799; van Lierop, B. J.; Robinson, S. D.; Kompella, S. N.; Belgi, A.; McArthur, J. R.; Hung, A.; MacRaild, C. A.; Adams, D. J.; Norton, R. S.; Robinson, A. J. ACS Chem. Biol. 2013, 8, 1815). To this end, catalysts Ru-1 and Ru-2 were employed in Z-selective RCM for stapling α-helical peptides that encompass the vast majority of peptides used for biological studies.

As part of the ongoing effort to expand the utility of catalysts Ru-1 and Ru-2, it was chosen to conduct RCM on resin-supported peptides. This would streamline the synthesis of peptides and offer a modular platform to test the activity of cyclometalated ruthenium catalysts. The goal was to compare the activity of Z-selective catalysts to those of non-cyclometalated ruthenium catalysts in RCM and efforts were focused on peptides with known biological activity. The sequence chosen is derived from an α-helical peptide known to target the BCL-2 family of proteins involved in the regulation of apoptosis and whose activity is modulated by constraining the peptide through hydrocarbon stapling (Table 8), (see Walensky, L. D.; Kung, A. L.; Escher, I.; Malia, T. J.; Barbuto, S.; Wright, R. D.; Wagner, G.; Verdine, G. L.; Korsmeyer, S. J. Science 2004, 305, 1466). Peptide 16 is a modified sequence of the known BID peptide used to target the BCL-2 family of proteins. The original peptide sequence was modified to facilate synthesis. The chemical features of peptide 16 consist of two stereochemically defined α,α-disubstituted olefinic amino acids (the non-natural amino acid Fmoc-(S)-2-(4-pentenyl)alanine was incorporated at both i and i+4 residues) separated by one turn of a helix (i.e., olefins positioned at i, i+4 residues) that upon ring closure would generate a 21-membered macrocycle.

TABLE 8
Z-selective RCM to form i, i + 4 stapled peptides

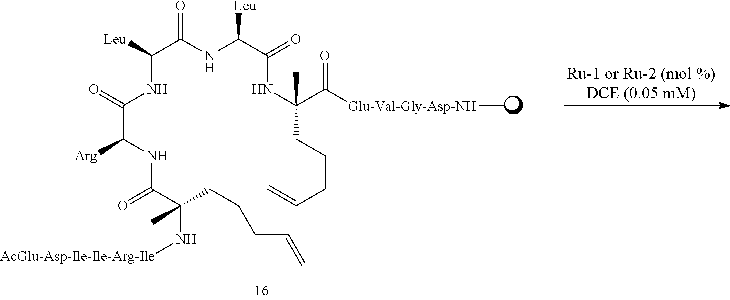

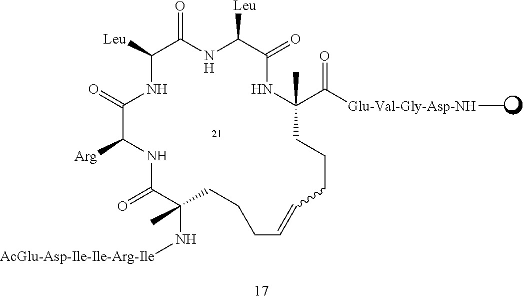

| | cat. | | | Conversion (%)^{b,c} | | Z-selectivity^{d} | |
|---|---|---|---|---|---|---|---|
| Entry | (mol %) | Resin^{a} | Time | Ru-1 | Ru-2 | Ru-1 | Ru-2 |
| 1 | 10 | Wang | 2 h | 25 | 20 | n.d. | n.d. |
| 2 | 10 | TentaGel | 2 h | 40 | 30 | n.d. | n.d. |
| 3 | 10 | MHBA | 2 h | 60 | 55 | n.d. | n.d. |
| 4 | 10 | MBHA | 4 h | 70 | 60 | >85 | >90 |
| 5 | 10 (× 2) | MHBA | 4 h | 75 | 75 | >85 | >90 |
| 6 | 10 (× 2) | MHBA | 4 h | 80^{e} | 70 | >85 | >90 |

^{a}Loading capacities for resin: Wang (0.5 mmol/g); TentaGel (0.25 mmol/g); MBHA (0.5 mmol/g).
^{b}Conversions determined by analytical HPLC of cleaved peptide
^{c}Amino acids were protected prior to RCM
^{d}Determined by analytical HPLC-MS
^{e}Reaction carried out at 40° C.

The stability and activity of catalysts Ru-1 and Ru-2 in the presence of resins was unexplored, and commonly used resins were examined for solid-phase peptide synthesis that varied based on composition and loading capacity. Throughout the experiments, RCM was performed in solvents that promote α-helicity (e.g., dicholorethane, DCE) at concentrations that favor macrocyclic ring closure. Initial screening revealed that the choice of resin influenced the activity of catalysts Ru-1 and Ru-2 in RCM. Conversions to the desired RCM product 17 were typically low on Wang resin using 10 mol % of catalyst at room temperature for 2 hours (entry 1). Resins bearing hydrophilic linkers proved beneficial, affording conversions approaching 40% under the same reaction conditions (entry 2). The use of MBHA resin led to 60% conversion (entry 3) and further optimization was focused on using this resin. The effect of reaction time and catalyst loading on RCM was explored next. Prolonging the reaction led to modest improvements, generating product 17 in 70% conversion with greater than 90% Z-selectivity (entry 4) and subjecting the resin-bound peptide to successive rounds of catalyst (see Kim, Y. W.; Grossmann, T. N.; Verdine, G. L. Nat. Protoc. 2011, 6, 761) resulted in conversions of 75% using two cycles of catalyst addition (entry 5). Increasing the temperature to 40° C. afforded 17 in 80% yield and with greater than 90% Z-selectivity (entry 6). Higher temperatures or conducting RCM under microwave conditions led to catalyst decomposition.

To probe the generality of the method, peptides bearing olefinic amino acids spanning across two turns (i, i+7) of a helix and of varying amino acid sequence (Scheme 4) were investigated.

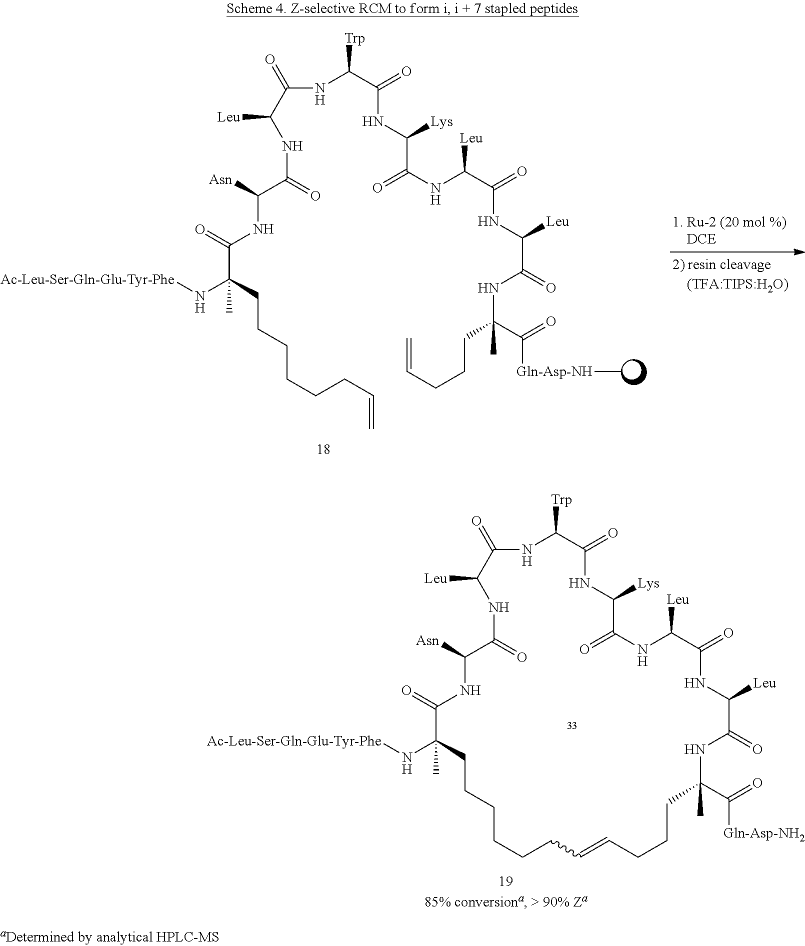

Peptide sequence 18 is derived from a sequence of the tumor suppressor protein p53. To span the distance of two helical turns, the N-terminal olefinic amino acid was modified by increasing the tether length (from five to eight carbon atoms) and inverting the stereochemical configuration (S to R) both of which were predicted to facilitate RCM, (see Schafmeister, C. E.; Po, J.; Verdine, G. L. J. Am. Chem. Soc. 2000, 122, 5891; Bernal, F.; Tyler, A. F. K., S. J. Walensky, L. D. Verdine, G. L. J. Am. Chem. Soc. 2007, 129, 2456; Baek, S.; Kutchukian, P. S.; Verdine, G. L.; Huber, R.; Holak, T. A.; Lee, K. W.; Popowicz, G. M. J. Am. Chem. Soc. 2012, 134, 103). Under these optimized conditions, conversions of 85% to the desired RCM product 19 could be achieved after two cycles of catalyst addition with greater than 90% Z-selectivity. These results demonstrate that cyclometalated ruthenium catalysts can promote Z-selective RCM on solid support for the synthesis of stapled peptides bearing all hydrocarbon crosslinks.

### i, i+3 Z-Selective Ring-Closing Metathesis Attempts on Aib-Containing Peptides

It has been earlier demonstrated that Aib-rich peptides bearing i, i+3 L-serine O-allyl residues afforded highly E-selective RCM products, (see Boal, A. K.; Guryanov, I.; Moretto, A.; Crisma, M.; Lanni, E. L.; Toniolo, C.; Grubbs, R. H.; O'Leary, D. J. J. Am. Chem. Soc. 2007, 129, 6986) in studies motivated, in part, by a theoretical prediction that suggested an RCM-derived 18-membered ring using these side chains would serve as a minimal constraint for the 3₁₀-helix, (see Saviano, M.; Benedetti, E.; Vitale, R. M.; Kaptein, B.; Broxterman, Q. B.; Crisma, M.; Formaggio, F.; Toniolo, C. Macromolecules 2002, 35, 4204). It was investigated whether a Z-selective catalyst could overcome any substrate bias favoring the E-olefin geometry. To this end, the RCM conversion of pentapeptide Boc-L-Ser(Al)-Aib-Aib-L-Ser(Al)-Aib-OMe 20 and heptapeptide Boc-L-Val-L-Ser(Al)-L-Leu-Aib-L-Ser(Al)-L-Val-L-Leu-OMe 24 to macrocycles 21 and 25 using second-generation catalysts 22 and 23 and Z-selective catalyst Ru-1 (Table 9) was investigated.

TABLE 9
RCM to form i, i + 3 stapled peptides

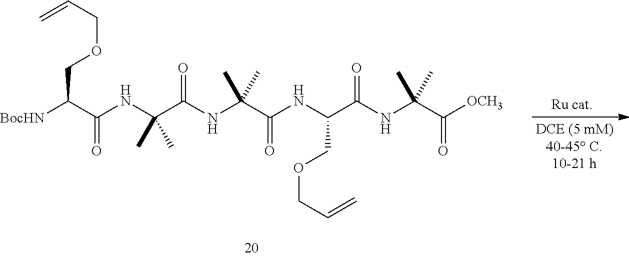

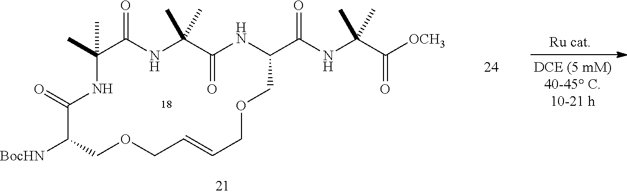

| | | | Cat. | | | Conversion |
|---|---|---|---|---|---|---|
| Entry | Substrate | Product | (mol %) | Temp. | Time | (%)^{a} |
| 1 | 20 | 21 | 22 (10) | 45° C. | 10 h | 86 |
| 2 | | | 23 (10) | 45° C. | 10 h | 85 |
| 3 | | | Ru-1 (30) | 40° C. | 21 h | <5 |
| 4 | 24 | 25 | 22 (10) | 45° C. | 3.5 h | 100 |
| 5 | | | 23 (10) | 45° C. | 3.5 h | 94 |
| 6 | | | Ru-1 (10) | 40° C. | 4 h | <5 |

^{a}Determined by analytical HPLC-MS
^{b}Determined by ¹H NMR spectroscopy

As expected from earlier experiments, peptide 20 readily cyclizes to the E-macrocycle in the presence of 10 mol % 22 or 23 in DCE held at 45° C. for 10 hours (entries 1 and 2). Under similar reactions conditions, no macrocyclization was observed with catalyst Ru-1, even with a three-fold increase in catalyst loading and extended reaction time (entry 3). The same behavior was observed for heptapeptide 24, although in this case the Z-macrocycle does form with 22 or 23 to the extent of ca. 8% (entries 4 and 5). Peptide 24 rapidly cyclized with the second-generation catalysts in refluxing dichloromethane but it was unreactive towards catalyst Ru-1 under identical conditions (entry 6). While most peptide side chain RCM reactions produce E/Z mixtures, the minimal i, i+3 cross-link in these Aib-containing systems seems reluctant to form the Z-olefin, probably a consequence of the conformational restrictions imposed by the Cc-tetrasubstituted α-amino residues. Earlier studies focused mainly on the octapeptide Boc-Aib-Aib-Aib-Ser(O-Allyl)-Aib-Aib-Ser(O-Allyl)-Aib-OMe that afforded highly E-selective (>20:1 E:Z) macrocycles. From these studies, it can be concluded that Aib residues at i+1 and i+2 positions control the E/Z ratio of the RCM product.

### Diastereoselective RCM on Macrocyclic Peptides Bearing i, i+2 Olefin Crosslinks

Despite the therapeutic potential of macrocyclic peptides, they represent a relatively underdeveloped class of compounds due, in part, to their complex structures and limited methods for their synthesis, (see Davies, J. S. J. Pept. Sci. 2003, 9, 471; Katsara, M.; Tselios, T.; Deraos, S.; Deraos, G.; Matsoukas, M. T.; Lazoura, E.; Matsoukas, J.; Apostolopoulos, V. Curr. Med. Chem. 2006, 13, 2221; Jiang, S.; Li, Z.; Ding, K.; Roller, P. P. Curr. Org. Chem. 2008, 12, 1502). RCM was applied as a strategy to streamline the synthesis of cyclic peptides and to investigate the influence of olefin type, position, and size of the macrocycle on the efficiency and stereoselectivity of RCM. Moreover, detailed comparative experiments of a variety of ruthenium catalysts in promoting RCM on peptides (Scheme 5) were conducted.

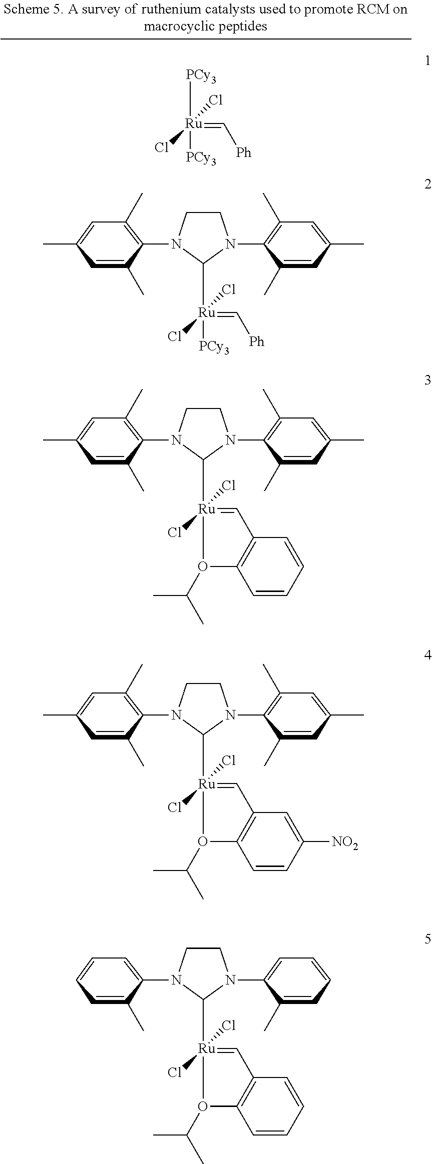

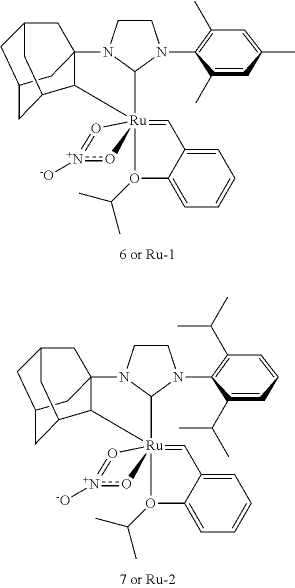

Initial experiments began with the optimization of RCM on dienes 8a-c that contain olefins spanning across i, i+2 residues using ruthenium catalysts 1-5, of Scheme 5 and Z-selective cyclometalated catalysts 6 (Ru-1) and 7 (Ru-2) of Scheme 5 (Table 10). Using this strategy, the intrinsic E/Z stereoselectivity of each catalyst in macrocycle formation was assessed and an understanding of the relative activity of each catalyst to promote RCM was gained.

TABLE 10
Ring-closing metathesis of peptides bearing i, i + 2 olefin crosslinks

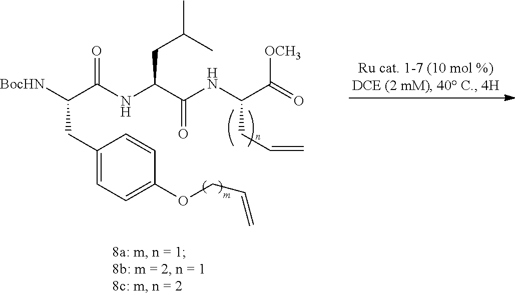

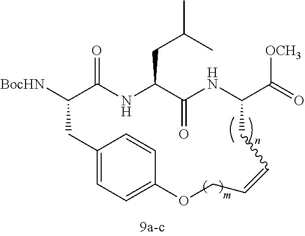

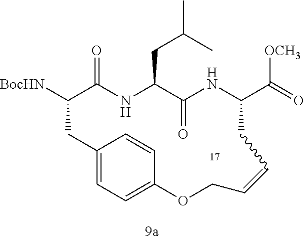 [SMILES: COC(=O)[C@@H]1CC=CCOc2ccc(cc2)C[C@H](NC(=O)OC(C)(C)C)C(=O)N[C@@H](CC(C)C)C(=O)N1]

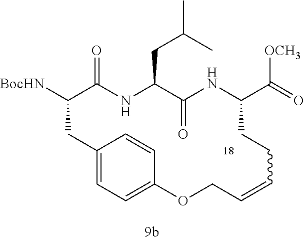 [SMILES: COC(=O)[C@@H]1CCC=CCOc2ccc(cc2)C[C@H](NC(=O)OC(C)(C)C)C(=O)N[C@@H](CC(C)C)C(=O)N1]

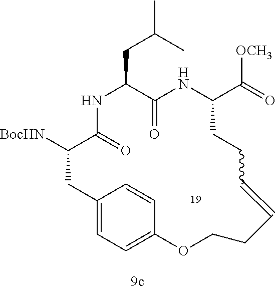 [SMILES: COC(=O)[C@@H]1CCC=CCCOc2ccc(cc2)C[C@H](NC(=O)OC(C)(C)C)C(=O)N[C@@H](CC(C)C)C(=O)N1]

| | Yield (%)^{a} | | | Selectivity (E:Z)^{b} | | |
|---|---|---|---|---|---|---|
| | 9a | 9b | 9c | 9a | 9b | 9c |
| Catalyst | m = 1, n = 1 | m = 2, n = 1 | m = 2, n = 2 | m = 1, n = 1 | m = 2, n = 1 | m = 2, N = 2 |
| 1 | 58 | 54 | 68 | 81:19 | 72:28 | 66:34 |
| 2 | 71 | 61 | 77 | 91:9 | 88:12 | 82:18 |
| 3 | 63 | 55 | 70 | 90:10 | 85:15 | 83:17 |
| 4 | 66 | 60 | 66 | 82:18 | 85:15 | 78:22 |
| 5 | 45 | 24 | 44 | 81:19 | n.d. | 81:19 |
| 6 | 47 | 21 | 26 | 13:87 | n.d. | 15:85 |
| 7 | 41 | 17 | 24 | 7:93 | n.d. | 5:95 |

^{a}Isolated yields
^{b}Determined by ¹H and ¹³C NMR spectroscopy

Exposing diene 8a to the first-generation ruthenium catalyst 1 under dilute conditions to promote macrocycle formation afforded the RCM product 9a in 58% yield and with 80% selectivity for the E-olefin isomer. The use of the more active second-generation catalyst 2 afforded 9a in 71% yield and 90% E-selectivity. The use of chelated isopropoxy catalysts in RCM was next examined. Exposing diene 8a to catalyst 3 afforded macrocycle 9a in 63% yield and with 90% E-selectivity. Comparable yields and diastereoselectivities were observed in the presence of the faster initiating catalyst 4, affording 9a in 66% yield and 82% E-selectivity. In comparing the relative activity of catalysts 1-7 in RCM, the reactions were performed under dilute conditions and at 40 degrees Celsius. Conducting RCM at higher temperatures led to catalyst decomposition of catalysts 6 and 7. The chelated isopropoxy catalysts 3 and 4 have been shown, in many cases, to be more active at higher temperatures.

The use of catalysts bearing less sterically encumbering substituents around the ruthenium center (i.e., tolyl catalyst 5, Stewart, I. C.; Douglas, C. J.; Grubbs, R. H. Org. Lett. 2008, 10, 441) were also explored but conversions to 9a were typically low, affording the product in 45% yield. The lower activity of catalyst 5 in RCM may result from a greater incidence of non-productive olefin metathesis which has been observed with less hindered ruthenium catalysts (see Stewart, I. C.; Douglas, C. J.; Grubbs, R. H. Org. Lett. 2008, 10, 441). The use of Z-selective cyclometalated catalysts 6 and 7 afforded 9a in 47% and 41% yield, respectively. Notably, the olefin selectivity could be reversed to afford macrocycles highly enriched in the Z-olefin isomer.

To probe the influence of macrocycle size on the stereoselectivity of RCM, an additional methylene unit was incorporated into one (i.e., peptide 8b) or both (8c) positions of the olefin-bearing amino acids. It was anticipated that such modifications might influence the E/Z ratio of olefin geometry in the product due to the varying ring sizes that form upon macrocyclization, (see Abell, A. D.; Alexander, N. A.; Aitken, S. G.; Chen, H.; Coxon, J. M.; Jones, M. A.; McNabb, S. B.; Muscroft-Taylor, A. J. Org. Chem. 2009, 74, 4354.)

Moreover, whether the identity of the olefin (i.e., allylic or homoallylic) had any influence on the efficiency of RCM using catalysts 1-7 was determined. Exposing substrate 8b to catalysts 1-5 resulted in variable yields and E/Z ratios for the formation of macrocycle 9b, from 54% yield and 70% diastereoselectivity for catalyst 1 to 24% yield and 80% E-selectivity for catalyst 5. The cyclometalated ruthenium catalysts 6 and 7 were less active than catalysts 1-5 in RCM of 8b, with conversions below 25% for the formation of 9b. Interestingly, formation of the 18-membered macrocycle 9b was consistently lower than formation of 9a, (competing dimerization of 8b (ca. ˜15%) which may account for the lower yield of the desired RCM product) and this finding prompted the examination of the structurally analogous peptide 8c, bearing an additional methylene that would give rise to the 19-membered macrocycle 9c upon RCM. In general, the conversion of peptide 8c to macrocycle 9c (66-77%) was improved relative to the conversion of 8b to macrocycle 9b (50-60%). In the presence of the first-generation catalyst 1, the selectivity for the E-isomer was lower for 9c (66%) compared to 9a (81%) and 9b (77%) and this trend was consistent with catalyst 2 in RCM. As with macrocycles 9a and 9b, catalysts 3 and 4 were more active than catalyst 5 in RCM, affording the desired macrocycle 9c in 70% yield and 80% E-olefin selectivity as compared to 44% yield and 80% selectivity in the presence of catalyst 5. Interestingly, the propensity of macrocycles 9b and 9c to form with greater Z-selectivity relative to 9a using non-selective catalysts 1-5 did not facilitate RCM in the presence of Z-selective catalysts 6 and 7 as shown by the comparatively low yields of 9b (21%) and 9c (26%) to 9a (41%) with 6 or 7. This finding is consistent with previous reports suggesting that cyclometalated ruthenium catalysts, in some cases, cannot overcome any substrate bias that may favor the formation of Z-olefin geometry during metathesis, (see Mangold, S. L.; O'leary, D. J.; Grubbs, R. H. J. Am. Chem. Soc. 2014, 136, 12469). These studies provide evidence that subtle variations of catalyst structure and macrocycle size can greatly impact the yield and diastereoselectivity of RCM on olefin-bearing peptides. For macrocycles bearing homoallylic olefin tethers (i.e., 9c), the E/Z diastereoselectivity of RCM was generally lower than for dienes consisting of allylic olefin tethers (i.e., 9a). In this regard, macrocyclization of 9c was improved relative to 9a, mostly notably in the presence of phosphine-containing catalysts 1 and 2.

### The Influence of Heteroatoms and Peptide Sequence in Stereoselective RCM on Peptides Bearing i, i+3 Olefin Crosslinks

The experiments regarding the activity of catalysts 1-7 of Scheme 5, in RCM on substrates 8a-c suggests that the size of the macrocycle can influence E/Z diastereoselectivity. To explore this further, peptides bearing an additional amino acid between olefin crosslinks were synthesized. This would enable access to additional cyclic structures and provide insight into the effect of varying the position of olefin-containing amino acids along the peptide in RCM. Moreover, a larger variety of amino acids were investigated, including those bearing allylic heteroatoms in the side chain, in macrocyclic ring closure. These studies were motivated by the observation that both the peptide sequence and identity of the olefin can profoundly affect the efficiency of metathesis in homodimerization and cross metathesis on peptides and whether these trends extended to RCM were investigated, (see Mangold, S. L.; O'leary, D. J.; Grubbs, R. H. J. Am. Chem. Soc. 2014, 136, 12469).

The influence of allylic heteroatoms in facilitating RCM and their influence on E/Z diastereoselectivity was first evaluated. Exposing the allyl-modified peptide 10 to the optimized reaction conditions afforded macrocycle 15 in 70% yield with 74% E-selectivity using catalyst 1 (Table 11). By comparison, the O-allyl serine (11) and S-allyl cysteine (12) modified peptides gave the desired macrocycles 16 and 17 in 73% and 77% yield and with 92% and 90% E-selectivity, respectively. The higher yields of macrocycles 16 and 17 relative to 15 could also result from the formation of a larger ring size (18) relative to 15 (14) in addition to enhancing RCM through an allylic heteroatom effect.

These trends were observed in the presence of the second-generation ruthenium catalyst 2; in this instance, the formation of macrocycle 15 could be obtained in 74% yield, as compared to macrocycles 16 (76%) and 17 (78%) in RCM. Peptides 10-12 were exposed next to catalysts 3 and 4. The O-allyl modified peptide 11 was converted to macrocycle 16 in 73% yield, compared to 61% yield for the conversion of allyl peptide 10 to 15 using catalyst 3. Similar yields and selectivities were observed with catalyst 4, whereby peptide 11 afforded slightly higher yields of the RCM product 16 (74%) relative to the conversion of 10 to 15 (64%). The allyl cysteine-modified peptide 12 was exposed next to RCM. The formation of macrocycle 17 was generally improved relative to 15 or 16, occurring in 80% yield and with 90% E-selectivity using catalysts 1-3. As observed with substrates 8a-c, the use of tolyl catalyst 5 under the RCM conditions led to lower conversions to macrocycles 15-17 (39-44%). By comparison, the use of Z-selective catalyst 6 and 7 in RCM resulted in yields ranging from 32-40% for formation of 15-17 but with reversal of olefin selectivity. Unlike with the use of isopropoxy catalysts 3 and 4 in RCM, an absence of a pronounced heteroatom effect was observed with cyclometalated catalysts 6 and 7 that may be attributable to their comparatively lower reactivity in RCM.

TABLE 11
Ring-closing metathesis on peptides bearing i, i + 3 olefin crosslinks

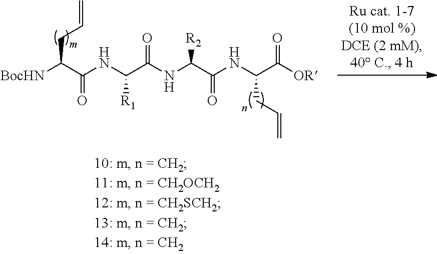

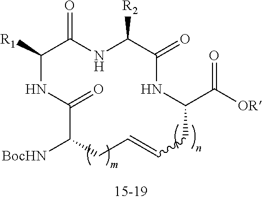

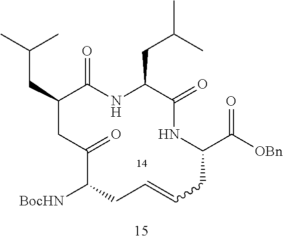 [SMILES: CC(C)C[C@@H]1CC(=O)[C@@H](NC(=O)OC(C)(C)C)CC=CC[C@@H](C(=O)OCc2ccccc2)NC(=O)[C@H](CC(C)C)NC1=O]

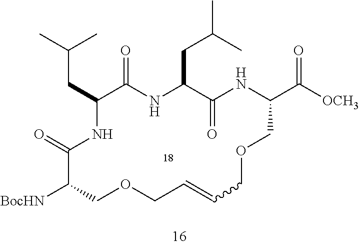 [SMILES: COC(=O)[C@@H]1COCC=CCOC[C@H](NC(=O)OC(C)(C)C)C(=O)N[C@@H](CC(C)C)C(=O)N[C@@H](CC(C)C)C(=O)N1]

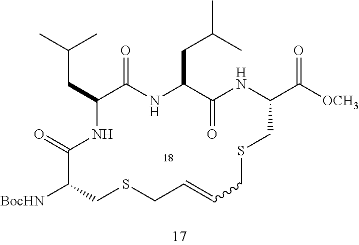 [SMILES: COC(=O)[C@@H]1CSCC=CCSC[C@H](NC(=O)OC(C)(C)C)C(=O)N[C@@H](CC(C)C)C(=O)N[C@@H](CC(C)C)C(=O)N1]

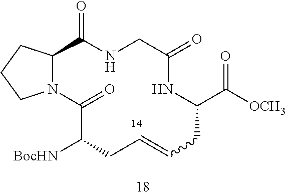 [SMILES: COC(=O)[C@@H]1CC=CC[C@H](NC(=O)OC(C)(C)C)C(=O)N2CCC[C@H]2C(=O)NCC(=O)N1]

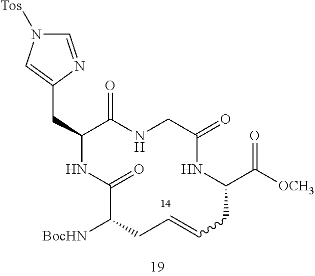

| | Yield (%)^{a} | | | | | Selectivity (E:Z)^{b} | | | | |
|---|---|---|---|---|---|---|---|---|---|---|
| Catalyst | 15 | 16 | 17 | 18 | 19 | 15 | 16 | 17 | 18 | 19 |
| 1 | 70 | 73 | 77 | 37 | 14 | 74:27 | 92:8 | 90:10 | 77:23 | 80:20 |
| 2 | 74 | 76 | 81 | 48 | 18 | 90:10 | 93:7 | 92:8 | 91:9 | 89:11 |
| 3 | 61 | 73 | 79 | 41 | <10 | 86:14 | 90:10 | 90:10 | 80:20 | n.d |
| 4 | 64 | 74 | 83 | 36 | 12 | 82:18 | 85:15 | 85:15 | 82:18 | n.d |
| 5 | 39 | 44 | 42 | 20 | <5 | 85:15 | 91:9 | 91:9 | 81:19 | n.d |
| 6 | 32 | 36 | 40 | 17 | <5 | 12:88 | 13:87 | 13:87 | n.d | n.d |
| 7 | 30 | 34 | 33 | 18 | <5 | 10:90 | 5:95 | 5:95 | n.d | n.d |

^{a}Isolated yield
^{b}Determined by ¹H and ¹³C NMR spectroscopy

Peptides of varying amino acid sequence in RCM were examined next. Previous studies regarding the activity of catalysts 6 and 7 in homodimerization and cross metathesis revealed that a subset of olefin-bearing amino acids had a deactivating effect on olefin metathesis, (see Mangold, S. L.; O'leary, D. J.; Grubbs, R. H. J. Am. Chem. Soc. 2014, 136, 12469). Specifically glycine, proline, and histidine were shown to be unreactive in homodimerization and cross metathesis. To test whether such amino acids generally inhibit metathesis by using a broader range of catalysts and whether incorporation of these amino acids within a larger peptide could override their apparent inactivity, peptide 13 was generated containing the amino acids proline and glycine at positions along the peptide proximal to the olefin-bearing amino acids. In the presence of catalysts 6 and 7, conversions of 13 to 18 were less than 20%. For comparison, catalysts 1-5 in RCM on diene 13 were examined. Yields to the corresponding macrocycle 18 were variable, ranging from 20% in the presence of catalyst 5 to 48% with catalyst 2. For those catalysts that could achieve reasonable yields of 22, the selectivity was above 80% in favor of the E-olefin isomer. As a further test, peptide 14 was synthesized bearing histidine in place of proline and its activity in RCM was evaluated. Conversions of 14 to macrocycle 19 were consistently below 25% in the presence of catalysts 1-7. Under these conditions, formation of the 14-membered ring may be hindered by the proximity of histidine, (dimerization ca. ˜10% in addition to a small percentage of the RCM product for peptide 14 was observed) which has been shown to have a deactivating effect on metathesis activity, (see Chapman, R. N.; Arora, P. S. Org. Lett. 2006, 8, 5825). Taken together, such results point to the importance of olefin identity, peptide sequence, and catalyst structure in controlling both the efficiency and diastereoselectivity of RCM on macrocyclic peptides. For those peptides bearing i, i+3 olefin crosslinks, incorporation of allylic heteroatoms into the amino acid side-chain generally favored RCM, most notably in the presence of isopropoxy catalysts 3 and 4 and to a lesser extent with phosphine containing catalysts 1 and 2 and cyclometalated ruthenium catalysts 6 and 7. These observations reflect the importance of directly comparing various catalyst structures in RCM and guide further strategies for optimizing olefin metathesis on peptide-containing substrates.

### Z-Selective Ethenolysis for the Enrichment of Macrocyclic Peptides in E-Olefin Geometry

Encouraged by the success of RCM on a variety of peptide substrates, strategies to transform macrocyclic peptides having a mixture of olefin isomers into those bearing a single olefin isomer were evaluated next. Such a strategy could facilitate the isolation and characterization of olefin-containing macrocycles and offer a means to more easily investigate the influence of olefin geometry on the stability, activity, and conformation of this important class of compounds.

An olefin enrichment strategy using a catalyst-controlled ethenolysis pathway (Scheme 6) was envisoned. This approach capitalizes on the inherent reversibility of olefin metathesis by using ethylene to drive ring-opening metathesis, (see Marinescu, S. C.; Levine, D. S.; Zhao, Y.; Schrock, R. R.; Hoveyda, A. H. J. Am. Chem. Soc. 2011, 133, 11512; Miyazaki, H.; Herbert, M. B.; Liu, P.; Dong, X.; Xu, X.; Keitz, B. K.; Ung, T.; Mkrtumyan, G.; Houk, K. N.; Grubbs, R. H. J. Am. Chem. Soc. 2013, 135, 5848). By having a catalyst that is selective for the formation of one olefin isomer (e.g., catalysts 6 and 7) it should be possible to selectivity degrade olefin isomers from the corresponding mixtures. This strategy could serve as a valuable tool to form cyclic peptides having a single olefin isomer which, to date, has been a synthetic challenge using olefin metathesis.

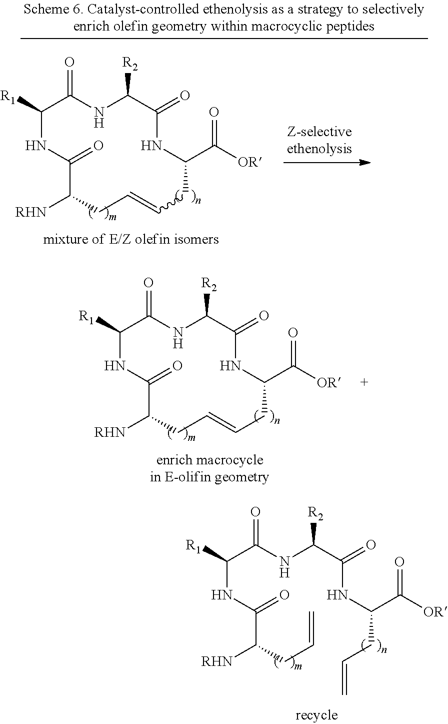

For the initial experiments, catalyst 6 (Ru-1) was examined in Z-selective ethenolysis using macrocycles bearing i, i+2 or i, i+3 crosslinks and having variable ratios of olefin isomers. In this way, it was determined if the E/Z ratio in macrocyclic peptides affect the efficiency of ethenolysis. Under the optimized ethenolysis conditions, nearly complete Z-degradation of substrate 9a occurred in the presence of ethylene (1 atm) and catalyst 6 (Ru-1) (5 mol %), affording enrichment of 9a in the E-olefin isomer in greater than 98% (entry 1, Table 12). Some E-degradation (ca. ˜12%) occurred after prolonged exposure to the ethenolysis conditions. More significantly, the ethenolysis conditions were able to transform macrocycle 9b from a 80:20 mixture of isomers to those bearing almost exclusive formation of the E-isomer (entry 2). To test the generality of the method, these conditions were applied to macrocyclic peptides 9c and 15-18. Complete consumption of the Z-isomer was observed for 9c and 15-17, affording the pure E macrocycles with enrichment above 98% (entries 3-6). A notable exception was compound 18 that resulted in comparatively low enrichment (entry 7). The relatively low percentage of E-olefin enrichment for compound 18 may be partially attributed to its lower reactivity in RCM. From these studies, the efficiency of Z-selective ethenolysis does not appear to be greatly influenced by the initial E/Z ratio of olefins in macrocycles 9 and 15-18.

TABLE 12
Catalyst-controlled ethenolysis for the enrichment of
olefin geometry in macrocyclic peptides

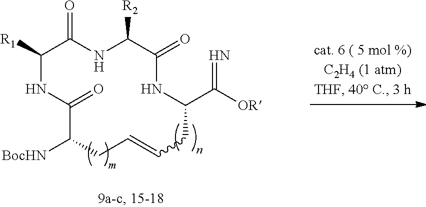

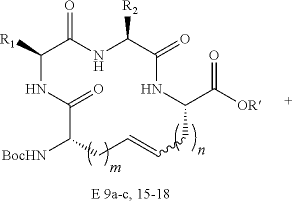

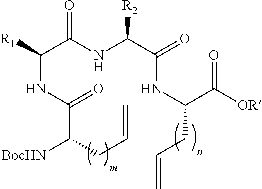

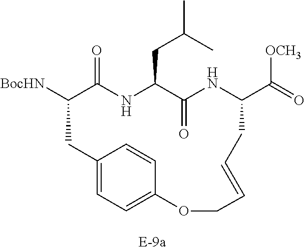 [SMILES: COC(=O)[C@@H]1C/C=C/COc2ccc(cc2)C[C@H](NC(=O)OC(C)(C)C)C(=O)N[C@@H](CC(C)C)C(=O)N1]

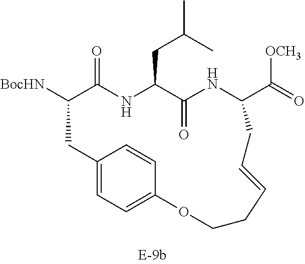 [SMILES: COC(=O)[C@@H]1C/C=C/CCOc2ccc(cc2)C[C@H](NC(=O)OC(C)(C)C)C(=O)N[C@@H](CC(C)C)C(=O)N1]

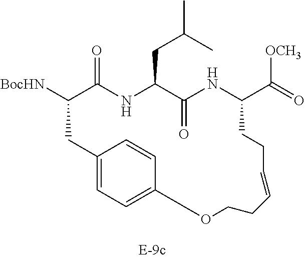 [SMILES: COC(=O)[C@@H]1CC/C=C/CCOc2ccc(cc2)C[C@H](NC(=O)OC(C)(C)C)C(=O)N[C@@H](CC(C)C)C(=O)N1]

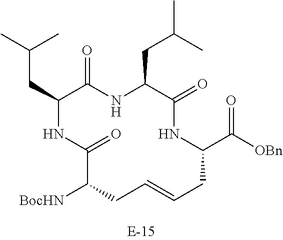 [SMILES: CC(C)C[C@@H]1NC(=O)[C@@H](NC(=O)OC(C)(C)C)C/C=C/C[C@@H](C(=O)OCc2ccccc2)NC(=O)[C@H](CC(C)C)NC1=O]

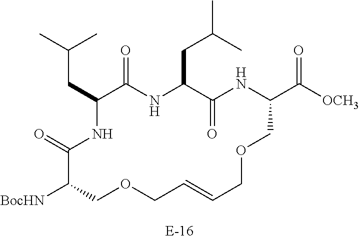

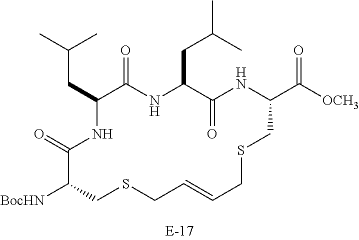

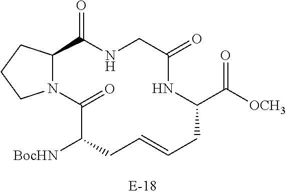 [SMILES: COC(=O)[C@@H]1C/C=C/C[C@H](NC(=O)OC(C)(C)C)C(=O)N2CCC[C@H]2C(=O)NCC(=O)N1]

| Entry | Compound | Initial E:Z^{a} | Final E:Z^{a} | Yield^{b} |
|---|---|---|---|---|
| 1 | 9a | 96:4 | 99:1 | 62 |
| 2 | 9b | 80:20 | 97:3 | 74 |
| 3 | 9c | 82:18 | 96:4 | 80 |
| 4 | 15 | 90:10 | >99:1 | 77 |
| 5 | 16 | 81:19 | 98:2 | 64 |
| 6 | 17 | 90:10 | 99:1 | 67 |
| 7 | 18 | 77:23 | 88:12 | 45 |

^{a}Determined by ¹H, ¹³C NMR spectroscopy and analytical HPLC/MS
^{b}Isolated yield

For those peptides that underwent efficient ethenolysis, the resulting starting material could be recovered and resubjected to the RCM conditions, providing a method for increasing the overall yield of product through iterative RCM/ethenolysis metathesis events. These results suggest that catalyst-controlled ethenolysis can serve as a practical method for the selective formation of E-olefins in macrocyclic peptides. This strategy, when coupled to Z-selective RCM can afford macrocycles predominantly enriched in E or Z olefin geometry.

### RCM of Resin-Bound α-Helical Peptides Bearing i, i+4 and i, i+7 Crosslinks

The investigation of macrocyclic ring closure on peptides containing i, i+2 or i, i+3 olefinic crosslinks revealed that the peptide sequence and olefin identity can influence the efficiency and diastereoselectivity of RCM. Moreover, macrocycles consisting of a mixture of olefin isomers could be enriched in E-olefin geometry using Z-selective ethenolysis and that the initial E/Z ratio did not appear to influence the efficiency of ethenolysis. It was sought to further expand these studies to α-helical peptides bearing i, i+4 or i, i+7 olefinic tethers. Such compounds, often referred to as stapled peptides, (see Miller, S. J.; Blackwell, H. E.; Grubbs, R. H. J. Am. Chem. Soc. 1996, 118, 9606; Blackwell, H. B.; Grubbs, R. H. Angew. Chem. Int. Ed. 1998, 37, 3281; Schafmeister, C. E.; Po, J.; Verdine, G. L. J. Am. Chem. Soc. 2000, 122, 5891) have gained attention as potential therapeutics in a variety of areas including cancer, infectious disease, and metabolism, (see Walensky, L. D.; Bird, G. H. J. Med. Chem. 2014, 57, 6275; Verdine, G. L.; Hilinski, G. J. Methods Enzymol. 2012, 503, 3; Bird, G. H.; Gavathiotis, E.; LaBelle, J. L.; Katz, S. G.; Walensky, L. D. ACS Chem. Biol. 2014, 9, 831; Kim, Y. W.; Grossmann, T. N.; Verdine, G. L. Nat. Protoc. 2011, 6, 761).

TABLE 13
RCM on peptides bearing i, i + 4 and i, i + 7 olefin crosslinks

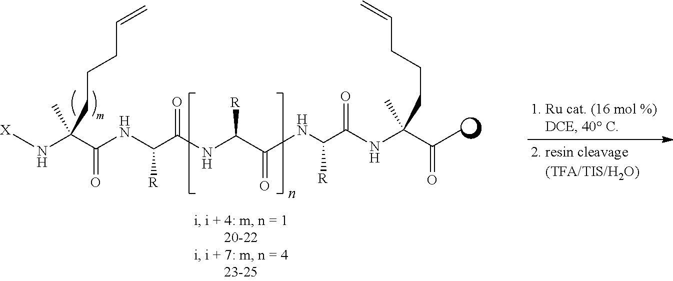

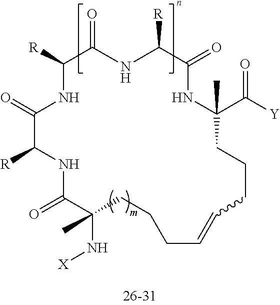

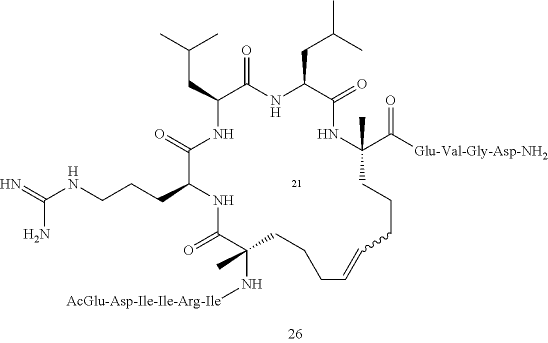

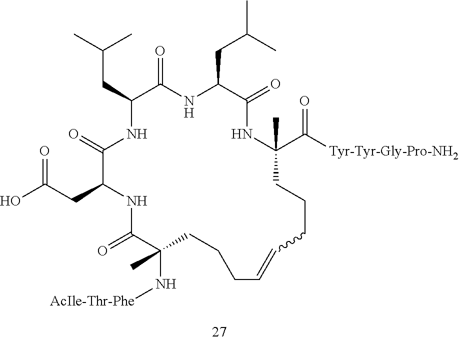

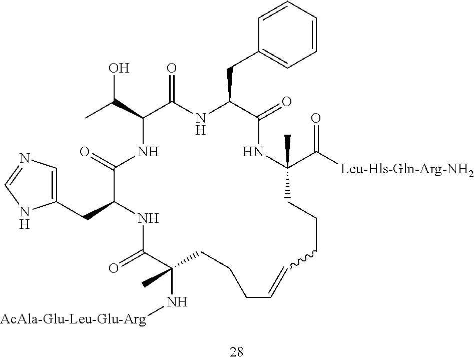

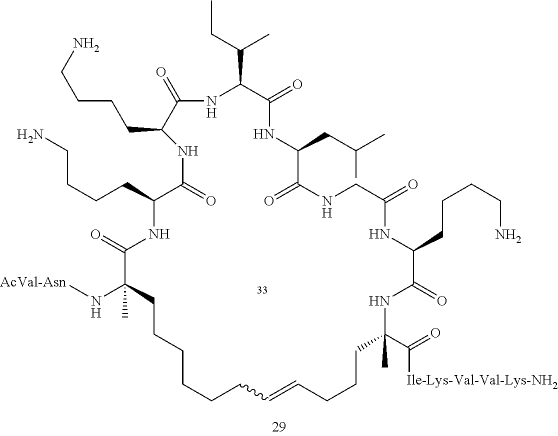

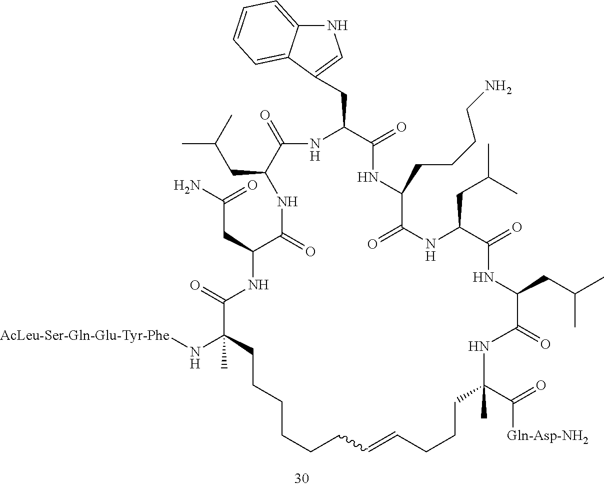

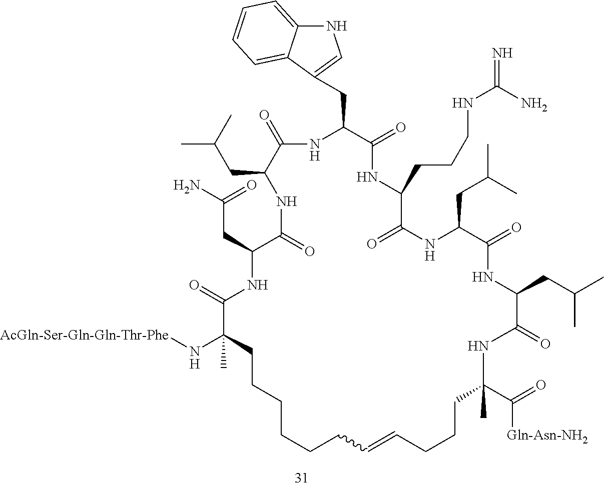

| | 26 | 27 | 28 | 29 | 30 | 31 |
|---|---|---|---|---|---|---|
| Catalyst | m, n = 1 | m, n = 1 | m, n = 1 | m, n = 4 | m, n = 4 | m, n = 4 |
| | Conversion (%)^{a} | | | | | |
| 1 | 94 | 90 | 93 | 88 | 96 | 92 |
| 2 | 97 | 90 | 95 | 94 | 95 | 90 |
| 3 | 84 | 81 | 88 | 80 | 84 | 85 |
| 4 | 88 | 80 | 92 | 85 | 88 | 85 |
| 5 | 76 | 70 | 70 | 60 | 84 | 80 |
| 6 | 83 | 75 | 80 | 70 | 80 | 75 |
| 7 | 81 | 75 | 70 | 55 | 75 | 70 |
| | Selectivity (E:Z)^{a} | | | | | |
| 1 | 66:23 | 64:36 | 70:30 | 58:42 | 62:38 | 65:35 |
| 2 | 80:20 | 75:25 | 83:17 | 80:20 | 75:25 | 79:21 |
| 3 | 80:20 | 75:25 | 74:26 | 78:22 | 78:22 | 80:20 |
| 4 | 72:28 | 74:26 | 72:28 | 71:29 | 81:19 | 81:19 |
| 5 | 66:33 | 70:30 | 74:26 | n.d. | 71:29 | 74:26 |
| 6 | 20:80 | 22:78 | 18:82 | 20:80 | 21:79 | 17:83 |
| 7 | 17:83 | 19:81 | 23:77 | n.d. | 18:82 | 20:80 |

^{a}Determined by analytical HPLC-MS

To date, most strategies for macrocyclization on α-helical peptides rely on RCM and subsequent hydrogenation to generate a fully saturated hydrocarbon tether along the peptide helix, (see Kim, Y. W.; Grossmann, T. N.; Verdine, G. L. Nat. Protoc. 2011, 6, 761; Bird, G. H.; Crannell, W. C.; Walensky, L. D. Curr. Protoc. Chem. Biol. 2011, 3, 99). In this sense, little attention has been focused on examining the role of olefin geometry on the conformation or biological activity of macrocyclic peptides. Whether the RCM/ethenolysis reaction manifold could provide a method to synthesize stapled peptides with defined olefin geometry was explored. Moreover, it was sought to extend the methodology to peptides on resin for enabling a more streamlined and high-throughput method of peptide synthesis, identification, and purification. In choosing the peptide sequences, focus was placed on those having a variety of amino acids and olefin crosslinks and experiments began with the optimization of RCM on peptides bearing i, i+4 crosslinks that afford a 21-membered macrocycle (Table 13). RCM on resin-bound peptide 20 was first evaluated, (see Kim, Y. W.; Grossmann, T. N.; Verdine, G. L. Nat. Protoc. 2011, 6, 761) using the first-generation catalyst 1. After extensive optimization, conversions to the desired cyclic peptide 26 could be achieved in 94% conversion and with 66% E-selectivity. By comparison, the second-generation catalyst 2 afforded 26 in 97% conversion and 80% E-selectivity under the same reaction conditions. Exposing 20 to catalysts 3 and 4 led to conversions of 84% and 88%, respectively. As observed with other olefin-containing peptides, the tolyl catalyst 5 was less active in RCM, with 75% conversion of 20 to 26. Applying the cyclometalated catalysts 6 and 7 to the RCM conditions afforded 26 in 83% and 81% conversion, respectively. In these instances, the selectivity was in favor of the Z-olefin isomer. Peptides containing the amino acids proline (competing dimerization of 8b (ca. -15%) which may account for the lower yield of the desired RCM product) and histidine were also examined, (see Zhang, H.; Zhao, Q.; Bhattacharya, S.; Waheed, A. A.; Tong, X.; Hong, A.; Heck, S.; Curreli, F.; Goger, M.; Cowbum, D.; Freed, E. O.; Debnath, A. K. J. Mol. Biol. 2008, 378, 565) that were shown to reduce the efficiency of RCM on peptides bearing i, i+3 olefin crosslinks (i.e., peptides 13 and 14). Incorporating olefin tethers at the i, i+4 positions might facilitate RCM on-resin by serving to preorganize the reactive side chains on the same face of the α-helix. Such preorganization of the olefins, in addition to expanding the size of the macrocycle, might favor RCM over competing deactivation by amino acid side chains. To test this, peptides containing proline and histidine at positions distal from the olefin crosslinks (i.e., peptide 21) or proximal to the crosslinks (22) were synthesized and their activity in RCM was examined. Exposing peptide 21 to catalysts 1 and 2 led to nearly full conversion (90%) of 21 to macrocycle 27. The use of catalysts 3 and 4 in RCM of 21 resulted in slightly lower conversion (80%) and with 75% E-selectivity. Exposing 21 to catalyst 5 afforded macrocycle 27 in 70% conversion, comparable to that of catalysts 6 and 7 (75%). To probe further the role of amino acid sequence in RCM of peptides bearing i, i+4 crosslinks, the histidine-containing peptide 22 was exposed to similar reaction conditions. In the presence of catalysts 1-4 conversions to macrocycle 28 ranged from 80% with catalyst 4 to 90% in the presence of catalyst 1. For these cases, the diastereoselectivity of macrocycle formation ranged from 62% in the presence of 1 to 80% E-selectivity in the presence of catalysts 2-4. A slight decrease in conversion to 28 was seen in the presence of catalysts 6 (Ru-1) and 7 (Ru-2) but with reversal of olefin selectivity. These results imply that the efficiencies of RCM on resin-bound peptides 26-28 are comparable, even for wide variation in peptide sequence.

α-Helical peptides bearing i, i+7 olefin crosslinks were evaluated next for the formation of 33-membered macrocycles, as the goal was to compare the influence of macrocycle size on olefin diastereoselectivity in RCM for resin-bound peptides. For the initial experiments, the conversion of peptide 23 to macrocycle 29 was monitored, (see Moellering, R. E.; Comejo, M.; Davis, T. N.; Del Bianco, C.; Aster, J. C.; Blacklow, S. C.; Kung, A. L.; Gilliland, D. G.; Verdine, G. L.; Bradner, J. E. Nature 2009, 462, 182), using the first- and second-generation ruthenium catalysts 1 and 2. The conversion to macrocycle 29 was achieved in 88% in the presence of 1 and 94% with the use of catalyst 2, respectively. Under these conditions, the selectivity of the E-olefin was only 58% in the presence of 1 but increased to 80% in the presence of catalyst 2. The use of isopropoxy catalysts 3 and 4 afforded 29 in 3:1 E:Z selectivity at 80% conversion, trends that were similar to peptides bearing i, i+4 crosslinks. By comparison, the conversions were typically lower in the presence of catalyst 5 (80%), 6 (Ru-1) (83%) and 7 (Ru-2) (81%). As observed in the formation of macrocycles bearing i, i+2 or i, i+4 crosslinks, the ability to form macrocycles with greater Z-olefin content using non-selective catalysts 1-5 did not facilitate RCM in the presence of Z-selective catalysts 6 (Ru-1) and 7 (Ru-2). As further validation, the use of RCM for formation of macrocycles 30 and 31 was examined. Conversions of 24, (see Chapuis, H.; Slaninova, J.;

Bednarova, L.; Monincova, L.; Budesinsky, M.; Cerovsky, V. Amino Acids 2012, 43, 2047) to 30 reached a maximum of 95% with catalyst 1 with slightly lower conversions in the presence of 2 (90%), 3 (84%) or 4 (86%). In these cases, the selectivity ranged from 60% with 1 to 80% in favor of the E-isomer with the use of catalyst 4. These trends were observed in the formation of macrocycle 31 using catalysts 1-4, with conversions greater than 85% and comparable diastereoselectivity. The use of catalysts 5-7 in macrocyclization of 25, (see Mangold, S. L.; ′'leary, D. J.; Grubbs, R. H. J. Am. Chem. Soc. 2014, 136, 12469) afforded the desired cyclic peptide 31 with slightly lower conversions (60-70%) relative to the formation of macrocycle 30 (75-80%). These comparative studies suggest that increasing the macrocycle size and/or preorganizing the olefins on the same face of the α-helix may facilitate RCM even in the presence of amino acids that normally reduce the efficiency of olefin metathesis. Interestingly, such trends seem be consistent in the presence of phosphine-containing catalysts 1 and 2 or isopropoxy catalysts 3-7.

### Z-Selective Ethenolysis on Resin-Bound α-Helical Peptides

The results regarding RCM on a variety of olefin-bearing peptides revealed that the diastereoselectivity of macrocyclic ring closure was dictated both by the choice of catalyst and size of the macrocycle. In cases involving peptides bearing i, i+2 or i, i+3 olefin crosslinks, RCM generally favored the formation of the E-isomer (˜80% E) in the presence of catalysts 1-5. Alternatively, the use of cyclometalated catalyst 6 or 7 gave rise to macrocycles predominantly of Z-olefin geometry, but at lower yields or conversions as expected for substrates where the E-isomer is normally favored. For macrocycles 8 and 15-18, Z-selective ethenolysis provided a method for further enrichment of E-olefin geometry. While these studies provide a framework for enabling the formation of E or Z olefins in cyclic peptides, it was sought to extend the studies of ethenolysis to resin-bound peptides. Such experiments would prove particularly useful as the diastereoselectivity of RCM to form macrocycles 26-31 was typically low. In this sense, the ability to selectively perform ethenolysis on these macrocycles could streamline methods for their identification and purification.

The initial experiments began with Z-selective ethenolysis on macrocycle 26 (Table 14). Conversion of 26 to the olefin-enriched macrocycle E-26 occurred in 86%, transforming 26 from an initial ratio of 72% E-olefin to greater than 90% E (entry 1). This trend was observed for the selective ethenolysis of macrocycle 27 which occurred in 65% conversion and transformed a 64% mixture of E/Z isomers of 27 to a macrocycle having greater than 95% selectivity for the E-olefin (entry 2). To probe the general utility of the method, macrocycles 29-31 containing i, i+7 crosslinks were exposed to the ethenolysis conditions (entries 4-6). Conversions to the enriched macrocycles varied from 93% for 29 to 78% for the formation of 31. As with macrocycles 26-28, enrichment of 29-31 to the E-olefin could occur in greater than 90%. These studies, in parallel with ethenolysis on macrocycles 8 and 15-18, point to the utility of RCM and ethenolysis as a practical means of olefin enrichment in cyclic peptides.

TABLE 14
Z-selective ethenolysis on stapled peptides
bearing i, i + 4 and i, i + 7 crosslinks

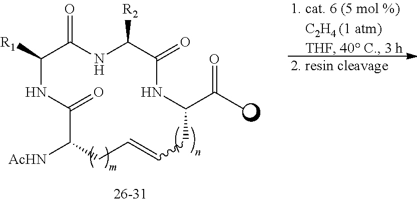

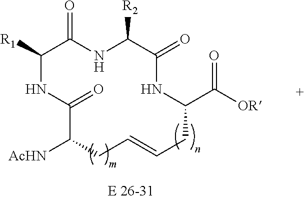

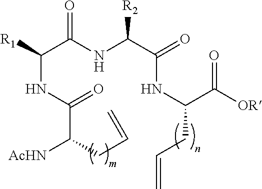

| | Com- | | | |
|---|---|---|---|---|
| Entry | pound | Initial E:Z^{a} | Final E:Z^{a} | Conversion %^{a} |
| 1 | 26 | 72:28 | 95:5 | 86% |
| 2 | 27 | 83:17 | 93:7 | 65% |
| 3 | 28 | 71:29 | 96:4 | 91% |
| 4 | 29 | 79:21 | 94:6 | 78% |
| 5 | 30 | 64:36 | 98:2 | 83% |
| 6 | 31 | 81:19 | 98:2 | 93% |

^{a}Determined by analytical HPLC/MS of cleaved peptide

### Assessing the Role of Olefin Geometry on the Conformation of α-Helical Peptides

Experiments of RCM in tandem with catalyst-directed ethenolysis provided access to macrocyclic peptides enriched in E- or Z-olefin isomers. Whether changes in olefin geometry induced measureable differences in the overall fold or conformation of macrocyclic peptides was examined. For the initial experiments, the α-helical content between non-stapled peptide 21 and the corresponding E or Z macrocycle 27 using circular dichroism (Table 15) was examined. The linear peptide 21 was measured to have an α-helical content of 21% (entry 1) which increased upon macrocyclization to 27 affording an α-helicity of 80% for the E-olefin and 71% for the Z-olefin, respectively (entries 2 and 3). These results are in agreement with the observation that macrocyclization by RCM generally induces greater α-helicity within stapled peptides, (see Walensky, L. D.; Bird, G. H. J. Med. Chem. 2014, 57, 6275; Verdine, G. L.; Hilinski, G. J. Methods Enzymol. 2012, 503, 3; Estieu-Gionnet, K.; Guichard, G. Exp. Opin. Drug Discov. 2011, 6, 937; Bemal, F.; Katz, S. G. Methods Mol. Biol. 2014, 1176, 107). To further expand these experiments, the role of olefin-geometry on the α-helical content within a larger macrocycle was examined next and peptide 23 containing olefins at i, i+7 positions was chosen. For this peptide, the helical content was 7.5% for the non-cyclized peptide (entry 4) but upon macrocyclization to 29, the α-helicity increased to 21% and 23% for the E- and Z-olefin isomers, respectively (entries 5, 6). As observed with macrocycle 27, the difference in the helicity between the Z- and E-olefin isomers in 29 was minimal, suggesting that the olefin geometry in 27 and 29 does not contribute substantially to the overall secondary structure of the macrocycles bearing olefin tethers of such lengths. These experiments informed further explorations into examining the role of olefin geometry on the stability or biological activity of macrocyclic compounds.

TABLE 15
Assessment of olefin geometry on α-helicity of linear peptides 21 and 23 and
corresponding macrocycles 27 and 29

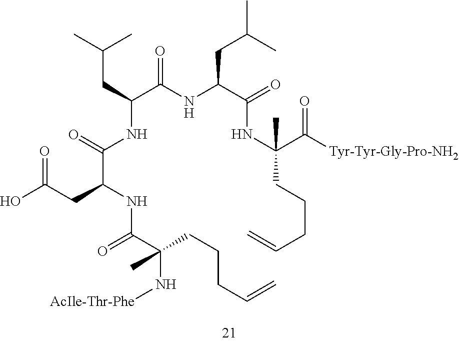

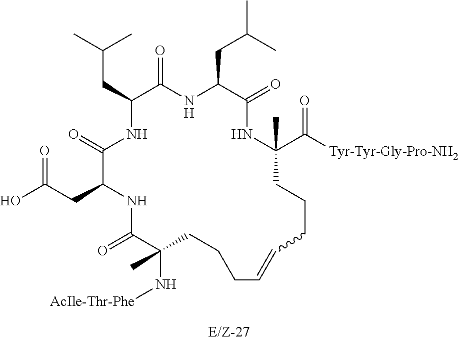

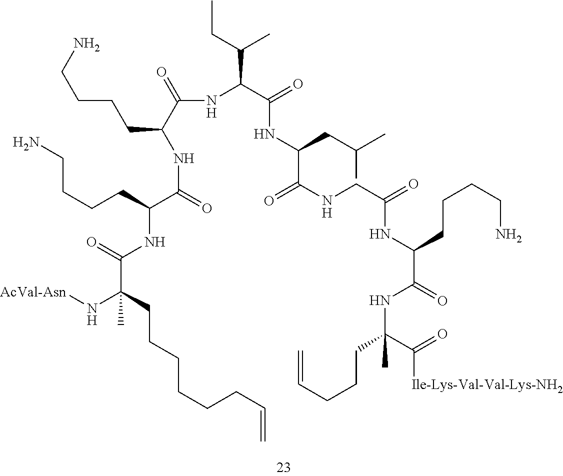

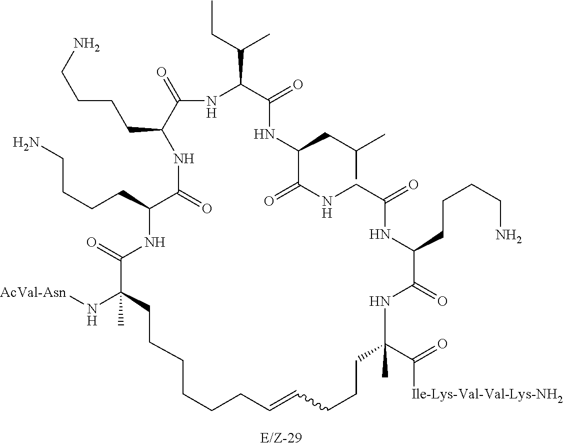

| Entry | Compound | % α-helicity^{a} |
|---|---|---|
| 1 | 21 | 20.8 |
| 2 | E-27 | 80.9 |
| 3 | Z-27 | 71.0 |
| 4 | 23 | 7.5 |
| 5 | E-29 | 21.2 |
| 6 | Z-29 | 23.1 |

^{a}Determined by circular dichroism

It is to be understood that while the invention has been described in conjunction with specific embodiments thereof, that the description above as well as the examples that follow are intended to illustrate and not limit the scope of the invention. Other aspects, advantages, and modifications within the scope of the invention will be apparent to those skilled in the art to which the invention pertains.

### EXPERIMENTAL

In the following examples, efforts have been made to ensure accuracy with respect to numbers used (e.g., amounts, temperature, etc.) but some experimental error and deviation should be accounted for. Unless indicated otherwise, temperature is in degrees C. and pressure is at or near atmospheric. The examples are to be considered as not being limiting of the invention as described herein and are instead provided as representative examples of the catalyst compounds of the invention, the methods that may be used in their preparation, and the methods of using the inventive catalysts.

All reactions were carried out in dry glassware under an atmosphere of argon using standard Schlenk line techniques. Cyclometalated ruthenium catalysts Ru-1 and Ru-2 were obtained from Materia, Inc. and used as received. All solvents were purified by passage through solvent purification columns and further degassed by bubbling argon. Commercially available reagents were used as received unless otherwise noted. Solid substrates were used after purification by column chromatography (SiO₂; (230-400 mesh)). Thin-layer chromatography utilized EMD Sciences silica gel 60 F254 pre-cast glass plates (Cat. No. 1.05714.0001). Microwave-assisted chemistry utilized a Biotage Initiator 2.5 reactor. Wang resin, MBHA resin, and TentaGel MB RAM resin were purchased from Novabiochem or RAPP Polymere. All Boc-protected or Fmoc-protected amino acids were purchased from Chemlmpex or Peptides International. Fmoc-(S)-2-(4-pentenyl)alanine or Fmocl)-2-(7-octenyl)alanine were synthesized as previously described (see Bird, G. H.; Crannell, W. C.; Walensky, L. D. Curr. Protoc. Chem. Biol. 2011, 3, 99) and confirmed by spectroscopic analysis (NMR). HBTU (N,N,N′,N′-tetramethyl-O-(1H-benzotriazol-1-yl)uranium hexafluorophosphate), HATU (1-[Bis(dimethylamino)methylene]-1H-1,2,3,-triazolo[4,5-b]pyridinium 3-oxid hexafluorophosphate), and HOBt (1-hydroxybenzotriazole) were purchased from NovaBioChem. Piperidine, trifluoroacetic acid (TFA), triisopropylsilane (TIPS), and N,N′-dimethylformamide (DMF) were purchased from Sigma-Aldrich. Triethylamine (TEA) or N,N-diisopropylethylamine (DIEA) were purchased from Sigma-Aldrich and distilled prior to use.

Standard NMR spectroscopy experiments were conducted on a Varian INOVA 500 (¹H: 500 MHz, ¹³C: 125 MHz) or Varian INOVA 300 (¹H: 300 MHz, ¹³C: 75 MHz) spectrometer. NMR spectra are reported as 6 values in ppm relative to the reported solvent (CDCl₃ referenced to 7.27, CD₃OD referenced to 3.31). Splitting patterns are abbreviated as follows: singlet (s), doublet (d), triplet (t), quartet (q), multiplet (m), broad (b), apparent (app), and combinations thereof. Spectra were analyzed and processed using MestReNova.

High-resolution mass spectra (HRMS) data was obtained on a JEOL JMS-600H high resolution mass spectrometer operating in FAB⁺ or positive-ion ESI mode. MALDI-TOF spectra were recorded on a Voyager DE-PRO MALDI TOF-MS spectrometer (Applied Biosystems) operating in reflector ion mode using α-cyano-4-hydroxycinnamic acid as the matrix.

Analytical HPLC was performed on an Agilent 1200 Series TOF with an Agilent G1978A Multimode source in electrospray ionization (ESI), or mixed (MM) ionization mode equipped with an Eclipse Plus C₈ column (1.8 μm, 2.1×50 mm). Preparative HPLC was performed with an Agilent 1100 Series HPLC utilizing an Agilent Eclipse XDB-C₁₈ column (5 μm, 9.4×250 mm) or an Agilent Zorbax RX-SIL column (5 μm, 9.4×250 mm) using a gradient of double distilled water and HPLC grade acetonitrile containing 0.1% TFA or 0.1% acetic acid (AcOH). LCMS was performed on an Agilent 1200 Series LCMS equipped with a Quadrupole 6120 MS detector and an Eclipse XDB-C₁₈ reverse-phase column (5, 4.6 μm×150 mm).

The following abbreviations are used in the invention and the examples:
RT room temperature
EtOH ethanol
tBuOH/HOBut tert-butanol
mL milliliter
μL microliter
DMF dimethylformamide
H₂O water
MeNO₂ nitromethane
MgCl₂ magnesium chloride
LiCl lithium chloride
Pbf 2,2,4,6,7-pentamethyldihydrobenzofurane
CDCl₃ deuterated chloroform
THF tetrahydrofuran
HCl hydrochloric acid
Et₂O diethyether
° C. degrees Celsius
h hour
NMP N-methyl-2-pyrrolidone
DCM/CH₂Cl₂ dichloromethane
DCE diethylchloromethane
MeCN acetonitrile
SiO₂ silicagel
EtOAc ethylacetate
MeOH methanol
CD₃OD deutertated methanol
dmso-d⁶ deuterated dimethylsulfoxide
DMSO dimethylsulfoxide
HBTU 2-(1H-benzotriazol-1-yl)-1,1,3,3-tetramethyl uronium hexafluorophosphate
DIEA N,N-diisopropylethylamine
Na₂SO₄ sodium sulfate
Na₂S₂O₃ sodium thiosulfate
TLC thin layer chromatography
R_{f} retention factor
NaHCO₃ sodium bicarbonate
MgSO₄ magnesium sulfate
Ar (g) argon (gas)

### General Procedure for Homoallyl Modification of Peptides

### tert-Butyl (S)-(1-(but-3-en-1-ylamino)-1-oxopropan-2-yl)carbamate (3)

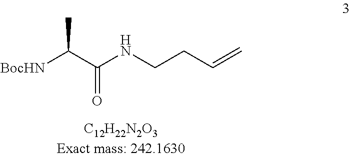 [SMILES: C=CCCNC(=O)[C@H](C)NC(=O)OC(C)(C)C]

A round-bottom flask was charged with Boc-Ala-OH (1.0 g, 5.3 mmol), HOBt (0.72 g, 5.3 mmol, 1.0 eq) and HBTU (3.0 g, 7.9 mmol, 1.5 eq) under Ar(g). To this was added anhydrous DMF (5 mL) and DIEA (2.7 mL, 15.8 mmol, 3 eq.). The reaction mixture was allowed to stir at room temperature for 15 min upon which the solution turned to a pale yellow. A solution of 3-butenylamineHCl (0.85 g, 7.9 mmol, 1.5 eq) in DMF (2 mL) was added and the reaction mixture heated to 50° C. and allowed to stir for 1 h. The solution was cooled to room temperature and H₂O (20 mL) was added, followed by CH₂C₁₂ (50 mL). The organic layer was removed and the aqueous layer was extracted with CH₂C₁₂ (5×50 mL). The combined organic layers were washed with brine (5×50 mL), and dried over Na₂SO₄. The solvent was removed in vacuo and the crude residue was purified by flash chromatography (SiO₂, 0% to 50% EtOAc in hexanes) to provide 1.16 g (91%) of 3 as a white solid: ¹H NMR (300 MHz, CDCl₃) δ 6.51 (bs, 1H), 5.72 (ddt, J=17.1, 10.2, 6.8 Hz, 1H), 5.22 (d, J=7.7 Hz, 1H), 5.12-4.96 (m, 2H), 4.12 (q, J=7.6 Hz, 1H), 3.38-3.18 (m, 2H), 2.22 (qt, J=6.9, 1.3 Hz, 2H), 1.40 (s, 9H), 1.31 (d, J=7.0 Hz, 3H); ¹³C NMR (126 MHz, CDCl₃) δ 172.71, 155.48, 135.04, 117.09, 79.83, 50.02, 38.41, 33.67, 28.30 (3C), 18.64. HRMS (ESI) m/z calcd for C₁₂H₂₂N₂O₃ [M+H]⁺: 243.1630. found 243.1626.

### tert-Butyl (S)-(1-(but-3-en-1-ylamino)-3-methyl-1-oxobutan-2-yl)carbamate (5a)

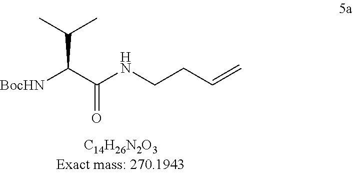 [SMILES: C=CCCNC(=O)[C@@H](NC(=O)OC(C)(C)C)C(C)C]

Following the general procedure for the synthesis of (3), (5a) was synthesized from Boc-Val-OH (1.1 g, 5.3 mmol) in the presence of a stock solution of HOBt (0.72 g, 5.3 mmol, 1.0 eq.), HBTU (3.0 g, 7.9 mmol, 1.5 eq.), and DIEA (2.7 mL, 15.8 mmol, 3 eq.). A solution of 3-butenylamineHCl (0.85 g, 7.9 mmol, 1.5 eq.) in DMF (2 mL) was added and the reaction heated to 50° C. and stirred for 1 h. The crude product was purified by flash chromatography (SiO₂, 0% to 33% EtOAc in hexanes) to provide 1.17 g (82%) of (5a) as a white solid. ¹H NMR (300 MHz, CDCl₃) δ 6.72-6.53 (m, 1H), 5.69 (ddt, J=17.0, 10.1, 6.8 Hz, 1H), 5.32 (d, J=9.3 Hz, 1H), 5.08-4.93 (m, 2H), 3.86 (dd, J=9.1, 6.8 Hz, 1H), 3.36-3.15 (m, 2H), 2.19 (qt, J=6.9, 1.3 Hz, 2H), 2.06-1.95 (m, 1H), 1.37 (s, 9H), 0.87 (dd, J=8.2, 6.7 Hz, 6H); ¹³C NMR (126 MHz, CDCl₃) δ 171.84, 155.94, 135.10, 116.78, 79.34, 59.95, 38.50, 33.71, 30.99, 28.26 (3C), 19.19, 18.10. HRMS (ESI) m/z calcd for C₁₄H₂₆N₂O₃ [M+H]⁺: 271.1943. found 271.1940.

### tert-Butyl ((2S,3R)-1-(but-3-en-1-ylamino)-3-methyl-1-oxopentan-2-yl)carbamate (5b)

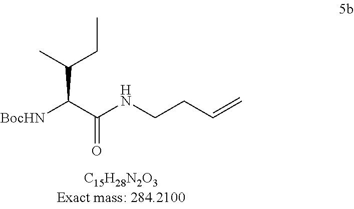 [SMILES: C=CCCNC(=O)[C@@H](NC(=O)OC(C)(C)C)C(C)CC]

Following the general procedure for the synthesis of (3), (5b) was synthesized from Boc-Ile-OH (1.2 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq.) in DMF (2 mL) was added and the reaction heated to 50° C. and stirred for 1 h. The crude product was purified by flash chromatography (SiO₂, 0% to 33% EtOAc in hexanes) to provide 1.27 g (85%) of (5b) as a white solid. ¹H NMR (300 MHz, CDCl₃) δ 6.26 (d, J=6.1 Hz, 1H), 5.73 (ddt, J=17.1, 10.3, 6.8 Hz, 1H), 5.15 (d, J=8.9 Hz, 1H), 5.11-4.98 (m, 2H), 3.88 (dd, J=8.9, 6.7 Hz, 1H), 3.39-3.21 (m, 2H), 2.23 (qt, J=6.8, 1.3 Hz, 2H), 1.90-1.70 (m, 1H), 1.52-1.44 (m, 1H), 1.41 (s, 9H), 1.18-0.98 (m, 1H), 0.95-0.78 (m, 6H); ¹³C NMR (126 MHz, CDCl₃) δ 171.83, 155.87, 135.13, 116.87, 79.43, 59.20, 38.48, 37.15, 33.70, 28.27 (3C), 24.76, 15.43, 11.20. HRMS (ESI) m/z calcd for C₁₅H₂₈N₂O₃ [M+H]⁺: 285.2100. found 284.5101.

### tert-Butyl (S)-(1-(but-3-en-1-ylamino)-4-methyl-1-oxopentan-2-yl)carbamate (5c)

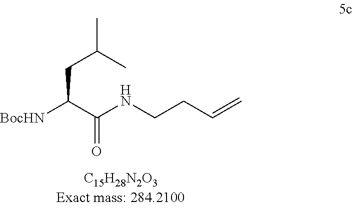 [SMILES: C=CCCNC(=O)[C@H](CC(C)C)NC(=O)OC(C)(C)C]

Following the general procedure for the synthesis of (3), (5c) was synthesized from Boc-Leu-OH (1.3 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.) and 3-butenylamineHCl (0.85 g, 7.9 mmol, 1.5 eq) in DMF at 40° C. The crude product was purified by flash chromatography (SiO₂, 0% to 33% EtOAc in hexanes) to provide 1.18 g (79%) of (5c) as a white solid. ¹H NMR (500 MHz, CDCl₃) δ 6.34 (bs, 1H), 5.74 (ddt, J=17.1, 10.2, 6.8 Hz, 1H), 5.12-5.02 (m, 2H), 5.00 (d, J=8.5 Hz, 1H), 4.06 (q, J=7.6 Hz, 1H), 3.38-3.20 (m, 2H), 2.24 (qt, J=6.8, 1.4 Hz, 2H), 1.66-1.60 (m, 2H), 1.45-1.40 (m, 1H), 1.42 (s, 9H), 0.96-0.86 (m, 6H); ¹³C NMR (126 MHz, CDCl₃) δ 172.83, 155.78, 135.10, 116.81, 79.55, 53.02, 41.51, 38.45, 33.66, 28.27 (3C), 24.65, 22.82, 22.00. HRMS (ESI) m/z calcd for C₁₅H₂₈N₂O₃ [M+H]⁺: 285.2100. found 285.2102.

### tert-Butyl (S)-(1-(but-3-en-1-ylamino)-1-oxo-3-phenylpropan-2-yl)carbamate (5d)

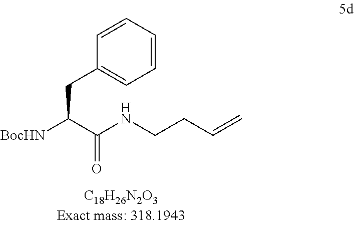 [SMILES: C=CCCNC(=O)[C@H](Cc1ccccc1)NC(=O)OC(C)(C)C]

Following the general procedure for the synthesis of (3), (5d) was synthesized from Boc-Phe-OH (1.4 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq.) in DMF (2 mL) was added and the reaction heated to 50° C. and stirred for 1 h. The crude product was purified by flash chromatography (SiO₂, 0% to 33% EtOAc in hexanes) to provide 1.53 g (91%) of (5d) as a white solid. ¹H NMR (500 MHz, CDCl₃) δ 7.26-7.16 (m, 5H), 6.30 (bs, 1H), 5.63-5.57 (m, 1H), 5.38 (d, J=7.9 Hz, 1H), 5.00-4.90 (m, 2H), 4.41-4.26 (m, 1H), 3.26-3.22 (m, 1H), 3.20-3.13 (m, 1H), 3.04-2.95 (m, 2H), 2.13-2.04 (m, 2H), 1.37 (s, 9H); ¹³C NMR (126 MHz, CDCl₃) δ 171.54, 155.57, 137.05, 135.00 (2C), 129.32 (2C), 128.38, 126.64, 116.85, 79.67, 55.91, 39.02, 38.49, 33.48, 28.28 (3C). HRMS (ESI) m/z calcd for C₁₈H₂₆N₂O₃ [M+H]⁺: 319.1943. found 319.1940.

### tert-Butyl (2-(but-3-en-1-ylamino)-2-oxoethyl)carbamate (5e)

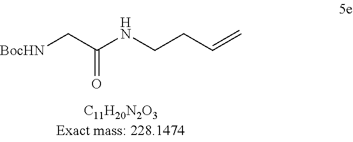 [SMILES: C=CCCNC(=O)CNC(=O)OC(C)(C)C]

Following the general procedure for the synthesis of (3), (5e) was synthesized from Boc-Gly-OH (0.92 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq) in DMF (2 mL) was added and the reaction heated to 50° C. and stirred for 1 h. The crude product was purified by flash chromatography (SiO₂, 3:1 EtOAc:hexane) to provide 0.84 g (70%) of (5e) as a white solid. ¹H NMR (300 MHz, CDCl₃) δ 6.19 (bs, 1H), 5.75 (ddt, J=17.1, 10.3, 6.8 Hz, 1H), 5.13-5.02 (m, 2H), 3.77 (s, 2H), 3.35 (q, J=6.5 Hz, 2H), 2.27 (qt, J=6.8, 1.4 Hz, 2H), 1.45 (s, 9H); ¹³C NMR (126 MHz, CDCl₃) δ 169.85, 156.20, 134.92, 117.10, 80.00, 44.22, 38.45, 33.52, 28.25 (3C). HRMS (ESI) m/z calcd for C₁₁H₂₀N₂O₃ [M+H]⁺: 229.1474. found 229.1476.

### tert-Butyl (S)-2-(but-3-en-1-ylcarbamoyl)pyrrolidine-1-carboxylate (5f)

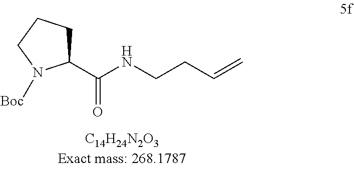 [SMILES: C=CCCNC(=O)[C@@H]1CCCN1C(=O)OC(C)(C)C]

Following the general procedure for the synthesis of (3), (5f) was synthesized from Boc-Pro-OH (1.1 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq.) in DMF (2 mL) was added and the reaction heated to 50° C. and stirred for 1 h. The crude product was purified by flash chromatography (SiO₂, 2:1 EtOAc:hexane) to provide 1.02 g (72%) of (5f) as a white solid (mixture of cis and trans proline isomers). ¹H NMR (500 MHz, CDCl₃) δ 6.84 (bs, 1H), 6.14 (bs, 1H), 5.68-5.61 (m, 1H), 4.99-4.94 (m, 2H), 4.23-3.99 (m, 1H), 3.34-3.19 (m, 4H), 2.14-1.89 (m, 4H), 1.89-1.67 (m, 2H), 1.35 (s, 9H); ¹³C NMR (126 MHz, CDCl₃) δ 172.42, 171.87, 155.57, 154.57, 135.10, 116.88, 80.15, 61.18, 59.90, 46.94, 38.22, 33.66, 31.01, 28.28 (3C), 24.41, 23.56. HRMS (ESI) m/z calcd for C₁₄H₂₄N₂O₃ [M+H]⁺: 269.1787. found 269.1782.

### tert-Butyl (S)-(1-(but-3-en-1-ylamino)-3-(1H-indol-3-yl)-1-oxopropan-2-yl)carbamate (5g)

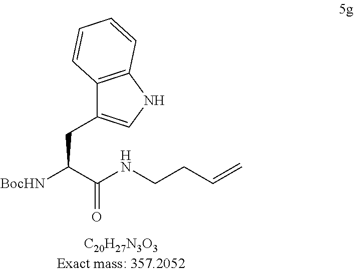 [SMILES: C=CCCNC(=O)[C@H](Cc1c[nH]c2ccccc12)NC(=O)OC(C)(C)C]

Following the general procedure for the synthesis of (3), (5g) was synthesized from Boc-Trp-OH (1.6 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.) A solution of 3-butenylamineHCl (0.85 g, 7.9 mmol, 1.5 eq.) in DMF (2 mL) was added and the reaction heated to 50° C. and stirred for 1 h. The crude product was purified by flash chromatography (SiO₂, 0% to 50% EtOAc in hexanes) to provide 1.40 g (74%) of (5g) as a white solid. ¹H NMR (500 MHz, CDCl₃) δ 8.24 (bs, 1H), 7.66 (d, J=7.9 Hz, 1H), 7.37 (dt, J=8.2, 0.9 Hz, 1H), 7.20 (ddd, J=8.2, 7.0, 1.2 Hz, 1H), 7.13 (ddd, J=8.1, 7.1, 1.1 Hz, 1H), 7.05 (d, J=2.4 Hz, 1H), 5.67 (bs, 1H), 5.58-5.41 (m, 1H), 5.19 (bs, 1H), 4.95-4.75 (m, 2H), 4.39 (q, J=7.2 Hz, 1H), 3.38-3.25 (m, 1H), 3.20-3.11 (m, 3H), 2.05-1.94 (m, 2H), 1.43 (s, 9H); ¹³C NMR (126 MHz, CDCl₃) δ 171.61, 155.48, 136.28, 134.81, 127.41, 123.17, 122.20, 119.65, 118.87, 117.08, 111.26, 110.63, 80.03, 55.34, 38.35, 33.25, 28.65, 28.32 (3C). HRMS (ESI) m/z calcd for C₂₀H₂₇N₃O₃ [M+H]⁺: 358.2052. found 358.2058.

### tert-Butyl (S)-(1-(but-3-en-1-ylamino)-1-oxo-3-(1-tosyl-1H-imidazol-4-yl)propan-2-yl)carbamate (5h)

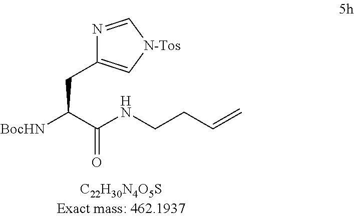

Following the general procedure for the synthesis of (3), (5h) was synthesized from Boc-His(Tos)-OH (2.1 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq.) in DMF (2 mL) was added and the reaction heated to 50° C. and stirred for 1 h. The crude product was purified by flash chromatography (SiO₂, 3:1 EtOAc:hexanes) to provide 1.73 g (71%) of (5h) as a white solid. ¹H NMR (500 MHz, CD₃OD) δ 8.16 (bs, 1H), 7.94-7.86 (m, 2H), 7.46-7.38 (m, 2H), 7.30 (bs, 1H), 5.70 (ddt, J=17.0, 10.2, 6.8 Hz, 1H), 5.02-4.97 (m, 2H), 4.79 (bs, 1H), 4.28 (dd, J=8.9, 5.3 Hz, 1H), 3.20-3.06 (m, 2H), 2.95 (m, 1H), 2.78 (m, 1H), 2.40 (s, 3H), 2.12 (q, J=6.9 Hz, 2H), 1.34 (s, 9H); ¹³C NMR (126 MHz, CD₃OD) δ 172.32, 156.06, 146.73, 140.21, 136.73, 135.07, 134.64, 130.33 (2C), 127.29 (2C), 115.80, 115.09, 79.41, 54.14, 38.34, 33.14, 30.23, 27.24 (3C), 20.34. HRMS (ESI) m/z calcd for C₂₂H₃₀N₄O₅S [M+H]⁺: 462.1937. found 462.1937.

### tert-Butyl (S)-(1-(but-3-en-1-ylamino)-3-(tert-butoxy)-1-oxopropan-2-yl)carbamate (5i)

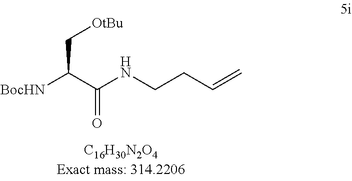 [SMILES: C=CCCNC(=O)[C@H](COC(C)(C)C)NC(=O)OC(C)(C)C]

Following the general procedure for the synthesis of (3), (5i) was synthesized from Boc-Ser(OtBu)-OH (1.4 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq) in DMF (2 mL) was added and the reaction heated to 50° C. and stirred for 1 h. The crude product was purified by flash chromatography (SiO₂, 0% to 33% EtOAc in hexanes) to provide 1.46 g (88%) of (5i) as a white solid. ¹H NMR (500 MHz, CDCl₃) δ 6.61 (s, 1H), 5.76-5.65 (m, 1H), 5.39 (bs, 1H), 5.07-4.99 (m, 2H), 4.1-3.99 (m, 1H), 3.74-3.66 (m, 1H), 3.36-3.24 (m, 3H), 2.24-2.15 (m, 2H), 1.39 (m, 9H), 1.12 (m, 9H); ¹³C NMR (126 MHz, CDCl₃) δ 170.47, 155.42, 135.06, 117.08, 79.74, 73.77, 61.82, 54.24, 38.42, 33.58, 28.25 (3C), 27.37 (3C). HRMS (ESI) m/z calcd for C₁₆H₃₀N₂O₄ [M+H]⁺: 315.2206. found 315.2212.

### tert-Butyl ((2S,3R)-1-(but-3-en-1-ylamino)-3-(tert-butoxy)-1-oxobutan-2-yl)carbamate (5j)

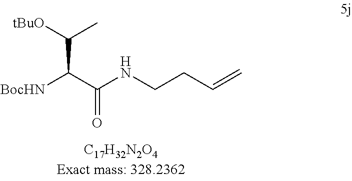 [SMILES: C=CCCNC(=O)[C@@H](NC(=O)OC(C)(C)C)C(C)OC(C)(C)C]

Following the general procedure for the synthesis of (3), (5j) was synthesized from Boc-Thr(OtBu)-OH (1.4 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.) A solution of 3-butenylamineHCl (0.85 g, 7.9 mmol, 1.5 eq.) in DMF (2 mL) was added and the reaction heated to 50° C. and stirred for 1 h. The crude product was purified by flash chromatography (SiO₂, 0% to 25% EtOAc in hexanes) to provide 1.48 g (85%) of (5j) as a white solid. ¹H NMR (500 MHz, CDCl₃) δ 6.87 (t, J=5.4 Hz, 1H), 5.66 (ddt, J=17.1, 10.2, 6.8 Hz, 1H), 5.55 (d, J=5.6 Hz, 1H), 5.05-4.92 (m, 2H), 3.99 (qd, J=6.4, 3.5 Hz, 1H), 3.93 (m, 1H), 3.29-3.19 (m, 2H), 2.15 (qt, J=6.9, 1.2 Hz, 2H), 1.33 (s, 9H), 1.13 (s, 9H), 0.91 (d, J=6.4 Hz, 3H); ¹³C NMR (126 MHz, CDCl₃) δ 169.46, 155.42, 135.02, 117.10, 79.23, 74.92, 66.80, 58.29, 38.35, 33.54, 28.24 (3C), 28.18 (3C), 17.27. HRMS (ESI) m/z calcd for C₁₇H₃₂N₂O₄ [M+H]⁺: 329.2362. found 329.2366.

### tert-Butyl(S)-(1-(but-3-en-1-ylamino)-3-(4-(tert-butoxy)phenyl)-1-oxopropan-2-yl)carbamate (5k)

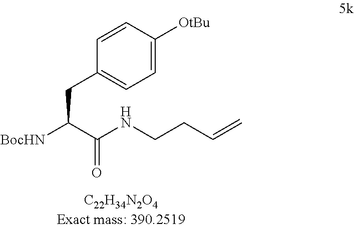 [SMILES: C=CCCNC(=O)[C@H](Cc1ccc(OC(C)(C)C)cc1)NC(=O)OC(C)(C)C]

Following the general procedure for the synthesis of (3), (5k) was synthesized from Boc-Tyr(OtBu)-OH (1.8 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq.) in DMF (2 mL) was added and the reaction heated to 50° C. and stirred for 1 h. The crude product was purified by flash chromatography (SiO₂, 0% to 33% EtOAc in hexanes) to provide 1.73 g (84%) of (5k) as a white solid. ¹H NMR (500 MHz, CDCl₃) δ 7.08 (d, J=8.2 Hz, 2H), 6.91 (d, J=8.4, 2H), 5.81 (bs, 1H), 5.63 (ddt, J=17.1, 10.4, 6.9 Hz, 1H), 5.10 (bs, 1H), 5.04-4.89 (m, 2H), 4.23 (q, J=7.5 Hz, 1H), 3.22 (q, J=6.5 Hz, 2H), 3.03-2.93 (m, 2H), 2.15-2.09 (m, 2H), 1.40 (s, 9H), 1.32 (s, 9H); ¹³C NMR (126 MHz, CDCl₃) δ 171.31, 155.43, 154.15, 134.87, 131.68, 129.69 (2C), 124.15 (2C), 117.06, 79.83, 78.26, 56.00, 38.40, 38.16, 33.47, 28.77 (3C), 28.26 (3C). HRMS (ESI) m/z calcd for C₂₂H₃₄N₂O₄ [M+H]⁺: 391.2519. found 391.2516.

### tert-Butyl (S)-(1-(but-3-en-1-ylamino)-4-(methylthio)-1-oxobutan-2-yl)carbamate (5l)

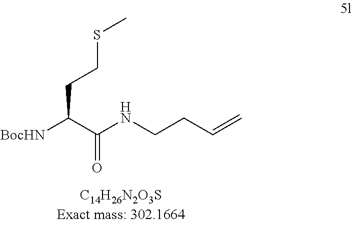 [SMILES: C=CCCNC(=O)[C@H](CCSC)NC(=O)OC(C)(C)C]

Following the general procedure for the synthesis of (3), (5l) was synthesized from Boc-Met-OH (1.3 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq) in DMF (2 mL) was added and the reaction heated to 50° C. and stirred for 1 h. The crude product was purified by flash chromatography (SiO₂, 0% to 33% EtOAc in hexanes) to provide 1.10 g (69%) of (51) as a white solid. ¹H NMR (300 MHz, CDCl₃) δ 6.60 (bs, 1H), 5.71 (ddt, J=17.0, 10.2, 6.8 Hz, 1H), 5.52-5.35 (m, 1H), 5.11-4.95 (m, 2H), 4.29-4.11 (m, 1H), 3.40-3.13 (m, 2H), 2.59-2.38 (m, 2H), 2.21 (qt, J=6.8, 1.3 Hz, 2H), 2.05 (s, 3H), 2.02-1.98 (m, 1H), 1.96-1.77 (m, 1H), 1.39 (s, 9H); ¹³C NMR (126 MHz, CDCl₃) δ 171.72, 155.70, 134.98, 117.01, 79.75, 53.46, 38.51, 33.63, 32.05, 30.10, 28.28 (3C), 15.17. HRMS (ESI) m/z calcd for C₁₄H₂₆N₂O_{3S} [M+H]⁺: 303.1664. found 303.1668.

### tert-Butyl (R)-(1-(but-3-en-1-ylamino)-1-oxo-3-(tritylthio)propan-2-yl)carbamate (5m)

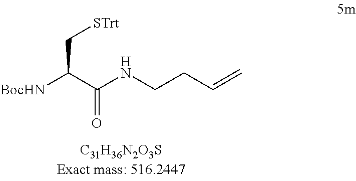

Following the general procedure for the synthesis of (3), (5m) was synthesized from Boc-Cys(Trt)-OH (2.5 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq.) in DMF (2 mL) was added and the reaction heated to 50° C. and stirred for 1 h. The crude product was purified by flash chromatography (SiO₂, 0% to 33% EtOAc in hexanes) to provide 2.04 g (75%) of (5m) as a white solid. ¹H NMR (500 MHz, CDCl₃) δ 7.44-7.40 (m, 6H), 7.32-7.27 (m, 6H), 7.25-7.20 (m, 3H), 6.05 (bs, 1H), 5.71 (ddt, J=17.0, 10.2, 6.8 Hz, 1H), 5.10-4.99 (m, 2H), 4.82 (bs, 1H), 3.87-3.84 (m, 1H), 3.32-3.19 (m, 2H), 2.75-2.71 (m, 1H), 2.54-2.50 (m, 1H), 2.21 (qt, J=6.8, 1.3 Hz, 2H), 1.42 (s, 9H); ¹³C NMR (126 MHz, CDCl₃) δ 170.37, 155.35, 144.47 (3C), 135.01, 129.58 (6C), 128.03 (6C), 126.85 (3C), 117.23, 80.06, 67.13, 53.57, 38.51, 34.05, 33.58, 28.33 (3C). HRMS (ESI) m/z calcd for C₃₁H₃₆N₂O₃S [M+H]⁺: 517.2447. found 517.2450.

### tert-Butyl (S)-4-(but-3-en-1-ylamino)-3-((tert-butoxycarbonyl)amino)-4-oxobutanoate (5n)

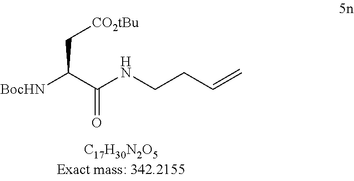 [SMILES: C=CCCNC(=O)[C@H](CC(=O)OC(C)(C)C)NC(=O)OC(C)(C)C]

Following the general procedure for the synthesis of (3), (5n) was synthesized from Boc-Asp(OtBu)-OH (1.5 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq) in DMF (2 mL) was added and the reaction heated to 50° C. and stirred for 1 h. The crude product was purified by flash chromatography (SiO₂, 0% to 50% EtOAc in hexanes) to provide 1.55 g (86%) of (5n) as a white solid. ¹H NMR (500 MHz, CDCl₃) δ 6.58 (t, J=5.7 Hz, 1H), 5.74-5.67 (m, 2H), 5.11-4.96 (m, 2H), 4.46-4.30 (m, 1H), 3.32-3.23 (m, 2H), 2.80 (dd, J=16.8, 4.9 Hz, 1H), 2.56 (dd, J=16.8, 6.6 Hz, 1H), 2.21 (qt, J=6.7, 1.3 Hz, 2H), 1.41 (s, 9H), 1.40 (s, 9H); ¹³C NMR (126 MHz, CDCl₃) δ 171.17, 170.71, 155.45, 134.96, 117.15, 81.49, 80.11, 50.69, 38.50, 37.36, 33.55, 28.27 (3C), 27.99 (3C). HRMS (ESI) m/z calcd for C₁₇H₃₀N₂O₅ [M+H]⁺: 343.2155. found 343.2151.

### tert-Butyl (S)-5-(but-3-en-1-ylamino)-4-((tert-butoxycarbonyl)amino)-5-oxopentanoate (5o)

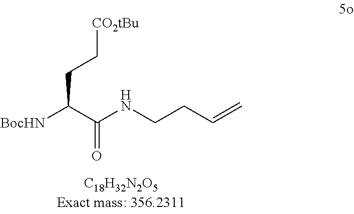 [SMILES: C=CCCNC(=O)[C@H](CCC(=O)OC(C)(C)C)NC(=O)OC(C)(C)C]

Following the general procedure for the synthesis of (3), (5o) was synthesized from Boc-Glu(OtBu)-OH (1.6 g, 5.3 mmol) in the presence of a stock solution of HOBt (0.72 g, 5.3 mmol, 1.0 eq.), HBTU (3.0 g, 7.9 mmol, 1.5 eq.), and DIEA (2.7 mL, 15.8 mmol, 3 eq.). A solution of 3-butenylamineHCl (0.85 g, 7.9 mmol, 1.5 eq.) in DMF (2 mL) was added and the reaction heated to 50° C. and stirred for 1 h. The crude product was purified by flash chromatography (SiO₂, 0% to 40% EtOAc in hexanes) to provide 1.53 g (82%) of (5o) as a white solid. ¹H NMR (500 MHz, CDCl₃) δ 6.93-6.81 (m, 1H), 5.69-5.57 (m, 2H), 4.99-4.87 (m, 2H), 4.07-4.03 (m, 1H), 3.26-3.21 (m, 1H), 3.14-3.10 (m, 1H), 2.29-2.07 (m, 4H), 1.99-1.87 (m, 1H), 1.84-1.71 (m, 1H), 1.31 (s, 18H). ¹³C NMR (126 MHz, CDCl₃) δ 172.32, 171.69, 155.62, 134.98, 116.79, 80.32, 79.48, 53.85, 38.47, 33.57, 31.66, 28.21 (3C), 27.93 (3C), 27.90. HRMS (ESI) m/z calcd for C₁₇H₃₀N₂O₅ [M+H]⁺: 357.2311. found 357.2314.

### tert-Butyl (S)-(1-(but-3-en-1-ylamino)-1,4-dioxo-4-(tritylamino)butan-2-yl)carbamate (5p)

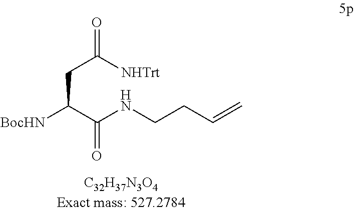

Following the general procedure for the synthesis of (3), (5p) was synthesized from Boc-Asn(Trt)-OH (2.5 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq) in DMF (2 mL) was added and the reaction heated to 50° C. and stirred for 1 h. The crude product was purified by flash chromatography (SiO₂, 0% to 50% EtOAc in hexanes) to provide 2.17 g (78%) of (5p) as a white solid. ¹H NMR (500 MHz, CDCl₃) δ 7.31-7.17 (m, 16H), 6.74 (bs, 1H), 6.29 (bs, 1H), 5.72 (ddt, J=17.1, 10.3, 6.8 Hz, 1H), 5.12-4.99 (m, 2H), 4.43 (dd, J=8.5, 4.6 Hz, 1H), 3.34-3.14 (m, 2H), 3.14-2.99 (m, 1H), 2.54 (dd, J=15.0, 5.8 Hz, 1H), 2.21-2.17 (m, 2H), 1.43 (s, 9H); ¹³C NMR (126 MHz, CDCl₃) δ 171.43, 170.49, 155.68, 144.44 (3C), 134.98, 128.73 (6C), 127.88 (6C), 126.93 (3C), 117.20, 79.94, 70.63, 51.76, 38.54, 38.18, 33.45, 28.40 (3C). HRMS (ESI) m/z calcd for C₃₂H₃₇N₃O₄ [M+H]⁺: 528.2784. found 528.2782.

### tert-Butyl (S)-(1-(but-3-en-1-ylamino)-1,5-dioxo-5-(tritylamino)pentan-2-yl)carbamate (5q)

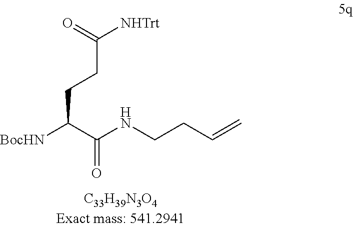

Following the general procedure for the synthesis of (3), (5q) was synthesized from Boc-Gln(Trt)-OH (2.5 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamine-HCl (0.85 g, 7.9 mmol, 1.5 eq.) in DMF (2 mL) was added and the reaction heated to 50° C. and stirred for 1 h. The crude product was purified by flash chromatography (SiO₂, 0% to 50% EtOAc in hexanes) to provide 2.37 g (83%) of (5q) as a white solid. ¹H NMR (300 MHz, CDCl₃) δ 7.37-7.12 (m, 17H), 6.41 (bs, 1H), 5.79-5.56 (m, 2H), 5.12-4.91 (m, 2H), 3.00-3.93 (m, 1H), 3.34-3.04 (m, 2H), 2.56-2.24 (m, 2H), 2.25-2.08 (m, 2H), 2.07-1.76 (m, 2H), 1.43 (s, 9H); ¹³C NMR (126 MHz, CDCl₃) δ 171.86, 171.47, 155.88, 144.59 (3C), 135.05, 128.69 (6C), 127.92 (6C), 126.95 (3C), 117.09, 79.74, 70.57, 53.62, 38.54, 33.73, 33.62, 29.87, 28.36 (3C). HRMS (ESI) m/z calcd for C₃₃H₃₉N₃O₄ [M+H]⁺: 542.2941. found 542.2942.

### di-tert-Butyl (6-(but-3-en-1-ylamino)-6-oxohexane-1,5-diyl)(S)-dicarbamate (5r)

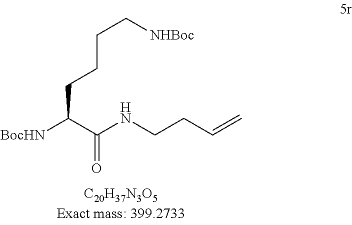 [SMILES: C=CCCNC(=O)[C@H](CCCCNC(=O)OC(C)(C)C)NC(=O)OC(C)(C)C]

Following the general procedure for the synthesis of (3), (5r) was synthesized from Boc-Lys(Boc)-OH (1.8 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq.) in DMF (2 mL) was added and the reaction heated to 50° C. and stirred for 1 h. The crude product was purified by flash chromatography (SiO₂, 0% to 50% EtOAc in hexanes) to provide 1.90 g (90%) of (5r) as a white solid. ¹H NMR (500 MHz, CDCl₃) δ 6.60 (bs, 1H), 5.69 (ddt, J=17.1, 10.2, 6.8 Hz, 1H), 5.43-5.29 (m, 1H), 5.07-4.96 (m, 2H), 4.83-4.70 (m, 1H), 4.02 (m, 1H), 3.35-3.25 (m, 1H), 3.21 (m, 1H), 3.11-2.98 (m, 2H), 2.20 (qt, J=6.8, 1.3 Hz, 2H), 1.79-1.69 (m, 1H), 1.62-1.51 (m, 1H), 1.49-1.41 (m, 2H), 1.41-1.34 (bs, 18H), 1.35-1.26 (m, 2H); ¹³C NMR (126 MHz, CDCl₃) δ 172.14, 156.13, 155.76, 135.05, 117.03, 79.75, 78.96, 54.36, 39.92, 38.44, 33.63, 32.15, 29.59, 28.39 (3C), 28.30 (3C), 22.60. HRMS (ESI) m/z calcd for C₂₀H₃₇N₃O₅ [M+H]⁺: 400.2733. found 400.2730.

### tert-Butyl (S)-(1-(but-3-en-1-ylamino)-1-oxo-5-(3-((2,2,4,6,7-pentamethyl-2,3-dihydrobenzofuran-5-yl)sulfonyl)guanidino)pentan-2-yl)carbamate (5s)

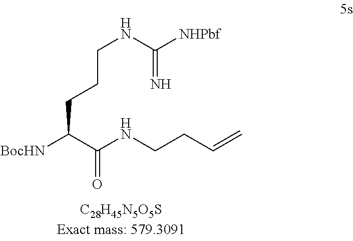

Following the general procedure for the synthesis of (3), (5s) was synthesized from Boc-Arg(Pbf)-OH (2.8 g, 5.3 mmol) in the presence of a stock solution of HOBt (0.72 g, 5.3 mmol, 1.0 eq.), HBTU (3.0 g, 7.9 mmol, 1.5 eq.), DIEA (2.7 mL, 15.8 mmol, 3 eq.). A solution of 3-butenylamineHCl (0.85 g, 7.9 mmol, 1.5 eq) in DMF (2 mL) was added and the reaction heated to 50° C. and stirred for 1 h. The crude product was purified by flash chromatography (SiO₂, EtOAc) to provide 2.14 g (70%) of 5s as a white solid. ¹H NMR (500 MHz, CDCl₃) δ 7.03 (bs, 1H), 6.36 (bs, 2H), 5.79-5.60 (m, 2H), 5.09-4.96 (m, 2H), 4.13 (m, 1H), 3.36-3.17 (m, 4H), 2.96 (s, 2H), 2.58 (s, 3H), 2.51 (s, 3H), 2.23 (q, J=6.9 Hz, 2H), 2.10 (s, 3H), 1.80-1.78 (m, 1H), 1.69-1.53 (m, 4H), 1.47 (s, 6H), 1.40 (s, 9H); ¹³C NMR (126 MHz, CDCl₃) δ 172.36, 158.82, 156.44, 156.00, 138.31, 135.13, 132.71, 132.24, 124.65, 117.55, 116.85, 86.40, 79.92, 64.33, 53.99, 43.25, 40.55, 38.71, 33.56, 30.43, 28.57 (2C), 28.33 (3C), 25.56, 19.26, 17.92, 12.43. HRMS (ESI) m/z calcd for C₂₈H₄₅N₅O₆S [M+H]⁺: 580.3091. found 580.3096.

### General Procedure for Homodimerization of Amino Acids

### di-tert-Butyl ((2S,2′S)-(hex-3-ene-1,6-diylbis(azanediyl))bis(1-oxopropane-1,2-diyl))dicarbamate (4)

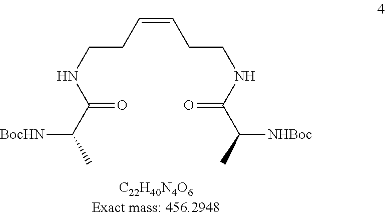 [SMILES: C[C@H](NC(=O)OC(C)(C)C)C(=O)NCC/C=C\CCNC(=O)[C@H](C)NC(=O)OC(C)(C)C]

The homoallyl-modified alanine (3) (0.20 g, 0.83 mmol) was dissolved in THF (1.4 mL) under a gentle stream of argon. A solution of catalyst Ru-1 or Ru-2 (619 μl of a 0.10 M solution in THF) was added and the reaction heated to 40° C. and stirred for 4 h. The solution was allowed to cool to room temperature upon which an excess of ethyl vinyl ether (1.0 mL, 10.4 mmol, 12 eq.) was added to quench the reaction. The solvent was removed in vacuo and the residue purified by column chromatography (SiO₂; 0% to 25% EtOAc in hexane) to afford 0.28 g (74%) of product (4) as a white solid. ¹H NMR (500 MHz, CDCl₃) δ 7.16 (bs, 2H), 5.49-5.34 (m, 2H), 5.24 (d, J=8.4 Hz, 2H), 4.31-4.14 (m, 2H), 3.73-3.60 (m, 2H), 3.03-2.86 (m, 2H), 2.26-2.16 (m, 4H), 1.43 (s, 18H), 1.39-1.27 (m, 6H); ¹³C NMR (126 MHz, CDCl₃) δ 173.34 (2C), 155.73 (2C), 129.23 (2C), 79.91 (2C), 50.08 (2C), 38.65 (2C), 28.35 (3C), 28.32 (3C), 27.97 (2C), 18.53 (2C). HRMS (ESI) m/z calcd for C₂₂H₄₀N₄O₆ [M+H]⁺: 457.2948. found 457.2945.

### di-tert-Butyl ((2S,2′S)-(hex-3-ene-1,6-diylbis(azanediyl))bis(3-methyl-1-oxobutane-1,2-diyl))dicarbamate (6a)

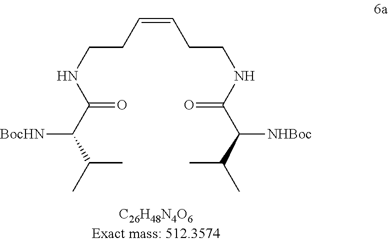 [SMILES: CC(C)[C@H](NC(=O)OC(C)(C)C)C(=O)NCC/C=C\CCNC(=O)[C@@H](NC(=O)OC(C)(C)C)C(C)C]

Following the procedure for (4), the homodimerization product (6a) was obtained when homoallyl-modified valine (5a) (0.22 g, 0.81 mmol) was reacted with catalyst Ru-1 or Ru-2 (610 μl of a 0.10 M solution in THF) in THF (1.4 mL) under argon for 4 h and quenched with excess ethyl vinyl ether. The residue was purified by column chromatography (SiO₂; 0% to 50% EtOAc in hexane) to afford 0.30 g (71%) of the product (6a) as a white solid. ¹H NMR (500 MHz, CDCl₃) δ 7.43-7.36 (m, 2H), 5.42-5.34 (m, 2H), 5.17 (d, J=9.6 Hz, 2H), 3.95 (dd, J=9.7, 7.7 Hz, 2H), 3.93-3.83 (m, 2H), 2.80-2.76 (m, 2H), 2.25-2.21 (m, 2H), 2.18-2.12 (m, 2H), 1.94-1.90 (m, 2H), 1.67 (bs, 2H), 1.43 (s, 18H), 0.96 (d, J=6.7 Hz, 6H), 0.95 (d, J=6.6 Hz, 6H); ¹³C NMR (126 MHz, CDCl₃) δ 172.31 (2C), 156.28 (2C), 129.56 (2C), 79.70 (2C), 60.43 (2C), 38.07 (2C), 30.92 (2C), 28.32 (6C), 19.21 (2C), 18.59 (2C). HRMS (ESI) m/z calcd for C₂₆H₄₈N₄O₆ [M+H]⁺: 513.3574. found 513.3570.

### di-tert-Butyl ((2S,2S,3R,3′R)-(hex-3-ene-1,6-diylbis(azanediyl))bis(3-methyl-1-oxopentan-1,2-diyl))dicarbamate (6b)

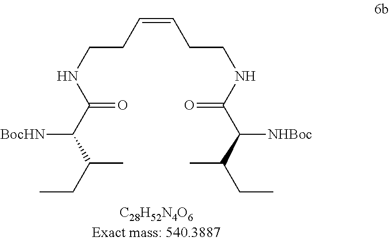 [SMILES: CCC(C)[C@H](NC(=O)OC(C)(C)C)C(=O)NCC/C=C\CCNC(=O)[C@@H](NC(=O)OC(C)(C)C)C(C)CC]

Following the procedure for (4), the homodimerization product (6b) was obtained when homoallyl-modified isoleucine (5b) (0.21 g, 0.74 mmol) was reacted with catalyst Ru-1 or Ru-2 (553 μl of a 0.10 M solution in THF) in THF (1.3 mL) under argon. The residue was purified by column chromatography (SiO₂; 0% to 50% EtOAc in hexane) to afford 0.27 g (68%) of the product (6b) as a white solid. ¹H NMR (500 MHz, CDCl₃) δ 7.52-7.45 (m, 2H), 5.42-5.33 (m, 2H), 5.14 (d, J=9.7 Hz, 2H), 4.02-3.93 (m, 4H), 2.81-2.70 (m, 2H), 2.27-2.18 (m, 2H), 2.18-2.08 (m, 2H), 1.76-1.63 (m, 2H), 1.58 (m, 2H), 1.42 (s, 18H), 1.21-1.09 (m, 2H), 0.94-0.82 (m, 12H); ¹³C NMR (126 MHz, CDCl₃) δ 172.46 (2C), 156.14 (2C), 129.59 (2C), 79.64 (2C), 59.02 (2C), 37.95 (2C), 36.86 (2C), 29.69 (2C), 28.33 (6C), 24.90 (2C), 15.31 (2C), 10.60 (2C). HRMS (ESI) m/z calcd for C₂₈H₅₂N₄O₆ [M+H]⁺: 541.3887. found 541.3880.

### di-tert-Butyl ((2S,2′S)-(hex-3-ene-1,6-diylbis(azanediyl))bis(4-methyl-1-oxopentane-1,2-diyl))dicarbamate (6c)

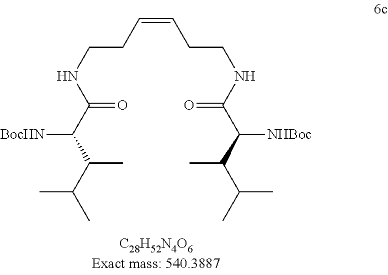 [SMILES: CC(C)C(C)[C@H](NC(=O)OC(C)(C)C)C(=O)NCC/C=C\CCNC(=O)[C@@H](NC(=O)OC(C)(C)C)C(C)C(C)C]

Following the procedure for (4), the homodimerization product (6c) was obtained when homoallyl-modified leucine (5c) (0.18 g, 0.63 mmol) was reacted with catalyst Ru-1 or Ru-2 (475 μl of a 0.10 M solution in THF) in THF (1.1 mL) under argon. The residue was purified by column chromatography (SiO₂; 0% to 50% EtOAc in hexane) to afford 0.24 g (70%) of the product (6c) as a white solid. ¹H NMR (500 MHz, CDCl₃) δ 7.64-7.55 (m, 2H), 5.40-5.38 (m, 2H), 5.06 (d, J=9.1 Hz, 2H), 4.28-4.23 (m, 2H), 3.92-3.90 (m, 2H), 2.77-2.73 (m, 2H), 2.23 (m, 2H), 2.20-2.11 (m, 2H), 1.70-1.64 (m, 4H), 1.56-1.47 (m, 4H), 1.42 (s, 18H), 0.92 (d, J=6.6 Hz, 6H), 0.88 (d, J=6.6 Hz, 6H); ¹³C NMR (126 MHz, CDCl₃) δ 173.36 (2C), 155.95 (2C), 129.57 (2C), 79.75 (2C), 53.17 (2C), 41.68 (2C), 38.30 (2C), 28.33 (6C), 24.67 (4C), 23.04 (2C), 21.82 (2C). HRMS (ESI) m/z calcd for C₂₈H₅₂N₄O₆ [M+H]⁺: 541.3887. found 541.3879.

### di-tert-Butyl ((2S,2′S)-(hex-3-ene-1,6-diylbis(azanediyl))bis(1-oxo-3-phenylpropane-1,2-diyl))dicarbamate (6d)

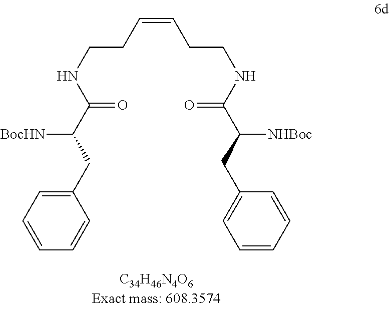 [SMILES: CC(C)(C)OC(=O)N[C@@H](Cc1ccccc1)C(=O)NCC/C=C\CCNC(=O)[C@H](Cc1ccccc1)NC(=O)OC(C)(C)C]

Following the procedure for (4), the homodimerization product (6d) was obtained when homoallyl-modified phenylalanine (5d) (0.23 g, 0.72 mmol) was reacted with catalyst Ru-1 or Ru-2 (542 μl of a 0.10 M solution in THF) in THF (1.2 mL) under argon. The residue was purified by column chromatography (SiO₂; 0% to 50% EtOAc in hexane) to afford 0.32 g (73%) of the product (6d) as a white solid. ¹H NMR (300 MHz, CDCl₃) δ 7.32-7.09 (m, 10H), 7.10-6.91 (m, 2H), 5.36-5.33 (m, 4H), 5.29 (bs, 2H), 4.50-4.30 (m, 2H), 3.62 (m, 2H), 3.12-2.72 (m, 4H), 2.15-2.04 (m, 4H), 1.35 (s, 18H); ¹³C NMR (126 MHz, CDCl₃) δ 171.98 (2C), 155.80 (2C), 137.01 (2C), 129.21 (4C), 128.46 (4C), 126.63 (2C), 79.94 (2C), 56.03 (2C), 38.92 (2C), 38.61 (2C), 28.28 (6C), 27.91 (2C). HRMS (ESI) m/z calcd for C₃₄H₄₈N₄O₆ [M+H]⁺: 609.3574. found 609.3578.

### di-tert-Butyl ((2S,2′S)-(hex-3-ene-1,6-diylbis(azanediyl))bis(3-(1H-indol-3-yl)-1-oxopropane-1,2-diyl))dicarbamate (6g)

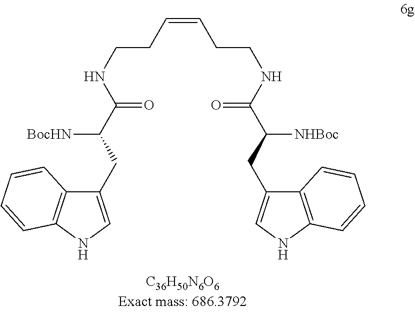 [SMILES: CC(C)(C)OC(=O)N[C@@H](Cc1c[nH]c2ccccc12)C(=O)NCC/C=C\CCNC(=O)[C@H](Cc1c[nH]c2ccccc12)NC(=O)OC(C)(C)C]

Following the procedure for (4), the homodimerization product (6g) was obtained when homoallyl-modified tryptophan (5g) (0.21 g, 0.59 mmol) was reacted with catalyst Ru-1 or Ru-2 (440 μl of a 0.10 M solution in THF) in THF (1.0 mL) under argon. The residue was purified by column chromatography (SiO₂; 3:1 EtOAc:hexanes) to afford 0.26 g (66%) of the product (6g) as a white solid. ¹H NMR (500 MHz, CDCl₃) δ 8.74 (bs, 2H), 7.59 (d, J=7.9 Hz, 2H), 7.34 (d, J=8.1 Hz, 2H), 7.16 (ddd, J=8.1, 6.9, 1.1 Hz, 2H), 7.06 (t, J=7.5 Hz, 2H), 6.94 (bs, 2H), 6.37 (bs, 2H), 5.38 (d, J=8.1 Hz, 2H), 5.14-5.12 (m, 2H), 4.56-4.35 (m, 2H), 3.30-3.11 (m, 6H), 2.98-2.96 (m, 2H), 1.91-1.71 (m, 4H), 1.42 (s, 18H); ¹³C NMR (126 MHz, CDCl₃) δ 172.14 (2C), 155.71 (2C), 136.28 (2C), 128.53 (2C), 127.43 (2C), 123.38 (2C), 121.97 (2C), 119.47 (2C), 118.74 (2C), 111.34 (2C), 110.39 (2C), 80.08 (2C), 55.39 (2C), 38.77 (2C), 29.71 (2C), 28.34 (6C), 27.30 (2C). HRMS (ESI) m/z calcd for C₃₈H₅₀N₆O₆ [M+H]⁺: 687.3792. found 687.3795.

### di-tert-Butyl ((5S,16S)-2,2,19,19-tetramethyl-6,15-dioxo-3,18-dioxa-7,14-diazaicos-10-ene-5, 16-diyl)dicarbamate (6i)

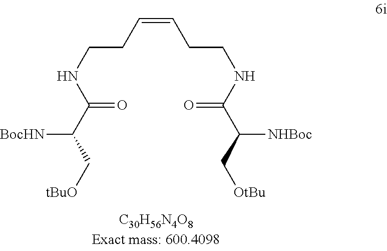 [SMILES: CC(C)(C)OC[C@H](NC(=O)OC(C)(C)C)C(=O)NCC/C=C\CCNC(=O)[C@H](COC(C)(C)C)NC(=O)OC(C)(C)C]

Following the procedure for (4), the homodimerization product (6i) was obtained when homoallyl-modified serine (5i) (0.20 g, 0.63 mmol) was reacted with catalyst Ru-1 or Ru-2 (477 μl of a 0.10 M solution in THF) in THF (1.1 mL) under argon. The residue was purified by column chromatography (SiO₂; 0% to 50% EtOAc in hexane) to afford 0.27 g (72%) of the product (6i) as a white solid. ¹H NMR (500 MHz, CDCl₃) δ 6.70 (s, 2H), 5.48-5.43 (m, 4H), 4.12 (bs, 2H), 3.76-3.74 (m, 2H), 3.36-3.33 (m, 4H), 3.27-3.24 (m, 2H), 2.32-2.20 (m, 4H), 1.45 (s, 18H), 1.17 (s, 18H); ¹³C NMR (126 MHz, CDCl₃) δ 170.66 (2C), 155.56 (2C), 128.55 (2C), 79.90 (2C), 73.86 (2C), 61.88 (2C), 54.34 (2C), 39.09 (2C), 28.32 (6C), 27.48 (2C), 27.44 (6C). HRMS (ESI) m/z calcd for C₃₀H₅₆N₄O₈ [M+H]⁺: 601.4098. found 601.4100.

### di-tert-Butyl((4R,5S,16R,17S)-2,2,4,17,19,19-hexamethyl-6,15-dioxo-3,18-dioxa-7,14-diazaicos-10-ene-5,16-diyl)dicarbamate (6j)

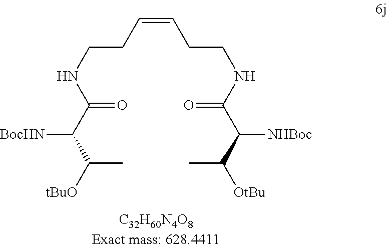 [SMILES: CC(OC(C)(C)C)[C@H](NC(=O)OC(C)(C)C)C(=O)NCC/C=C\CCNC(=O)[C@@H](NC(=O)OC(C)(C)C)C(C)OC(C)(C)C]

Following the procedure for (4), the homodimerization product (6j) was obtained when homoallyl-modified threonine (5j) (0.19 g, 0.58 mmol) was reacted with catalyst Ru-1 or Ru-2 (433 μl of a 0.10 M solution in THF) in THF (1.0 mL) under argon. The residue was purified by column chromatography (SiO₂; 0% to 40% EtOAc in hexane) to afford 0.26 g (73%) of the product (6j) as a white solid. ¹H NMR (500 MHz, CDCl₃) δ 6.99 (t, J=5.4 Hz, 2H), 5.65 (d, J=5.8 Hz, 2H), 5.50 (td, J=4.4, 2.1 Hz, 2H), 4.11 (qd, J=6.3, 3.3 Hz, 2H), 4.08-4.00 (m, 2H), 3.40-3.23 (m, 4H), 2.31-2.25 (m, 4H), 1.45 (s, 18H), 1.25 (s, 18H), 1.03 (d, J=6.3 Hz, 6H); ¹³C NMR (126 MHz, CDCl₃) δ 169.73 (2C), 155.61 (2C), 128.63 (2C), 79.53 (2C), 75.13 (2C), 66.92 (2C), 58.39 (2C), 39.05 (2C), 28.37 (6C), 28.31 (6C), 27.55 (2C), 17.41 (2C). HRMS (ESI) m/z calcd for C₃₂H₆₀N₄O₈ [M+H]⁺: 629.4411. found 629.4413.

### di-tert-Buty′((2S,2′S)-(hex-3-ene-1,6-diylbis(azanediyl))bis(3-(4-(tert-butoxy)phenyl)-1-oxopropane-1,2-diyl))dicarbamate (6k)

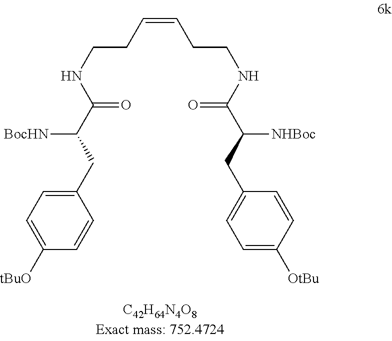 [SMILES: CC(C)(C)OC(=O)N[C@@H](Cc1ccc(OC(C)(C)C)cc1)C(=O)NCC/C=C\CCNC(=O)[C@H](Cc1ccc(OC(C)(C)C)cc1)NC(=O)OC(C)(C)C]

Following the procedure for (4), the homodimerization product (6k) was obtained when homoallyl-modified tyrosine (5k) (0.17 g, 0.44 mmol) was reacted with catalyst Ru-1 or Ru-2 (326 μl of a 0.10 M solution in THF) in THF (0.76 mL) under argon. The residue was purified by column chromatography (SiO₂; 0% to 50% EtOAc in hexane) to afford 0.21 g (64%) of the product (6k) as a white solid. ¹H NMR (300 MHz, CDCl₃) δ 7.11-7.03 (m, 4H), 6.91-6.85 (m, 4H), 5.34 (dt, J=6.4, 4.8 Hz, 2H), 5.23 (d, J=8.8 Hz, 2H), 4.42-4.35 (m, 2H), 3.59-3.51 (m, 2H), 2.97-2.83 (m, 6H), 2.17-2.05 (m, 6H), 1.36 (s, 18H), 1.31 (s, 18H); ¹³C NMR (126 MHz, CDCl₃) δ 171.84 (2C), 155.70 (2C), 154.04 (2C), 131.82 (2C), 129.67 (4C), 129.02 (2C), 124.23 (4C), 79.90 (2C), 78.32 (2C), 55.98 (2C), 38.61 (2C), 38.21 (2C), 28.82 (6C), 28.28 (6C), 27.80 (2C). HRMS (ESI) m/z calcd for C₄₂H₆₄N₄O₈ [M+H]⁺: 753.4724. found 753.4719.

### di-tert-Butyl((4R,15R)-5,14-dioxo-1,1,1,18,18,18-hexaphenyl-2,17-dithia-6,13-diazaoctadec-9-ene-4,15-diyl)dicarbamate (6m)

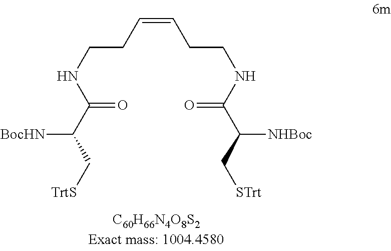

Following the procedure for (4), the homodimerization product (6m) was obtained when homoallyl-modified cysteine (5m) (0.23 g, 0.44 mmol) was reacted with catalyst Ru-1 or Ru-2 (334 μl of a 0.10 M solution in THF) in THF (0.78 mL) under Ar(g). The residue was purified by column chromatography (SiO₂; 0% to 40% EtOAc in hexane) to afford 0.25 g (55%) of the product (6m) as a white solid. ¹H NMR (500 MHz, CDCl₃) δ 7.41 (m, 12H), 7.31-7.24 (m, 12H), 7.23-7.17 (m, 6H), 6.72 (bs, 1H), 6.11 (bs, 1H), 5.38-5.34 (m, 2H), 5.00-4.86 (m, 2H), 4.06-4.02 (m, 1H), 3.86 (bs, 1H), 3.51-3.48 (m, 1H), 3.19-3.16 (m, 1H), 2.98-2.92 (m, 1H), 2.67 (bs, 1H), 2.53 (m, 4H), 2.23-2.10 (m, 4H), 1.42-1.37 (m, 18H); ¹³C NMR (126 MHz, CDCl₃) δ 170.67 (2C), 155.52 (2C), 146.86 (2C), 144.46 (3C), 144.43 (3C), 129.57 (2C), 129.56 (6C), 128.92 (2C), 128.02 (2C), 128.00 (6C), 127.95 (2C), 127.91 (2C), 127.23 (2C), 126.84 (2C), 126.77 (2C), 80.14 (2C), 66.95 (2C), 53.58 (2C), 38.82 (2C), 33.98 (2C), 29.69 (2C), 28.32 (3C), 28.29 (3C), 27.66 (2C). HRMS (ESI) m/z calcd for C₆₀H₆₈N₄O₆S₂ [M+H]⁺: 1006.35. found 1006.44.

### tert-Butyl(6S,17S)-6-(2-(tert-butoxy)-2-oxoethyl)-17-((tert-butoxycarbonyl)amino)-2,2-dimethyl-4,7,16-trioxo-3-oxa-5,8,15-triazanonadec-11-en-19-oate (6n)

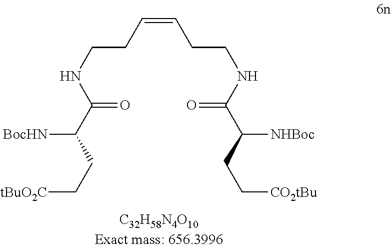 [SMILES: CC(C)(C)OC(=O)CC[C@H](NC(=O)OC(C)(C)C)C(=O)NCC/C=C\CCNC(=O)[C@H](CCC(=O)OC(C)(C)C)NC(=O)OC(C)(C)C]

Following the procedure for (4), the homodimerization product (6n) was obtained when homoallyl-modified aspartate (5n) (0.19 g, 0.55 mmol) was reacted with catalyst Ru-1 or Ru-2 (416 μl of a 0.10 M solution in THF) in THF (1.0 mL) under Ar(g). The residue was purified by column chromatography (SiO₂; 0% to 50% EtOAc in hexane) to afford 0.22 g (61%) of the product (6n) as a white solid. ¹H NMR (500 MHz, CDCl₃) δ 6.74 (bs, 2H), 5.76 (bs, 2H), 5.48-5.38 (m, 2H), 4.47-4.42 (m, 2H), 3.41-3.35 (m, 2H), 3.31-3.29 (m, 2H), 3.23-3.18 (m, 2H), 2.84-2.80 (m, 2H), 2.67-2.57 (m, 2H), 2.26-2.24 (m, 4H), 1.45 (s, 18H), 1.44 (s, 18H); ¹³C NMR (126 MHz, CDCl₃) δ 170.96 (2C), 155.54 (2C), 128.59 (2C), 81.51 (2C), 80.17 (2C), 50.82 (2C), 39.12 (2C), 37.48 (2C), 29.69 (2C), 28.33 (6C), 28.03 (6C), 27.41 (2C). HRMS (ESI) m/z calcd for C₃₂H₅₆N₄O₁₀ [M+H]⁺: 657.8180. found 657.8177.

### tert-Butyl(6S,17S)-6-(3-(tert-butoxy)-3-oxopropyl)-17-((tert-butoxycarbonyl)amino)-2,2-dimethyl-4,7,16-trioxo-3-oxa-5,8,15-triazaicos-11-en-20-oate (6o)

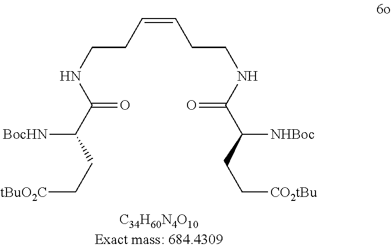 [SMILES: CC(C)(C)OC(=O)CC[C@H](NC(=O)OC(C)(C)C)C(=O)NCC/C=C\CCNC(=O)[C@H](CCC(=O)OC(C)(C)C)NC(=O)OC(C)(C)C]

Following the procedure for (4), the homodimerization product (6o) was obtained when homoallyl-modified glutamate (5o) (0.17 g, 0.48 mmol) was reacted with catalyst Ru-1 or Ru-2 (357 μl of a 0.10 M solution in THF) in THF (0.83 mL) under Ar(g). The residue was purified by column chromatography (SiO₂; 0% to 50% EtOAc in hexane) to afford 0.24 g (74%) of the product (6o) as a white solid. ¹H NMR (500 MHz, CDCl₃) δ 7.19 (bs, 2H), 5.46 (d, J=8.3 Hz, 2H), 5.42-5.36 (m, 2H), 4.18-4.14 (m, 2H), 3.70-3.64 (m, 2H), 2.91-2.90 (m, 2H), 2.32-2.29 (m, 4H), 2.25-2.13 (m, 4H), 2.01-1.96 (m, 2H), 1.93-1.83 (m, 2H), 1.41 (s, 36H). ¹³C NMR (126 MHz, CDCl₃) δ 172.31 (2C), 172.01 (2C), 155.91 (2C), 129.04 (2C), 80.47 (2C), 79.85 (2C), 54.15 (2C), 38.70 (2C), 31.94 (2C), 28.31 (6C), 28.04 (6C), 27.98 (2C), 27.84 (2C). HRMS (ESI) m/z calcd for C₃₂H₅₆N₄O₁₀ [M+H]⁺: 685.4309. found 685.4312.

### di-tert-Butyl((5S,16S)-3,6,15,18-tetraoxo-1,1,1,20,20,20-hexaphenyl-2,7,14,19-tetraazaicos-10-ene-5,16-diyl)dicarbamate (6p)

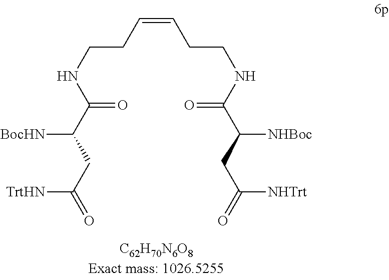

Following the procedure for (4), the homodimerization product (6p) was obtained when homoallyl-modified asparagine (5p) (0.18 g, 0.34 mmol) was reacted with catalyst Ru-1 or Ru-2 (255 μl of a 0.10 M solution in THF) in THF (0.60 mL) under Ar(g). The residue was purified by column chromatography (SiO₂; 0% to 66% EtOAc in hexane) to afford 0.24 g (70%) of the product (6p) as a white solid. ¹H NMR (500 MHz, CDCl₃) δ 7.30-7.22 (m, 18H), 7.20-7.15 (m, 12H), 7.09 (bs, 2H), 6.84-6.71 (m, 2H), 6.19 (d, J=7.7 Hz, 2H), 5.45-5.31 (m, 2H), 4.40-4.37 (m, 2H), 3.26-3.18 (m, 4H), 2.97-2.94 (m, 2H), 2.57-2.43 (m, 2H), 2.24-2.09 (m, 4H), 1.41 (s, 18H). ¹³C NMR (126 MHz, CDCl₃) δ 171.20 (2C), 170.37 (2C), 144.31 (6C), 128.64 (12C), 128.61 (2C), 128.43 (2C), 127.99 (2C), 127.93 (12C), 127.90 (2C), 127.04 (6C), 70.68 (2C), 39.21 (2C), 29.70 (2C), 28.32 (6C), 27.29 (2C). HRMS (ESI) m/z calcd for C₆₂H₇₀N₆O₈ [M+H]⁺: 1027.5255. found 1027.5251.

### di-tert-Butyl((6S,17S)-3,7,16,20-tetraoxo-1,1,1,22,22,22-hexaphenyl-2,8,15,21-tetraazadocos-11-ene-6,17-diyl)dicarbamate (6q)

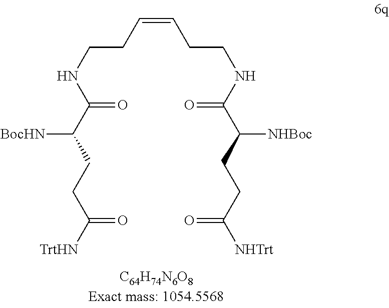

Following the procedure for (4), the homodimerization product (6q) was obtained when homoallyl-modified glutamine (5q) (0.14 g, 0.26 mmol) was reacted with catalyst Ru-1 or Ru-2 (194 μl of a 0.10 M solution in THF) in THF (0.45 mL) under Ar(g). The residue was purified by column chromatography (SiO₂; 3:1 EtOAc:hexanes) to afford 0.20 g (74%) of the product (6q) as a white solid. ¹H NMR (500 MHz, CDCl₃) δ 7.34-7.26 (m, 12H), 7.25-7.20 (m, 18H), 7.15 (bs, 2H), 6.75-6.64 (m, 2H), 5.62 (d, J=7.7 Hz, 2H), 5.36-5.30 (m, 2H), 4.01-3.97 (m, 2H), 3.25-3.20 (m, 2H), 3.15-3.12 (m, 2H), 2.40-2.27 (m, 4H), 2.18-2.10 (m, 4H), 2.01-1.91 (m, 2H), 1.87-1.82 (m, 3H), 1.42 (s, 18H); ¹³C NMR (126 MHz, CDCl₃) δ 171.77 (2C), 155.91 (2C), 144.57 (6C), 128.83 (2C), 128.69 (12C), 127.95 (2C), 127.90 (12C), 126.94 (6C), 79.76 (2C), 70.53 (2C), 53.60 (2C), 38.78 (2C), 33.65 (2C), 29.75 (2C), 28.35 (6C), 27.60 (2C). HRMS (ESI) m/z calcd for C₆₄H₇₄N₆O₈ [M+H]⁺: 1055.5568. found 1055.5549.

### tetra-tert-Buty′((5S,5′S)-(hex-3-ene-1,6-diylbis(azanediyl))bis(6-oxohexane-6, 1,5-triyl))tetracarbamate (6r)

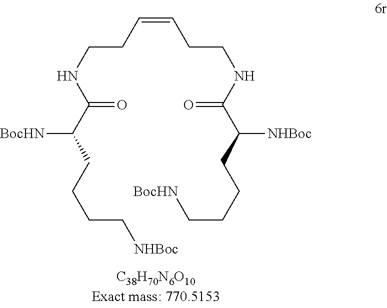 [SMILES: CC(C)(C)OC(=O)NCCCC[C@H](NC(=O)OC(C)(C)C)C(=O)NCC/C=C\CCNC(=O)[C@H](CCCCNC(=O)OC(C)(C)C)NC(=O)OC(C)(C)C]

Following the procedure for (4), the homodimerization product (6r) was obtained when homoallyl-modified lysine (5r) (0.21 g, 0.53 mmol) was reacted with catalyst Ru-1 or Ru-2 (394 μl of a 0.10 M solution in THF) in THF (0.92 mL) under Ar(g). The residue was purified by column chromatography (SiO₂; 3:1 EtOAc:hexanes) to afford 0.32 mg (78%) of the product 6r as a white solid. ¹H NMR (500 MHz, CDCl₃) δ 7.24 (bs, 1H), 5.43-5.39 (m, 2H), 5.26 (d, J=8.7 Hz, 2H), 4.68 (bs, 2H), 4.17-4.13 (m, 2H), 3.78-3.74 (m, 2H), 3.11-3.07 (m, 4H), 2.89-2.85 (m, 2H), 2.32-2.11 (m, 4H), 1.70 (m, 4H), 1.65-1.54 (m, 4H), 1.48-1.46 (m, 4H) 1.44 (s, 18H), 1.43 (s, 18H), 1.33-1.21 (m, 4H); ¹³C NMR (126 MHz, CDCl₃) δ 172.67 (2C), 156.04 (4C), 129.32 (2C), 79.89 (4C), 54.45 (2C), 40.15 (2C), 38.47 (2C), 32.34 (2C), 29.69 (2C), 29.63 (2C), 28.44 (6C), 28.34 (6C), 22.86 (2C). HRMS (ESI) m/z calcd for C₃₈H₇₀N₆O₁₀ [M+H]⁺: 771.5153. found 771.5138.

### Di-tert-butyl((6S,17S)-1,22-diimino-7,16-dioxo-1,22-bis((2,2,4,6,7-pentamethyl-2,3-dihydrobenzofuran)-5-sulfonamido)-2,8,15,21-tetraazadocos-11-ene-6,17-diyl)dicarbamate (6s)

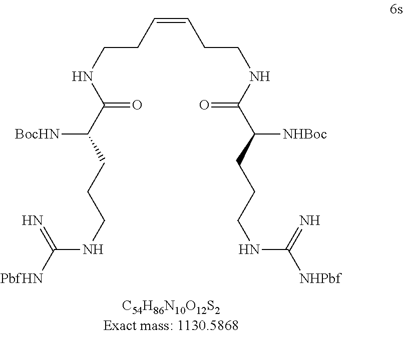

Following the procedure for (4), the homodimerization product (6s) was obtained when homoallyl-modified arginine (5s) (0.14 g, 0.24 mmol) was reacted with catalyst Ru-1 or Ru-2 (181 μl of a 0.10 M solution in THF) in THF (0.42 mL) under Ar(g). The residue was purified by column chromatography (SiO₂; 0% to 2% MeOH in EtOAc) to afford 93 mg (34%) of the product (6s) as a white solid. ¹H NMR (500 MHz, CD₃OD) δ 7.95-7.93 (m, 1H), 7.87-7.85 (m, 1H), 5.44-5.42 (m, 2H), 4.09-3.95 (m, 2H), 3.28-3.07 (m, 8H), 2.99 (s, 4H), 2.57 (s, 6H), 2.51 (s, 6H), 2.30-2.25 (m, 2H), 2.18-2.16 (m, 2H), 2.07 (s, 6H), 1.73-1.69 (m, 2H), 1.63-1.49 (m, 6H), 1.44 (s, 12H) 1.42 (s, 18H); ¹³C NMR (126 MHz, CD₃OD) δ 176.57 (2C), 173.66 (2C), 158.45 (2C), 156.69 (2C), 156.33 (2C), 137.98 (2C), 132.91 (2C), 132.09 (2C), 124.59 (2C), 117.02 (2C), 86.25 (2C), 79.19 (2C), 54.37 (2C), 42.57 (2C), 39.93 (2C), 38.69 (2C), 32.21 (2C), 29.42 (2C), 27.36 (4C), 27.33 (6C), 25.74 (2C), 18.22 (2C), 17.04 (2C), 11.14 (2C). HRMS (ESI) m/z calcd for C₅₄H₈₆N₁₀O₁₂S [M+H]⁺: 1131.5868. found 1131.5877.

### General Procedure for Cross Metathesis of Amino Acids

### tert-Butyl((6S,17S)-6-isopropyl-2,2-dimethyl-4,7,16-trioxo-3-oxa-5,8,15-triazaoctadec-11-en-17-yl)carbamate (7)

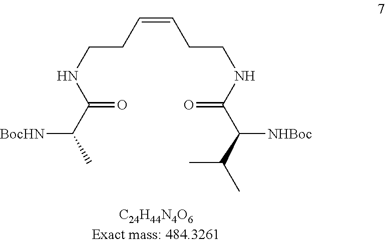 [SMILES: CC(C)[C@H](NC(=O)OC(C)(C)C)C(=O)NCC/C=C\CCNC(=O)[C@H](C)NC(=O)OC(C)(C)C]

A round bottom flask was charged with Boc-protected homoallyl alanine (3) (50 mg, 0.20 mmol) and the cross partner homoallyl valine (5a) (223 mg, 0.80 mmol, 4 eq.) under a gentle stream of Ar(g). To this was added anhydrous THF (0.40 mL). A solution of catalyst Ru-1 or Ru-2 (155 μl of a 0.10 M solution in THF) was added and the reaction mixture was heated to 40° C. and stirred for 4h. The solution was cooled to room temperature and then quenched with an excess of ethyl vinyl ether (0.50 mL, 5.2 mmol). The solvent was removed in vacuo and the residue purified by column chromatography (SiO₂; 0% to 66% EtOAc in hexane) to afford 61 mg (60%) of product 7 as a white solid. ¹H NMR (500 MHz, CDCl₃) δ 7.24 (bs, 1H), 5.46-5.36 (m, 2H), 5.29-5.24 (m, 2H), 4.34-4.25 (m, 1H), 3.90 (dd, J=9.4, 7.4 Hz, 1H), 3.78-3.73 (m, 2H), 2.94-2.82 (m, 2H), 2.31-2.12 (m, 4H), 1.98-1.89 (m, 2H), 1.43 (s, 18H), 1.33 (d, J=7.0 Hz, 3H), 0.94 (dd, J=6.8, 4.5 Hz, 6H); ¹³C NMR (126 MHz, CDCl₃) δ 173.49, 172.14, 156.21, 155.76, 129.25 (2C), 79.85, 79.72, 60.33, 50.09, 38.63, 38.41, 30.94, 29.67, 28.33 (6C), 28.03, 19.24, 18.51 (2C). HRMS (ESI) m/z calcd for C₂₄H₄₄N₄O₆ [M+H]⁺: 485.3261. found 485.3258.

### tert-Butyl((6S,17S,Z)-2,2-dimethyl-4,7,16-trioxo-6-((3-((2,2,4,6,7-pentamethyl-2,3-dihydrobenzofuran-5-yl)sulfonyl)guanidino)methyl)-3-oxa-5,8,15-triazaoctadec-11-en-17-yl)carbamate (8)

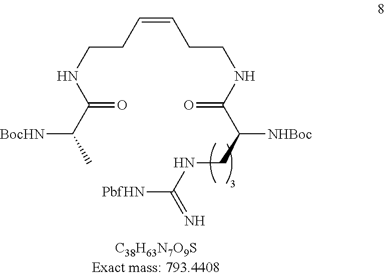

Following the procedure for (7), the cross product (8) was obtained when homoallyl-modified arginine (5s) (50 mg, 0.086 mmol) was reacted with alanine (3) (84 mg, 0.35 mmol, 4 eq.) in THF (0.17 mL) in the presence of catalyst Ru-1 or Ru-2 (65 μl of a 0.10 M solution in THF) under Ar(g). The residue was purified by column chromatography (SiO₂; 0% to 2% MeOH in EtOAc) to afford 28 mg (41%) of the product (6s) as a white solid. ¹H NMR (500 MHz, CDCl₃) δ 7.13 (bs, 1H), 6.93 (bs, 1H), 6.35 (bs, 2H), 5.74 (bs, 1H), 5.65 (bs, 1H), 5.45-5.36 (m, 2H), 4.34-4.13 (m, 2H), 3.40-3.16 (m, 6H), 2.95 (s, 2H), 2.59 (s, 3H), 2.52 (s, 3H), 2.24-2.17 (m, 4H), 2.09 (s, 3H), 1.77 (bs, 1H), 1.61-1.53 (m, 3H), 1.46 (s, 6H), 1.41 (s, 18H), 1.33 (d, J=7.0 Hz, 3H). ¹³C NMR (126 MHz, CDCl₃) δ 173.70, 172.70, 158.68, 156.45, 155.77 (2C), 138.29, 133.17, 132.25, 129.77 (2C), 129.26, 124.51, 117.40, 86.27, 79.98 (2C), 53.83, 50.38, 43.29, 40.28, 38.91, 38.60, 32.61, 30.29, 28.54 (2C), 28.36 (3C) 28.35 (3C), 25.51, 19.20, 18.47, 17.86, 12.37. HRMS (ESI) m/z calcd for C₂₄H₄₄N₄O₆ [M+H]⁺: 794.4408. found 794.4411.

### Synthesis of Allyl-Modified Amino Acids

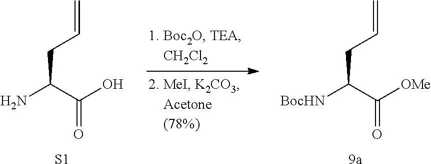

### Methyl (S)-2-((tert-butoxycarbonyl)amino)pent-4-enoate (9a)

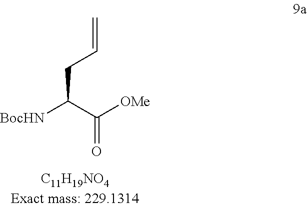 [SMILES: C=CC[C@H](NC(=O)OC(C)(C)C)C(=O)OC]

The Boc-protected allyl glycine (9a) was synthesized using a two-step procedure starting from allyl glycine. Briefly, to a stirring suspension of(S)-allyl glycine S1 (2.0 g, 17.3 mmol) in CH₂C₁₂ (25 mL) was added triethylamine (TEA, 1.9 mL, 26.0 mmol, 1.5 eq.) under Ar(g). The solution was cooled to 0° C. by immersion in an ice bath. Di-tert-butyl dicarbonate (5.6 g, 26.0 mmol, 1.5 eq.) was dissolved in CH₂C₁₂ (10 mL) and added dropwise to the stirring solution. The reaction was removed from the ice bath and allowed to stir at room temperature for 12 h. The crude mixtures was diluted with H₂O (10 mL) and extracted with 1 M HCl (3×10 mL), brine (3×10 mL), and dried over Na₂SO₄. The solvent was removed in vacuo to afford a light yellow oil which was carried on to the next step without further purification.

To the oil was added acetone (20 mL) and solid K₂CO₃ (4.8 g, 34.6 mmol, 2 eq.) at room temperature. The reaction was stirred for 10 min, followed by the addition of iodomethane (2.2 mL, 34.6 mmol, 2 eq.) and the mixture stirred for 12 h. The solvent was evaporated and the residue taken up in EtOAc (25 mL) and washed with saturated Na₂S₂O₃ (2×20 mL), brine (2×20 mL), and dried over Na₂SO₄. The solvent was removed in vacuo and the crude residue was purified by flash chromatography (3:1 Hex:EtOAc) to afford 3.1 g (78%) of (9a) as a colorless oil. ¹H NMR (500 MHz, CDCl₃) δ 5.64 (ddt, J=16.5, 10.7, 7.2 Hz, 1H), 5.15-4.99 (m, 3H), 4.39-4.25 (m, 1H), 3.68 (s, 3H), 2.56-2.35 (m, 2H), 1.39 (s, 9H); ¹³C NMR (126 MHz, CDCl₃) δ 172.47, 155.13, 132.29, 118.95, 79.76, 52.86, 52.14, 36.69, 28.22 (3C). HRMS (ESI) m/z calcd for C₁₁H₁₉NO₄ [M+H]⁺: 230.1314. found 230.1317.

### Synthesis of Homoallyl-Modified Amino Acids

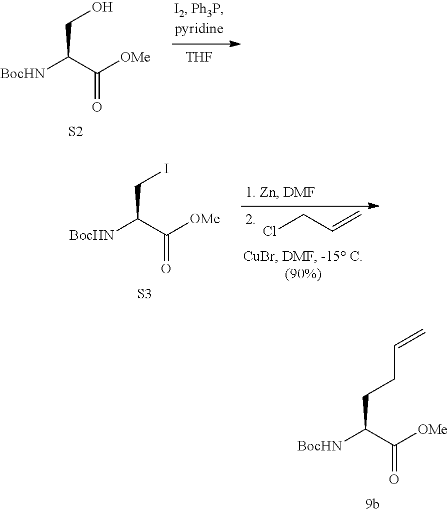

### Methyl (S)-2-((tert-butoxycarbonyl)amino)hex-5-enoate (9b)

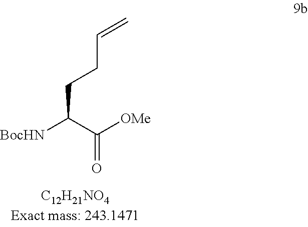 [SMILES: C=CCC[C@H](NC(=O)OC(C)(C)C)C(=O)OC]

Boc-homoallyl glycine (9b) was synthesized using a three-step protocol from commercially available Boc-Ser-OMe (S2). In a typical procedure, a flask was charged with Boc-Ser-OMe (2.0 g, 9.1 mmol) and triphenylphosphine (3.6 g, 13.7 mmol, 1.5 eq.) under Ar(g). To this was added THF (20 mL) and the solution cooled to 0° C. by immersion in an ice bath. Pyridine (1.5 mL, 18.2 mmol, 2 eq.) was added dropwise, followed by solid iodine (3.5 g, 13.7 mmol, 1.5 eq.) in three portions at 0° C. The ice bath was removed and stirring was continued for 4 h at room temperature. The mixture was extracted with Et₂O (3×20 mL). The combined organic layers were washed with 1M HCl (3×20 mL), 1M Na₂S₂O₃ (2×20 mL), brine (2×20 mL) and dried over Na₂SO₄, filtered and concentrated in vacuo. The crude residue was of sufficient purity to be used in the next step without further purification.

The iodopropanoate S3 was dissolved in DMF (5 mL) and added dropwise to a flask containing activated zinc (2.4 g, 36.4 mmol, 4 eq.) at 0° C. under Ar(g). The reaction mixture was removed from the ice bath and allowed to stir at room temperature for 3 h, upon which TLC (4:1 petroleum ether: EtOAc) indicated loss of starting material and formation of a lower R_{f} spot. At this point, the reaction mixture was stopped to let the solid settle to the bottom. The supernatant was then carefully transferred by syringe to a suspension of copper (I) bromide (0.26 g, 1.8 mmol) in DMF (mL) at −15° C. that also contained allyl chloride (1.3 mL, 15.5 mmol, 1.7 eq.). After complete addition, the cooling bath was removed and stirring was continued overnight. At this point, EtOAc (20 mL) was added to the reaction mixture and stirring was continued for 15 min. To the mixture was added H₂O (20 mL), the organic layer was removed and successively washed with 1M Na₂S₂O₃ (2×20 mL), H₂O (2×20 mL), brine (2×20 mL), and dried over Na₂SO₄, filtered and concentrated in vacuo. The crude residue was purified by flash chromatography (SiO₂, 8:1 petroleum ether:EtOAc) to afford 2.0 g (90%) of (9b) as a colorless oil. ¹H NMR (500 MHz, CDCl₃) δ 5.72 (ddt, J=16.9, 10.2, 6.6 Hz, 1H), 5.18-5.07 (m, 1H), 5.01-4.90 (m, 2H), 4.26-4.23 (m, 1H), 3.67 (s, 3H), 2.08-2.01 (m, 2H), 1.88-1.79 (m, 1H), 1.70-1.61 (m, 1H), 1.37 (s, 9H); ¹³C NMR (126 MHz, CDCl₃) δ 173.11, 155.23, 136.87, 115.50, 79.64, 52.09, 51.99, 31.85, 29.39, 28.21 (3C). HRMS (ESI) m/z calcd for C₁₂H₂₁NO₄ [M+H]⁺: 244.1471. found 244.1474.

### Methyl O-allyl-N-(tert-butoxycarbonyl)-L-serine (9c)

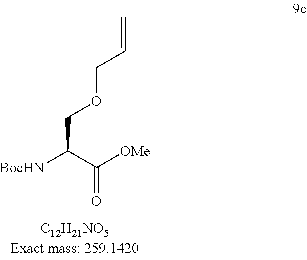 [SMILES: C=CCOC[C@H](NC(=O)OC(C)(C)C)C(=O)OC]

A solution of Boc-Ser-OMe S2 (2.0 g, 9.1 mmol) in anhydrous THF (40 mL) was degassed and treated with allylmethyl carbonate (1.4 mL, 12.7 mmol, 1.4 eq). Tetrakis(triphenylphosphine)palladium (0.21 g, 0.18 mmol, 0.02 eq.) was added and the reaction mixture heated to 60° C. for 4 h upon which TLC (2:1 EtOAc:hexanes) indicated loss of starting material. The solvent was removed under reduced pressure and the residue was diluted with EtOAc (30 mL) and washed with NaHCO₃ (2×30 mL) and brine (30 mL). The organic layer was dried over MgSO₄, filtered, and concentrated under reduced pressure. The residue was purified by column chromatography (SiO₂; 0% to 66% EtOAc in hexane) to afford 1.6 g (68%) of the product (9c) as a clear oil. ¹H NMR (500 MHz, CDCl₃) δ 5.79 (ddt, J=17.3, 10.4, 5.6 Hz, 1H), 5.41-5.31 (m, 1H), 5.25-5.10 (m, 2H), 4.40-4.37 (m, 1H), 3.95-3.92 (m, 2H), 3.80 (dd, J=9.5, 3.3 Hz, 1H), 3.71 (s, 3H), 3.61 (dd, J=9.5, 3.4 Hz, 1H), 1.41 (s, 9H); ¹³C NMR (126 MHz, CDCl₃) δ 171.11, 155.42, 134.01, 117.29, 79.85, 72.14, 69.86, 53.92, 52.37, 28.25 (3C). HRMS (ESI) m/z calcd for C₁₂H₂₁N₅O₅ [M+H]⁺: 260.1420. found 260.1428.

### Methyl S-allyl-N-(tert-butoxycarbonyl)-L-cysteine (9d)

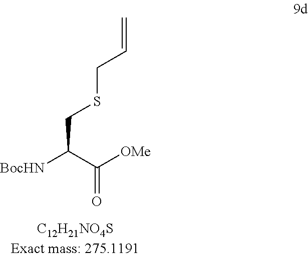 [SMILES: C=CCSC[C@H](NC(=O)OC(C)(C)C)C(=O)OC]

Following the procedure for (9c), the allyl-protected cysteine (9d) was obtained when Boc-Cys-OMe (1.8 g, 7.6 mmol) was treated with allylmethyl carbonate (1.2 mL, 10.7 mmol, 1.4 eq.) and tetrakis(triphenylphosphine)palladium (0.17 g, 0.15 mmol, 0.02 eq.) in THF (30 mL). The residue was purified by column chromatography (SiO₂; 0% to 25% EtOAc in hexane) to afford 1.4 g (69%) of the product (9d) as a clear oil. ¹H NMR (500 MHz, CDCl₃) δ 5.70 (ddt, J=16.9, 9.6, 7.2 Hz, 1H), 5.38-5.29 (m, 1H), 5.12-5.04 (m, 2H), 4.48-4.46 (m, 1H), 3.72 (s, 3H), 3.13-3.03 (m, 2H), 2.88 (dd, J=13.9, 5.0 Hz, 1H), 2.80 (dd, J=13.9, 5.7 Hz, 1H), 1.41 (s, 9H); ¹³C NMR (126 MHz, CDCl₃) δ 171.55, 155.06, 133.62, 117.78, 80.00, 53.10, 52.45, 35.07, 32.76, 28.25 (3C). HRMS (ESI) m/z calcd for C₁₂H₂₁NO₄S [M+H]⁺: 276.1191. found 276.1188.

### Procedure for Homodimerization of Allyl-Modified Amino Acids

### Dimethyl (2S,7S,Z)-2,7-bis((tert-butoxycarbonyl)amino)oct-4-enedioate (10a)

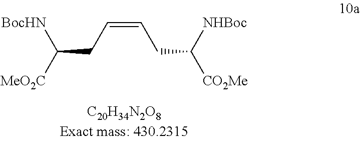 [SMILES: COC(=O)[C@H](C/C=C\C[C@H](NC(=O)OC(C)(C)C)C(=O)OC)NC(=O)OC(C)(C)C]

Allyl-modified glycine (9a) (0.18 g, 0.79 mmol) was dissolved in THF (1.5 mL) under a gentle stream of Ar (g). A solution of catalyst Ru-1 or Ru-2 in THF (588 μl of a 0.10 M solution in THF) was added and the reaction heated to 40° C. and stirred for 4 h. The solution was allowed to cool to room temperature upon which an excess of ethyl vinyl ether (0.5 mL, 5.2 mmol) was added to quench the reaction. The solvent was removed in vacuo and the residue purified by column chromatography (SiO₂; 0% to 25% EtOAc in hexane) to afford 0.15 g (45%) of product 10a as a clear oil. ¹H NMR (500 MHz, CDCl₃) δ 5.50-5.46 (m, 2H), 5.17 (d, J=8.3 Hz, 2H), 4.44-4.40 (m, 2H), 3.75 (s, 6H), 2.62-2.54 (m, 2H), 2.47-2.42 (m, 2H), 1.45 (s, 18H); ¹³C NMR (126 MHz, CDCl₃) δ 172.36 (2C), 155.09 (2C), 127.32 (2C), 109.99 (2C), 80.11 (2C), 52.87 (2C), 52.36 (2C), 30.40 (3C), 28.27 (3C). HRMS (ESI) m/z calcd for C₂₀H₃₄N₂O₈ [M+H]⁺: 431.2315. found 431.2318.

### Dimethyl (2S,9S,Z)-2,9-bis((tert-butoxycarbonyl)amino)dec-5-enedioate (10b)

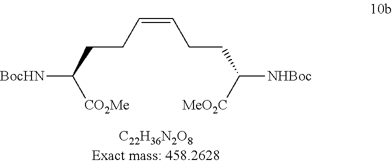 [SMILES: COC(=O)[C@H](CC/C=C\CC[C@H](NC(=O)OC(C)(C)C)C(=O)OC)NC(=O)OC(C)(C)C]

Following the procedure for (10a), the homodimerization product (10b) was obtained when homoallyl-modified glycine (9b) (0.13 g, 0.53 mmol) was reacted with catalyst Ru-1 or Ru-2 (395 μl of a 0.10 M solution in THF) in THF (1.1 mL) under Ar(g). The residue was purified by column chromatography (SiO₂; 3:1 EtOAc:hexanes) to afford 0.14 g (58%) of the product (10b) as a clear oil. ¹H NMR (300 MHz, CDCl₃) δ 5.43-5.37 (m, 2H), 5.07 (d, J=8.4 Hz, 2H), 4.32-4.25 (m, 2H), 3.74 (s, 6H), 2.12-2.04 (m, 4H), 1.94-1.76 (m, 2H), 1.74-1.64 (m, 2H), 1.48 (s, 18H); ¹³C NMR (126 MHz, CDCl₃) δ 173.15 (2C), 155.34 (2C), 129.31 (2C), 79.89 (2C), 63.96 (2C), 52.13 (2C), 32.48 (2C), 28.30 (6C), 23.18 (2C). HRMS (ESI) m/z calcd for C₂₂H₃₈N₂O₈ [M+H]⁺: 459.2628. found 459.2631.

### Methyl (6S,15S,Z)-15-((tert-butoxycarbonyl)amino)-6-(methoxycarbonyl)-2,2-dimethyl-4-oxo-3,8,13-trioxa-5-azahexadec-10-en-16-oate (10c)

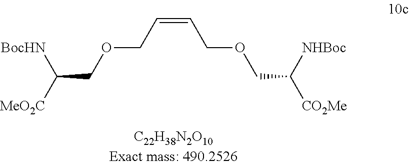 [SMILES: COC(=O)[C@H](COC/C=C\COC[C@H](NC(=O)OC(C)(C)C)C(=O)OC)NC(=O)OC(C)(C)C]

Following the procedure for (10a), the homodimerization product (10c) was obtained when allyl-modified serine (9c) (0.14 g, 0.54 mmol) was reacted with catalyst Ru-1 or Ru-2 (400 μl of a 0.10 M solution in THF) in THF (1.0 mL) under Ar(g). The residue was purified by column chromatography (SiO₂; 0% to 33% EtOAc in hexanes) to afford 0.17 g (67%) of the product (10c) as a clear oil. ¹H NMR (500 MHz, CDCl₃) δ 5.67-5.62 (m, 2H), 5.37 (d, J=8.8 Hz, 2H), 4.46-4.39 (m, 2H), 4.05-3.99 (m, 4H), 3.85-3.81 (m, 2H), 3.76 (s, 6H), 3.66-3.60 (m, 2H), 1.45 (s, 18H); ¹³C NMR (126 MHz, CDCl₃) δ 171.08 (2C), 155.44 (2C), 129.06 (2C), 80.02 (2C), 70.15 (2C), 66.97 (2C), 53.92 (2C), 52.49 (2C), 28.31 (6C). HRMS (ESI) m/z calcd for C₂₂H₃₈N₂O₁₀ [M+H]⁺: 491.2526. found 491.2533.

### Methyl (6R,15R,Z)-15-((tert-butoxycarbonyl)amino)-6-(methoxycarbonyl)-2,2-dimethyl-4-oxo-3-oxa-8,13-dithia-5-azahexadec-10-en-16-oate (10d)

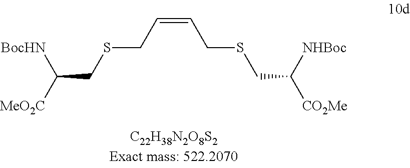 [SMILES: COC(=O)[C@H](CSC/C=C\CSC[C@H](NC(=O)OC(C)(C)C)C(=O)OC)NC(=O)OC(C)(C)C]

Following the procedure for (10a), the homodimerization product (10d) was obtained when allyl-modified cysteine (9d) (0.15 g, 0.54 mmol) was reacted with catalyst Ru-1 or Ru-2 (400 μl of a 0.1 M solution in THF) in THF (1.0 mL) under Ar(g). The residue was purified by column chromatography (SiO₂; 0% to 33% EtOAc in hexanes) to afford 0.20 g (71%) of the product (10d) as a clear oil. ¹H NMR (300 MHz, CDCl₃) δ 5.60 (td, J=5.1, 2.5 Hz, 2H), 5.40 (d, J=8.1 Hz, 2H), 4.60-4.45 (m, 2H), 3.76 (s, 6H), 3.29-3.15 (m, 4H), 2.90 (m, 4H), 1.45 (s, 18H); ¹³C NMR (126 MHz, CDCl₃) δ 171.54 (2C), 155.16 (2C), 128.31 (2C), 80.17 (2C), 53.31 (2C), 52.57 (2C), 33.95 (2C), 28.76 (2C), 28.31 (6C). HRMS (ESI) m/z calcd for C₂₂H₃₈N₂O₈S2 [M+H]⁺: 523.3070. found 523.3081.

### Procedure for Cross Metathesis of Allyl Modified Amino Acids and Allyl Acetate

### Methyl (S,Z)-6-acetoxy-2-((tert-butoxycarbonyl)amino)hex-4-enoate (12a)

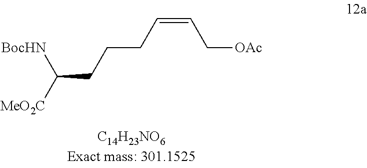 [SMILES: COC(=O)[C@H](CCC/C=C\COC(C)=O)NC(=O)OC(C)(C)C]

Boc-protected allyl glycine (9a) (0.15 g, 0.65 mmol) was dissolved in THF (1.1 mL) under a gentle stream of Ar(g). To this was added allyl acetate (0.35 mL, 3.3 mmol, 5 eq.), followed by a solution of catalyst Ru-1 or Ru-2 (490 μl of a 0.10 M solution in THF). The reaction mixture was heated to 40° C. and stirred for 4h. The solution was cooled to room temperature and then quenched with an excess of ethyl vinyl ether (0.5 mL, 5.2 mmol). The solvent was removed in vacuo and the residue purified by column chromatography (SiO₂; 0% to 25% EtOAc in hexane) to afford 83 mg (42%) of product (12a) as a clear, colorless oil; ¹H NMR (500 MHz, CDCl₃) δ 5.71-5.68 (m, 1H), 5.59-5.53 (m, 1H), 5.20 (d, J=8.4 Hz, 1H), 4.63-4.52 (m, 2H), 4.42-4.32 (m, 1H), 3.73 (s, 3H), 2.69-2.49 (m, 2H), 2.05 (s, 3H), 1.42 (s, 9H); ¹³C NMR (126 MHz, CDCl₃) δ 172.21, 170.69, 155.17, 128.61, 127.40, 79.90, 59.77, 52.88, 52.26, 30.42, 28.25 (3C), 20.82. HRMS (ESI) m/z calcd for C₁₄H₂₃NO₆ [M+H]⁺: 302.1525. found 302.1588.

### Methyl (S,Z)-7-acetoxy-2-((tert-butoxycarbonyl)amino)hept-5-enoate (12b)

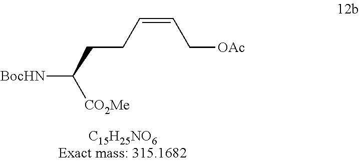 [SMILES: COC(=O)[C@H](CC/C=C\COC(C)=O)NC(=O)OC(C)(C)C]

Following the procedure for (12a), the cross product (12b) was obtained when homoallyl-modified glycine (9b) (0.14 g, 0.57 mmol) in THF (1.0 mL) was reacted with catalyst Ru-1 or Ru-2 (431 μl of a 0.10 M solution in THF) in the presence of excess allyl acetate (0.31 mL, 2.9 mmol, 5 eq.). The residue was purified by column chromatography (SiO₂; 0% to 20% EtOAc in hexane) to afford 0.10 g (56%) the product (12b) as a clear oil. ¹H NMR (300 MHz, CDCl₃) δ 5.65-5.48 (m, 2H), 5.08 (d, J=8.5 Hz, 1H), 4.66-4.51 (m, 2H), 4.32-4.26 (m, 1H), 3.72 (s, 3H), 2.23-2.09 (m, 2H), 2.04 (s, 3H), 1.97-1.80 (m, 1H), 1.78-1.61 (m, 1H), 1.43 (s, 9H); ¹³C NMR (126 MHz, CDCl₃) δ 172.96, 170.70, 155.28, 133.10, 124.85, 79.90, 60.04, 52.96, 52.16, 32.34, 28.26 (3C), 23.47, 20.82. HRMS (ESI) m/z calcd for C₁₅H₂₅NO₆ [M+H]⁺: 316.1682. found 316.1690.

### Methyl (Z)—O-(4-acetoxybut-2-en-1-yl)-N-(tert-butoxycarbonyl)-L-serine (12c)

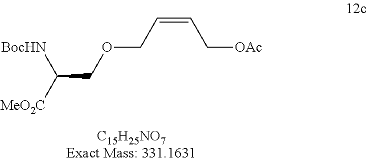 [SMILES: COC(=O)[C@H](COC/C=C\COC(C)=O)NC(=O)OC(C)(C)C]

Following the procedure for (12a), the cross product (12c) was obtained when homoallyl-modified serine (9c) (0.13 g, 0.50 mmol) in THF (0.88 mL) was reacted with catalyst Ru-1 or Ru-2 (375 μl of a 0.10 M solution in THF) in the presence of excess allyl acetate (0.27 mL, 2.5 mmol, 5 eq.). The residue was purified by column chromatography (SiO₂; 0% to 33% EtOAc in hexane) to afford 0.11 g (63%) of the product (12c) as a clear oil. ¹H NMR (500 MHz, CDCl₃) δ 5.74-5.65 (m, 2H), 5.43-5.35 (m, 1H), 4.63-4.58 (m, 2H), 4.46-4.41 (m, 1H), 4.13-4.04 (m, 2H), 3.90-3.85 (m, 1H), 3.77 (s, 3H), 3.68-3.62 (m, 1H), 2.07 (s, 3H), 1.46 (s, 9H); ¹³C NMR (126 MHz, CDCl₃) δ 173.14, 172.71, 155.24, 136.90, 115.56, 79.77, 53.00, 52.13, 31.97, 29.41, 28.26 (3C), 28.23, 18.56. HRMS (ESI) m/z calcd for C₁₅H₂₅NO₇ [M+H]⁺: 332.1631. found 332.1638.

### Methyl (Z)—S-(4-acetoxybut-2-en-1-yl)-N-(tert-butoxycarbonyl)-L-cysteine (12d)

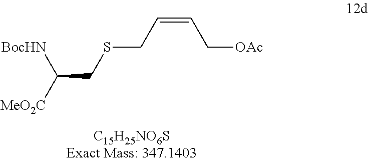 [SMILES: COC(=O)[C@H](CSC/C=C\COC(C)=O)NC(=O)OC(C)(C)C]

Following the procedure for (12a), the cross product (12d) was obtained when homoallyl-modified cysteine (9d) (0.14 g, 0.51 mmol) in THF (0.87 mL) was reacted with catalyst Ru-1 or Ru-2 (377 μl of a 0.10 M solution in THF) in the presence of excess allyl acetate (0.27 mL, 2.5 mmol, 5 eq.). The residue was purified by column chromatography (SiO₂; 0% to 33% EtOAc in hexane) to afford 0.11 g (62%) of the product (12d) as a clear oil. ¹H NMR (500 MHz, CDCl₃) δ 5.73-5.63 (m, 2H), 5.38-5.30 (m, 1H), 4.68-4.58 (m, 2H), 4.57-4.51 (m, 1H), 3.77 (s, 3H), 3.28-3.21 (m, 2H), 2.95 (dd, J=13.8, 4.9 Hz, 1H), 2.87 (dd, J=13.8, 5.9 Hz, 1H), 2.06 (s, 3H), 1.45 (s, 9H); ¹³C NMR (126 MHz, CDCl₃) δ 171.41, 170.60, 155.09, 130.09, 126.61, 80.14, 59.55, 53.36, 52.44, 33.97, 29.08, 28.27 (3C), 20.81. HRMS (ESI) m/z calcd for C₁₅H₂₅NO₆S [M+H]⁺: 348.1403. found 348.1419.

### General Procedure for the Synthesis of Homoallyl-Modified Peptides

### Boc-Ser(OtBu)-Asp(OtBu)-Phe-Ile-Gln(Trt)-Val homoallyl peptide 13

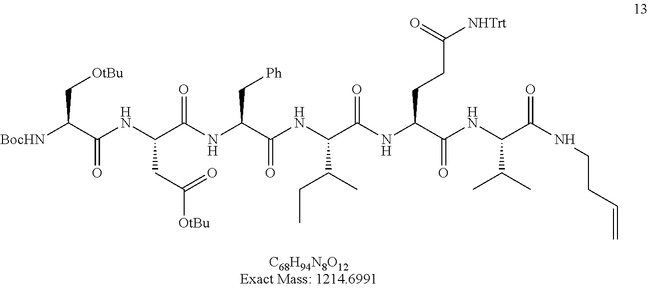

Peptide 13 was synthesized by solution phase methods using iterative coupling of Fmoc-protected amino acids. Briefly, Boc-protected homoallyl-modified valine (5a) (1.0 g, 3.7 mmol) was dissolved in a mixture of 1:1 TFA:DCM (4 mL) and allowed to stir for 4 h at room temperature upon which TLC (1:1 EtOAc:hexanes) indicated loss of starting material. The solution was diluted with CH₂C₁₂ (30 mL) and the solvent was removed in vacuo. The crude residue was dissolved in a mixture of DMF (10 mL) and N,N-diisopropylethylamine (DIEA, 5.3 mL, 30.0 mmol, 8 eq.) and allowed to stir at room temperature for 20 min. At this point, a solution of Fmoc-Gln(Trt)-OH (4.5 g, 7.4 mmol, 2 eq.), HOBt (1.0 g, 7.4 mmol, 2 eq.), HBTU (2.8 g, 7.4 mmol, 2 eq.), and DIEA (2.6 mL, 14.8 mmol, 4 eq.) in DMF (8 mL) was added to the stirring solution. The reaction mixture was heated to 50° C. and allowed to stir for 1 h. The solution was cooled to room temperature and quenched with H₂O (20 mL), and to this was added EtOAc (50 mL). The organic layer was removed and washed with H₂O (5×20 mL), brine (5×20 mL) and dried over MgSO₄. The solvent was removed in vacuo to afford the Fmoc-protected dipeptide as a white solid which was found to be of sufficient purity to be used in subsequent reactions.

The Fmoc-protected dipeptide (2.1 g, 2.7 mmol) was dissolved in a mixture of piperidine (3.0 mL, 30 mmol) in DMF (9.0 mL) and allowed to stir at room temperature for 1 h, upon which a white precipitate had formed. The precipitate was filter off, and the filtrate concentrated under reduced pressure. The crude filtrate was dissolved in EtOAc (50 mL) and extracted with H₂O (5×30 mL), brine (5×30 mL) and dried over MgSO₄. The solvent was removed in vacuo to afford a clear oil (1.3 g) which was used in the next step without further purification.

This iterative procedure was used for subsequent amino acid couplings, at each step monitoring the conversion by LC/MS. The termination of the peptide sequence was carried out using the requisite Boc-protected amino acid. After the final coupling, the crude peptide was dissolved in EtOAc (50 mL) and washed with H₂O (5×30 mL), brine (5×30 mL), and dried over MgSO₄. The solvent was removed in vacuo and the product purified by column chromatography (SiO₂; 1:1 DCM:EtOAc+1 to 5% MeOH) to afford a white solid (R_{f}=0.45 in 1:1 DCM:EtOAc+2% MeOH). ¹H NMR (500 MHz, CDCl₃+CD₃OD) δ 7.79 (d, J=8.3 Hz, 1H), 7.66 (d, J=8.3 Hz, 1H), 7.44-7.32 (m, 3H), 7.25-7.09 (m, 17H), 5.75-5.65 (m, 1H), 5.48 (d, J=Hz, 1H), 5.04-4.91 (m, 2H), 4.37-4.35 (m, 1H), 4.33-4.24 (m, 2H), 4.19-4.14 (m, 2H), 4.02-3.97 (m, 2H), 3.79-3.77 (m, 2H), 3.56-3.54 (m, 4H), 3.21-3.19 (m, 4H), 2.57-2.25 (m, 4H), 2.22-2.14 (m, 4H), 2.07-1.92 (m, 3H), 1.85-1.80 (m, 2H), 1.43-1.42 (m, 2H), 1.41 (s, 18H) 1.38-1.35 (m, 6H), 1.13-1.11 (m, 14H), 0.89-0.79 (m, 9H); ¹³C NMR (126 MHz, CDCl₃+CD₃OD) δ 172.87, 171.95, 171.93, 171.84, 171.63, 171.18, 170.52, 155.83, 144.50 (3C), 135.36, 129.03, 128.90, 128.70 (3C), 128.67 (3C), 128.57, 128.55, 127.71 (3C), 127.68, 126.77 (3C), 126.74, 126.60, 125.43, 117.87, 116.39, 110.47, 82.01, 80.70, 73.90, 73.53, 61.91, 59.06, 59.03, 58.96, 58.92, 54.02, 38.73, 35.76, 33.36, 33.33, 29.63, 28.22 (3C), 28.13, 27.88 (3C), 27.25, 27.23, 27.22, 27.17 (3C), 25.21, 19.21, 17.59, 17.55, 15.37, 11.03. HRMS (ESI) m/z calcd for C₆₈H₉₄N₈O₁₂ [M+H]⁺: 1215.7016. found 1215.7082.

### Boc-Lys(Boc)-Val-Leu-Tyr(OtBu)-Arg(Pbf)-Arg(Pbf) homoallyl peptide 14

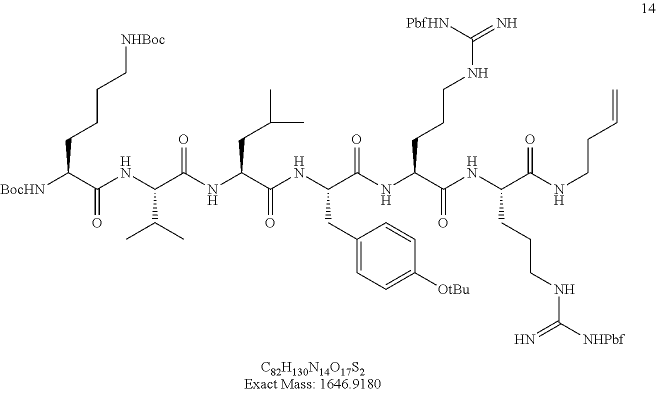

Peptide 14 was synthesized by iterative amino acid coupling in a manner analogous to that of peptide 13. Purification of the final peptide was achieved by column chromatography (SiO₂; 1:1 DCM:EtOAc+1 to 5% MeOH) to afford a clear gel. (R_{f}=0.55 in 1:1 DCM:EtOAc+10% MeOH). ¹H NMR (500 MHz, CDCl₃) δ 7.87 (d, J=6.7 Hz, 1H), 7.54-7.35 (m, 5H), 7.17 (bs, 1H), 7.10-7.07 (m, 2H), 6.79-6.77 (m, 2H), 6.61 (bs, 1H), 6.28 (bs, 4H), 5.72-5.63 (m, 1H), 5.42 (m, 1H), 4.98-4.89 (m, 2H), 4.29-4.15 (m, 3H), 3.19-3.06 (m, 8H), 2.99-2.95 (m, 2H), 2.88-2.84 (m, 8H), 2.78-2.77 (m, 3H), 2.47-2.45 (m, 6H), 2.40-2.39 (m, 6H), 2.20-2.10 (m, 3H), 1.98-1.96 (m, 6H), 1.81-1.50 (m, 12H), 1.38 (s, 9H), 1.36 (s, 9H), 1.34 (s, 9H), 1.20 (m, 12H), 0.93-0.86 (m, 6H), 0.77-0.74 (m, 4H), 0.68-0.66 (m, 4H); ¹³C NMR (126 MHz, CDCl₃+CD₃OD) δ 175.42, 174.51, 173.21, 172.91 (2C), 172.32, 162.89 (2C), 158.53, 157.30 (2C), 157.09, 156.43 (2C), 156.36, 153.99, 138.11 (2C), 135.18 (2C), 133.02, 132.06 (2C), 129.26 (2C), 124.45 (2C), 123.94 (2C), 117.32, 116.33, 86.29 (2C), 80.65, 79.30, 78.36, 61.40, 57.09, 56.46, 54.52, 54.31, 53.28, 43.12 (2C), 40.38 (2C), 39.49 (2C), 38.95 (2C), 36.44, 35.82, 33.24 (2C), 31.34, 28.65 (3C), 28.35 (3C), 28.29 (3C), 28.12 (2C), 25.31, 24.52, 22.55 (2C), 20.87 (2C), 18.98 (2C), 18.26 (2C), 17.69 (2C), 12.18 (2C). HRMS (ESI) m/z calcd for C₈₂H₁₃₀N₁₄O₁₇S₂ [M+H]⁺: 1648.9180. found 1648.9332.

### Procedure for the Synthesis of Peptide 15 by Cross Metathesis

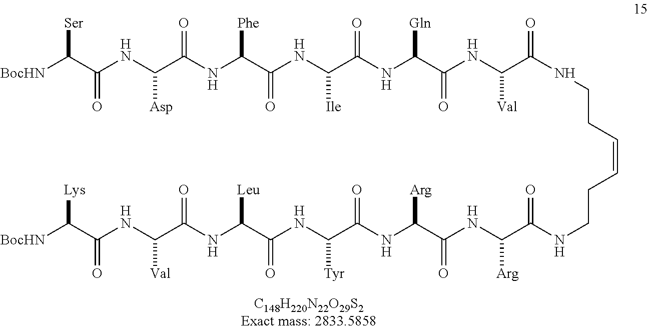

A 1 mL vial was charged with peptides 13 (23 mg, 0.020 mmol) and 14 (33 mg, 0.020 mmol) under a gentle stream of Ar(g). To this was added THF (150 μL), followed by the addition of a solution of catalyst Ru-2 (30 μL of a 0.10 M solution in THF). The reaction mixture was heated to 40° C. and stirred for 4h. The solution was cooled to room temperature and then quenched with an excess of ethyl vinyl ether (0.5 mL, 5.2 mmol). The solvent was removed in vacuo and the crude mixture analyzed by LC/MS to measure the extent of conversion. (R_{f} of cross product=0.32 in 1:1 DCM:EtOAc+10% MeOH). HRMS (ESI) m/z calcd for C₁₄₈H₂₂₀N₂₂O₂₉S₂ [M+2H]²⁺: 1418.7929. found 1418.7948.

### Solid Phase Synthesis of Peptides

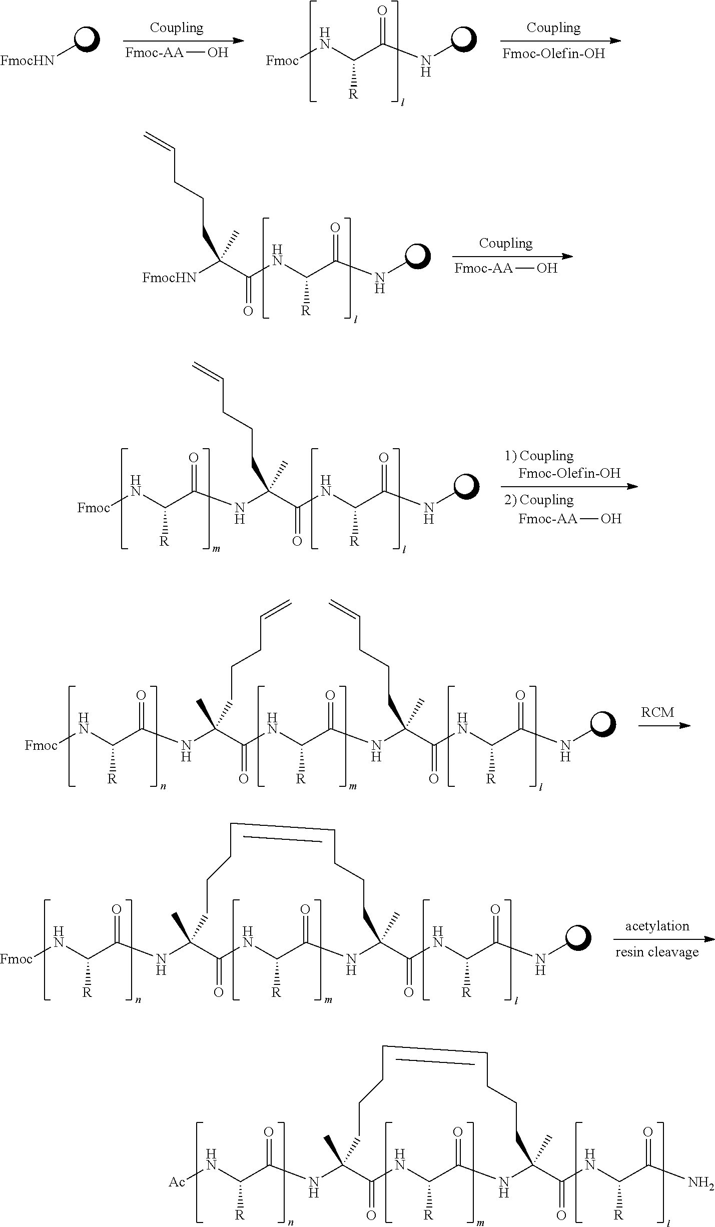

Peptides were produced on a Titan 357 (AAPPTec, Louisville, Ky.) automated peptide synthesizer using Rink Amide MBHA resin (NovaBioChem, 0.4 mmol/g resin), Wang resin (NovaBioChem, 0.5 mmol/g resin), or TentaGel MB RAM resin (RappPolymere, 0.4 mmol/g resin) at 40 μmol scale. The resin was swelled with N-Methyl 2-pyrrolidinone (NMP, 10 mL) for 30 min before use. To load the first amino acid onto the resin, the resin-bound Fmoc-protecting group was removed by treatment with 25% (vol/vol) piperidine in NMP (2×10 min). Standard amino acids were coupled for 1 h using HATU as the activating agent (4 eq. based on loading capacity), Fmoc-protected amino acid (5 eq.), and N,N-diisopropylethylamine (DIEA, 10 eq.) in NMP. After each coupling or deprotection reaction, the resin was washed successively with DCM (1×1 min), NMP (1×1 min), DCM (1×1 min) and NMP (1×1 min). For the coupling of olefin amino acids, a reaction time of 2 h was used with Fmoc-(S)-2-(4-pentenyl)alanine (3 eq.) or Fmoc-(R)-2-(7-octenyl)alanine (3 eq.), HATU (3 eq.) and DIEA (6 eq.) in NMP. After the final amino acid coupling, the resin was washed with DCM (2×1 min) and dried in vacuo overnight.

### Sequence of Peptides Used in Z-Selective RCM

Peptide 16: Ac-Glu-Asp-Ile-Ile-Arg-Ile-S5*-Arg-Leu-Leu-S5*-Glu-Val-Gly-Asp

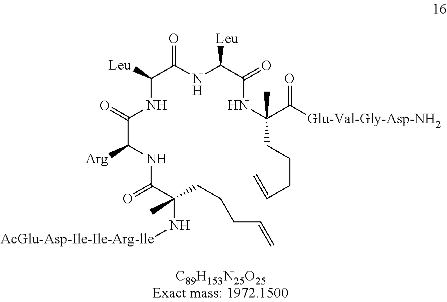

Peptide 18: Ac-Leu-Ser-Gln-Glu-Tyr-Phe-R8*-Asn-Leu-Trp-Lys-Leu-Leu-S5*-Gln-Asp

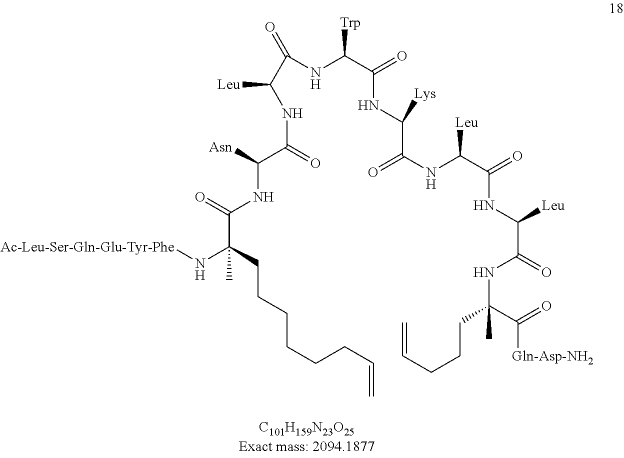

*S5 denotes position of (S)-2-(4-pentenyl)alanine
*R8 denotes position of (R)-2-(7-octenyl)alanine

### General Procedure for Z-Selective RCM on Resin-Bound Olefinic Peptides

The N-terminal modified peptide on resin (25 mg, 0.01 mmol) was dissolved in degassed dichloroethane (DCE, 2.0 mL). To this was added a stock solution of cyclometalated ruthenium catalyst Ru-2 in degassed DCE (20 μL of a 0.05 M solution in DCE). The reaction was stirred under a gentle stream of Ar(g) for 2 h, the catalyst was filtered off, and the resin washed first with DCE (5×2 min) and then with DMF (2×2 min). Exposure of the resin bound peptide to an additional round of catalyst stock solution (20 μL) for 2 h ensured nearly quantitative conversion. Upon completion of RCM, the resin bound peptide was washed with DCE (2×2 min), DMF (2×2 min), and DCM (2×2 min) and dried under vacuum.

For N-terminal acetylation of the peptide, the resin was swelled with NMP (1 mL) for 20 min and then washed with NMP (2×1 min). The resin was treated with 25% (vol/vol) piperidine in NMP (2 mL), gently agitated for 20 min, and then drained. The resin was washed with DCM (5×2 min) and allowed to dry under a gentle stream of argon to afford the amine-terminated peptide. To this was added NMP (1 mL), the resin was agitated for 10 min, and the solvent was drained. Acetic anhydride (30 μL, 0.3 mmol, 30 eq.) in NMP (1.0 mL) was added, followed by N,N-diisopropylethylamine (DIEA, 104 μL, 60 eq.) and the resin was agitated for 45 min at room temperature. The resin was washed with DCM (1×1 min), NMP (1×1 min), and DCM (1×1 min) and dried in vacuo overnight.

Cleavage of the peptide from the resin and global deprotection were achieved by reacting the resin with 95% TFA, 2.5% triisopropylsilane, 2.5% H₂O (vol/vol/vol) for 2 h. The TFA and other volatiles were removed by evaporation under a stream of argon. The peptides were precipitated with cold diethyl ether (4 mL), vortexed, and collected by centrifugation. The pellet was dried under a gentle stream of argon and subsequently dissolved in a mixture of 50% acetonitrile, 50% H₂O (vol/vol) and the resin was removed by filtration. The cleaved peptides were purified by reverse-phase HPLC using a Zorbax C₈ or C₁₈ column (Agilent, 5 μm, 9.4×250 mm) and characterized by LC/MS TOF using a Zorbax C₈ column (Agilent, 3.5 μm, 2.1×150 mm) or matrix-assisted laser desorption ionization time-of-flight (MALDI-TOF).

Representative Z-selective RCM across one turn of a helix.

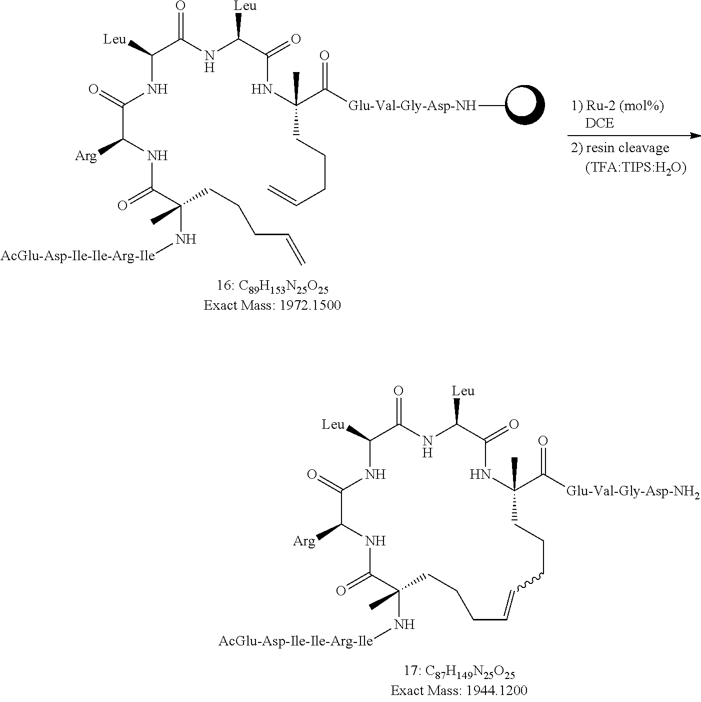

Representative Z-selective RCM across two helical turns.

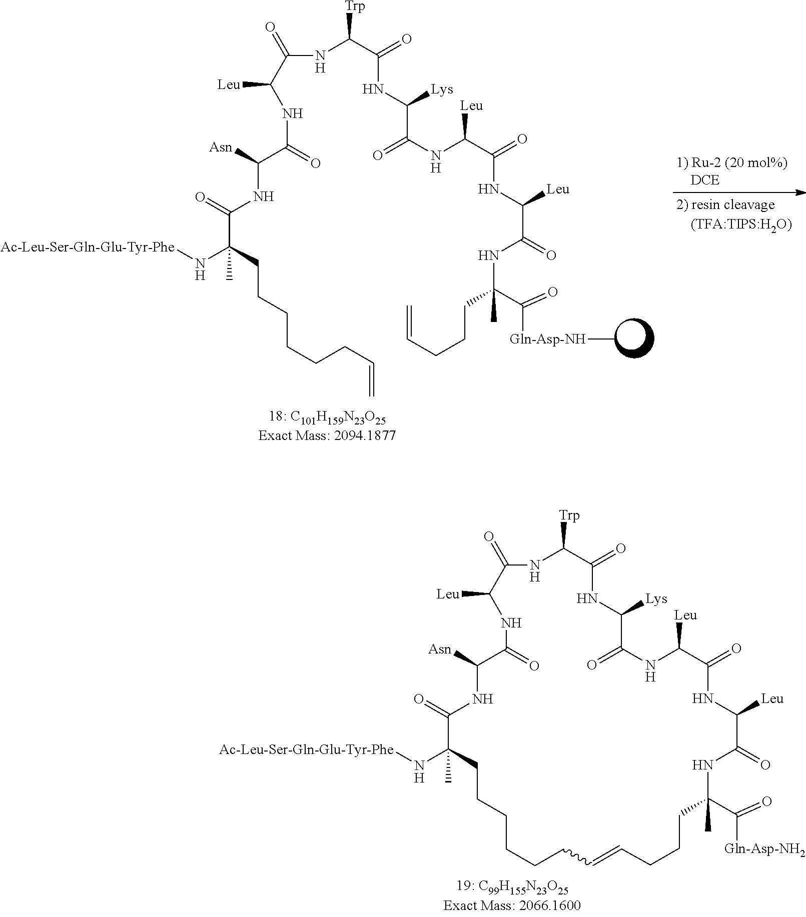

Monitoring the conversion of RCM on resin-bound olefinic peptides.

To measure the percentage conversion of RCM on peptides 16 and 18, aliquots of the resin suspension (25 μL) were taken from the reaction mixture at the indicated time points, quenched with ethyl vinyl ether (50 μL), filtered, and washed with DCE (300 μL). The resin was dried under a stream of argon and suspended in 100 μL of the cleavage cocktail TFA/TIS/H₂O (95:2.5:2.5) and allowed to stir at room temperature for 1 h. The TFA and other volatiles were removed by evaporation and the crude residue dissolved in diethyl ether (200 μL), vortexed, and centrifuged. The ether was carefully decanted and the pellet was dried under a stream of argon. The pellet was dissolved in 100 μL of 50% (vol/vol) aqueous acetonitrile and filtered to afford the crude peptide. For LC/MS TOF analysis, 5 μL of dissolved peptide was injected onto an analytical column (Eclipse Plus C₈ column (1.8 μm, 2.1×50 mm)) operating in positive electrospray ionization (ESI) mode.

General procedure for RCM on Aib-containing peptides bearing i, i+3 crosslinks.

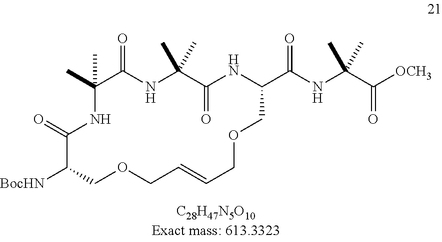

Boc-Ser(Allyl)-Aib-Aib-Ser(Allyl)-Aib-OMe 20 (for full characterization of this compound and its ring-closed form, see: Boal, A. K. et al. J. Am. Chem. Soc. 2007, 129, 6986-6987) (20.0 mg, 0.033 mmol) was dissolved in dichloroethane (6.5 mL) in a nitrogen-flushed flask. Second-generation Grubbs catalyst 22 (2.77 mg, 0.0033 mmol), Grubbs-Hoveyda catalyst 23 (2.04 mg, 0.0033 mmol), or cyclometalated ruthenium catalyst Ru-1 (6.11 mg, 0.099 mmol) was added in a single portion and then heated at 40° C. for 4 h. At this point, a 60 μL aliquot was removed and quenched by the addition of H₂O (3 mL) and 30% hydrogen peroxide (3 mL) and the biphasic mixture was vigorously stirred for 8 h. The organic layer was passed through a plug of Na₂SO₄ and an aliquot was removed for LCMS analysis. ¹HNMR (400 MHz, CD₂Cl₂): δ 7.48 (1H, d, J=7.4 Hz), 7.47 (1H, s), 6.96 (1H, s), 6.78 (1H, s), 5.74 (2H, m), 5.22 (1H, d, J=7.8), 4.56 (1H, dt, J=2.3, 8.7 Hz), 4.24 (1H, m), 4.16 (2H, m), 3.90 (1H, dd, J=2.9, 9.3 Hz), 3.822 (2H, m), 3.76 (1H, t, J=9.0 Hz), 3.65 (3H, s), 3.48 (1H, dd, J=4.3, 8.7 Hz), 1.55 (3H, s), 1.50 (3H, s), 1.46 (3H, s), 1.44 (15H, s), 1.42 (3H, s). ¹³C NMR (100 MHz, CD₂Cl₂): δ 175.26, 174.51, 174.46, 171.98, 169.44, 156.31, 132.33, 126.45, 81.17, 70.97, 70.23, 69.29, 66.70, 57.71, 57.45, 56.29, 55.18, 54.73, 52.42, 28.33, 27.78, 26.91, 25.23, 25.08, 23.55, 23.40. HRMS (ESI) m/z calcd for C₂₈H₄₈N₅O₁₀ [M+H]⁺: 614.3521. found 614.3533.

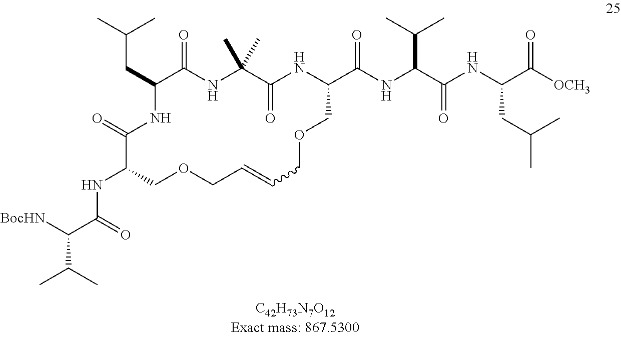

Following the procedure for RCM on peptide 21, Boc-Val-L-Ser(Al)-Leu-Aib-L-Ser(Al)-Val-Leu-OMe 24 (Boal, A. K. et al. J. Am. Chem. Soc. 2007, 129, 6986-6987) (9.1 mg, mmol) was dissolved in DCM (1.9 mL) under a stream of nitrogen. To this was added catalysts 22, 23, or Ru-1 (10 mol %) and the reaction heated at 40° C. for 4 h. The reaction was diluted with DCM (4 mL) and quenched by addition of water (2 mL) and 30% hydrogen peroxide (2 mL). The biphasic mixture was vigorously stirred for 4 h. An aliquot of the organic layer (60 μL) was removed for LCMS analysis. HRMS (FAB) m/z calcd for C₄₂H₄₈N₅O₁₀ [M+Na]⁺: 890.5209. found 890.5180.

What is claimed is:

1. A method for preparing at least one cross metathesis product, comprising: contacting a first olefin reactant with a second olefin reactant in the presence of a cyclometalated catalyst, under conditions effective to promote the formation of the at least one cross metathesis product; where the first olefin reactant and the second olefin reactant are each independently an optionally substituted amino acid comprising a terminal olefinic moiety or an optionally substituted peptide comprising a terminal olefinic moiety; and where the first olefin reactant and the second olefin reactant are the same or different.

2. The method of claim 1, wherein the at least one cross metathesis product is greater than about 80% Z.

3. The method of claim 1, wherein the optionally substituted amino acid comprising a terminal olefinic moiety or the optionally substituted peptide comprising a terminal olefinic moiety is represented by the structure of Formula (1): 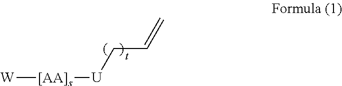
wherein,
AA is any amino acid residue;
U is CH₂, NH, O, or S;
W is hydrogen, a solid support, a functional group, or a protecting group;
t is 0-10; and
s is 1-10.

4. The method of claim 1, wherein the cyclometalated catalyst is represented by the structure of Formula (V), 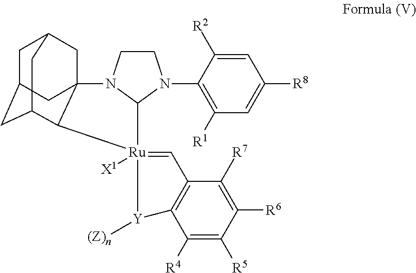
wherein,
R¹ is hydrogen, C₁-C₆ alkyl, substituted C₁-C₆ alkyl, C₁-C₆ heteroalkyl, substituted C₁-C₆ heteroalkyl; C₅-C₂₄ aryl, substituted C₅-C₂₄ aryl; C₅-C₂₄ heteroaryl, substituted C₅-C₂₄ heteroaryl; C₁-C₆ alkoxy, C₆-C₂₄ aralkyl, substituted C₆-C₂₄ aralkyl; C₆-C₂₄ alkaryl, substituted C₆-C₂₄ alkaryl, or halide, where the substituents are selected from C₁-C₆ alkyl, C₁-C₆ alkoxy, and halide;
R² is hydrogen, C₁-C₆ alkyl, substituted C₁-C₆ alkyl, C₁-C₆ heteroalkyl, substituted C₁-C₆ heteroalkyl; C₅-C₂₄ aryl, substituted C₅-C₂₄ aryl; C₅-C₂₄ heteroaryl, substituted C₅-C₂₄ heteroaryl; C₁-C₆ alkoxy, C₆-C₂₄ aralkyl, substituted C₆-C₂₄ aralkyl; C₆-C₂₄ alkaryl, substituted C₆-C₂₄ alkaryl, or halide, where the substituents are selected from C₁-C₆ alkyl, C₁-C₆ alkoxy, and halide;
R⁸ is selected from hydrogen, C₁-C₁₀ alkyl, substituted C₁-C₁₀ alkyl, C₅-C₁₀ aryl, substituted C₅-C₁₀ aryl, C₅-C₁₀ heteroaryl, substituted C₅-C₁₀ heteroaryl, halide (—Cl, —F, —Br, —I), hydroxyl, C₁-C₆ alkoxy, C₅-C₁₀ aryloxy, nitro (—NO₂), ester (—COOR⁹), ketone (—COR⁹), aldehyde (—COH), acyl (—COR⁹), ester (—OCOR⁹), carboxylic acid (—COOH), sulfonamide (—NR⁹SO₂Ar), carbamate (—NCO₂R⁹), cyano (—CN), sulfoxide (—SOR⁹), sulfonyl (—SO₂R⁹), sulfonic acid (—SO₃H), fluoromethyl (—CFₘ), fluroaryl (e.g., —C₆F₅, p-CF₃C₆H₄), where R⁹ is hydrogen, methyl, C₂-C₆ alkyl, substituted C₂-C₆ alkyl, C₅-C₁₀ aryl, or substituted C₅-C₁₀ aryl, wherein m is 1, 2, or 3;
X¹ is a bidentate anionic ligand;
Y is a heteroatom selected from N, O, S, and P;
R⁴, R⁵, R⁶, and R⁷ are each, independently, selected from hydrogen, halogen, alkyl, alkenyl, alkynyl, aryl, heteroalkyl, heteroatom containing alkenyl, heteroalkenyl, heteroaryl, alkoxy, alkenyloxy, aryloxy, alkoxycarbonyl, carbonyl, alkylamino, alkylthio, aminosulfonyl, monoalkylaminosulfonyl, dialkylaminosulfonyl, alkylsulfonyl, nitrile, nitro, alkylsulfinyl, trihaloalkyl, perfluoroalkyl, carboxylic acid, ketone, aldehyde, nitrate, cyano, isocyanate, hydroxyl, ester, ether, amine, imine, amide, halogen-substituted amide, trifluoroamide, sulfide, disulfide, sulfonate, sulfonamide, carbamate, silane, siloxane, phosphine, phosphate, or borate, wherein any combination of R⁴, R⁵, R⁶, and R⁷ can be linked to form one or more cyclic groups;
n is 1 or 2, such that n is 1 when Y is the divalent heteroatoms O or S, and n is 2 when Y is the trivalent heteroatoms N or P; and
Z is a group selected from hydrogen, alkyl, aryl, functionalized alkyl, functionalized aryl where the functional group(s) may independently be one or more or the following: alkoxy, aryloxy, halogen, carboxylic acid, ketone, aldehyde, nitrate, cyano, isocyanate, hydroxyl, ester, ether, amine, imine, amide, trifluoroamide, sulfide, disulfide, carbamate, silane, siloxane, phosphine, phosphate, or borate; methyl, isopropyl, sec-butyl, t-butyl, neopentyl, benzyl, phenyl and trimethylsilyl; and
wherein any combination or combinations of X¹, Q*, Y, Z, R⁴, R⁵, R⁶, and R⁷ may be optionally linked to a support.

5. The method of claim 2, wherein the at least one cross metathesis product is represented by the structure of Formula (4): 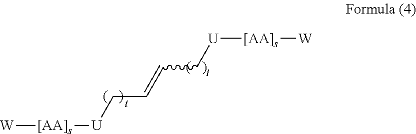
wherein,
AA is independently any amino acid residue;
W is independently hydrogen, a solid support, a functional group, or a protecting group;
U is independently CH₂, NH, O, or S;
t is independently 0-10; and
s is independently 1-10.

6. A method for preparing a ring-closing metathesis product, comprising: contacting a diolefin reactant with a cyclometalated catalyst under conditions effective to promote the formation of the ring-closing metathesis product; and where the diolefin reactant is an optionally substituted peptide comprising two terminal olefinic moieties.

7. The method of claim 6, wherein the ring-closing metathesis product is greater than about 80% Z.

8. The method of claim 7, wherein the ring-closing metathesis product is a macrocyclic peptide.

9. The method of claim 7, where the ring-closing metathesis product is a stapled peptide.

10. The method of claim 6, wherein the optionally substituted peptide comprising two terminal olefinic moieties is represented by the structure of Formula (8): 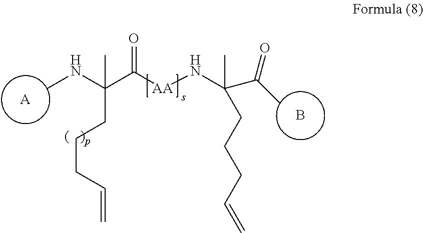
wherein,
AA is any amino acid residue;
A is hydrogen, a functional group, a protecting group, an optionally substituted amino acid residue, an optionally substituted peptide residue, a solid support, or any combination thereof;
B is hydrogen, a functional group, a protecting group, an optionally substituted amino acid residue, an optionally substituted peptide residue, a solid support, or any combination thereof;
p is 1-4; and
s is 1-10.

11. The method of claim 6, wherein the cyclometalated catalyst is represented by the structure of Formula (V), 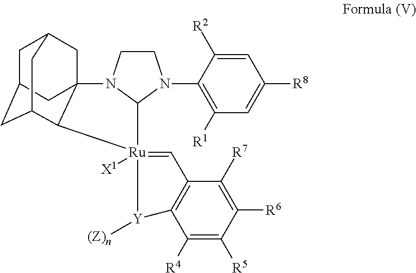
wherein,
R¹ is hydrogen, C₁-C₆ alkyl, substituted C₁-C₆ alkyl, C₁-C₆ heteroalkyl, substituted C₁-C₆ heteroalkyl; C₅-C₂₄ aryl, substituted C₅-C₂₄ aryl; C₅-C₂₄ heteroaryl, substituted C₅-C₂₄ heteroaryl; C₁-C₆ alkoxy, C₆-C₂₄ aralkyl, substituted C₆-C₂₄ aralkyl; C₆-C₂₄ alkaryl, substituted C₆-C₂₄ alkaryl, or halide, where the substituents are selected from C₁-C₆ alkyl, C₁-C₆ alkoxy, and halide;
R² is hydrogen, C₁-C₆ alkyl, substituted C₁-C₆ alkyl, C₁-C₆ heteroalkyl, substituted C₁-C₆ heteroalkyl; C₅-C₂₄ aryl, substituted C₅-C₂₄ aryl; C₅-C₂₄ heteroaryl, substituted C₅-C₂₄ heteroaryl; C₁-C₆ alkoxy, C₆-C₂₄ aralkyl, substituted C₆-C₂₄ aralkyl; C₆-C₂₄ alkaryl, substituted C₆-C₂₄ alkaryl, or halide, where the substituents are selected from C₁-C₆ alkyl, C₁-C₆ alkoxy, and halide;
R⁸ is selected from hydrogen, C₁-C₁₀ alkyl, substituted C₁-C₁₀ alkyl, C₅-C₁₀ aryl, substituted C₅-C₁₀ aryl, C₅-C₁₀ heteroaryl, substituted C₅-C₁₀ heteroaryl, halide (—Cl, —F, —Br, —I), hydroxyl, C₁-C₆ alkoxy, C₅-C₁₀ aryloxy, nitro (—NO₂), ester (—COOR⁹), ketone (—COR⁹), aldehyde (—COH), acyl (—COR⁹), ester (—OCOR⁹), carboxylic acid (—COOH), sulfonamide (—NR⁹SO₂Ar), carbamate (—NCO₂R⁹), cyano (—CN), sulfoxide (—SOR⁹), sulfonyl (—SO₂R⁹), sulfonic acid (—SO₃H), fluoromethyl (—CFₘ), fluroaryl (e.g., —C₆F₅, p-CF₃C₆H₄), where R⁹ is hydrogen, methyl, C₂-C₆ alkyl, substituted C₂-C₆ alkyl, C₅-C₁₀ aryl, or substituted C₅-C₁₀ aryl, wherein m is 1, 2, or 3;
X¹ is a bidentate anionic ligand;
Y is a heteroatom selected from N, O, S, and P;
R⁴, R⁵, R⁶, and R⁷ are each, independently, selected from hydrogen, halogen, alkyl, alkenyl, alkynyl, aryl, heteroalkyl, heteroatom containing alkenyl, heteroalkenyl, heteroaryl, alkoxy, alkenyloxy, aryloxy, alkoxycarbonyl, carbonyl, alkylamino, alkylthio, aminosulfonyl, monoalkylaminosulfonyl, dialkylaminosulfonyl, alkylsulfonyl, nitrile, nitro, alkylsulfinyl, trihaloalkyl, perfluoroalkyl, carboxylic acid, ketone, aldehyde, nitrate, cyano, isocyanate, hydroxyl, ester, ether, amine, imine, amide, halogen-substituted amide, trifluoroamide, sulfide, disulfide, sulfonate, sulfonamide, carbamate, silane, siloxane, phosphine, phosphate, or borate, wherein any combination of R⁴, R⁵, R⁶, and R⁷ can be linked to form one or more cyclic groups;
n is 1 or 2, such that n is 1 when Y is the divalent heteroatoms O or S, and n is 2 when Y is the trivalent heteroatoms N or P; and
Z is a group selected from hydrogen, alkyl, aryl, functionalized alkyl, functionalized aryl where the functional group(s) may independently be one or more or the following: alkoxy, aryloxy, halogen, carboxylic acid, ketone, aldehyde, nitrate, cyano, isocyanate, hydroxyl, ester, ether, amine, imine, amide, trifluoroamide, sulfide, disulfide, carbamate, silane, siloxane, phosphine, phosphate, or borate; methyl, isopropyl, sec-butyl, t-butyl, neopentyl, benzyl, phenyl and trimethylsilyl; and
wherein any combination or combinations of X¹, Q*, Y, Z, R⁴, R⁵, R⁶, and R⁷ may be optionally linked to a support.

12. The method of claim 7, wherein the ring-closing metathesis product is represented by the structure of Formula (9): 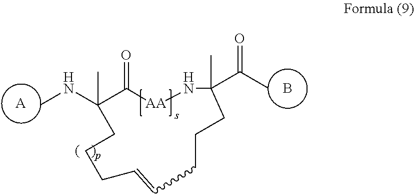
wherein,
AA is any amino acid residue;
A is hydrogen, a functional group, a protecting group, an optionally substituted amino acid residue, an optionally substituted peptide residue, a solid support, or any combination thereof;
B is hydrogen, a functional group, a protecting group, an optionally substituted amino acid residue, an optionally substituted peptide residue, a solid support, or any combination thereof;
p is 1-4; and
s is 1-10.
